(19) United States
(12) Patent Application Publication
(10) Pub. No.: US 2025/0279156 A1
(43) Pub. Date: Sep. 4, 2025

(54) METHODS FOR OPTIMISING PROTEIN PRODUCTION

(71) Applicant: United Kingdom Research and Innovation, Swindon, Wiltshire (GB)

(72) Inventors: Sebastian B. OEHM, Cambridgeshire (GB); Daniel L. DUNKELMANN, Cambridgeshire (GB); ADAM T. BEATTIE, Tokyo (JP); Jason W. CHIN, Cambridgeshire (GB)

(21) Appl. No.: 18/569,455

(22) PCT Filed: Jul. 14, 2022
(86) PCT No.: PCT/EP2022/069744

§ 371 Date: Dec. 12, 2023

(30) Foreign Application Priority Data
Jul. 14, 2021 (GB) 2110137.3

(51) Int. Cl. G16B 15/10; C12N 9/00

(52) U.S. Cl. CPC G16B 15/10; C12N 9/93

(57) ABSTRACT

The present invention relates to novel methods of optimising protein production. These methods include: methods of optimising orthogonal mRNAs, methods of designing and producing optimal operons comprising exogenous tRNAs, and methods of designing and producing optimal operons comprising exogenous genes, such as those encoding orthogonal aminoacyl-tRNA synthetases (O-aaRSs). The invention also relates to the products of said methods. Also provided as a part of the invention are host cells comprising the products of these innovations, methods of using said cells, and the products thereof. The host cells of the invention may be used for improved production of proteins and polypeptides comprising genetically incorporated non-canonical amino acids.

### FIELD OF THE INVENTION

The present invention relates to novel methods of optimising protein production. These methods include: methods of optimising orthogonal mRNAs, methods of designing and producing optimal operons comprising exogenous tRNAs, and methods of designing and producing optimal operons comprising exogenous genes, such as those encoding orthogonal aminoacyl-tRNA synthetases (O-aaRSs). The invention also relates to the products of said methods. Also provided as a part of the invention are host cells comprising the products of these innovations, methods of using said cells, and the products thereof. The host cells of the invention may be used for improved production of proteins and polypeptides comprising genetically incorporated non-canonical amino acids.

### BACKGROUND OF THE INVENTION

The ability to genetically encode the incorporation of multiple distinct non-canonical amino acids (ncAAs) into proteins will provide new opportunities for the engineering and directed evolution of protein function, will enable new strategies for biological discovery and understanding biological processes, and will provide a foundation for the encoded cellular synthesis of non-canonical biopolymers^{1, 2}. Encoding multiple distinct ncAAs into proteins synthesized in cells requires orthogonal codons, beyond those used to encode natural protein synthesis in the same cell; these include quadruplet codons³⁻⁵, codons arising from sense codon compression^{6, 7}, and codons incorporating non-canonical bases⁸⁻¹¹. Orthogonal codons must be assigned to ncAAs using engineered mutually orthogonal aminoacyl-tRNA synthetase (aaRS)/tRNA pairs. These pairs should be orthogonal in their aminoacylation specificity with respect to the synthetases and tRNAs used by the host organism for natural translation, and with respect to other orthogonal aaRSs and tRNAs used to direct ncAAs in the same cell; moreover, they should specifically recognize distinct ncAA monomers and decode distinct orthogonal codons^{3, 12-18}.

Orthogonal ribosomes (O-ribosomes) are non-natural ribosomes that are directed towards an orthogonal mRNA (O-mRNA), which is not a substrate for wild-type (wt) ribosomes in Escherichia coli (E. coli). These ribosomes operate in parallel with natural ribosomes but contain alterations in their ribosomal RNA that direct them to an O-ribosome binding site (O-RBS) within the 5′ untranslated region (5′ UTR) of the orthogonal message¹⁹. Since O-ribosomes are not responsible for synthesizing the proteome, they can be engineered to perform new functions not accessed by natural ribosomes, including new decoding and new intrinsic polymerization functions^{3, 20, 21}. O-riboQ1 (an evolved O-ribosome) efficiently decodes amber codons and quadruplet codons on O-mRNAs, using cognate tRNAs, and thus provides orthogonal codons that are selectively decoded on the orthogonal message^{3, 20}.

Engineered mutually orthogonal aaRS/tRNA pairs—which recognize distinct ncAAs and decode distinct codons—have been used to incorporate two or three distinct ncAAs into proteins^{3, 4, 14, 15, 18, 22}. The homologous Methanosarcina mazei (Mm) or Methanosarcina barkeri (Mb) pyrrolysyl-tRNA widely used orthogonal aaRS/tRNA pairs for genetic code expansion^{2, 23}. The inventors recently investigated PylRS/tRNAPyl pairs from diverse organisms and discovered that natural PylRS and tRNAPyl sequences cluster into several subclasses with distinct specificities; this insight allowed the inventors to engineer doubly and triply orthogonal PylRS/tRNAPyl pairs that recognize distinct ncAAs and decode distinct codons^{14, 15}.

By combining O-riboQ1-mediated translation of O(trans)-strepGFP(40TAG, 136AGGA or 150AGTA)_{His6} (an O-mRNA for a _{Strep}GFP_{His6} open reading frame (ORF) translated from a previously described 5′ UTR containing an O-ribosome binding site (O(trans)), and containing two quadruplet codons (AGGA and AGTA) and an amber codon (TAG)) with engineered triply orthogonal PylRS/tRNA^{Pyl} pairs, the inventors demonstrated the incorporation of three ncAAs into recombinant StrepGFP(40BocK, 136NmH, 150CbzK)_{His6}¹⁵. However—as the inventors noted^{14, 15}—the yield of protein from this expression system was low and un-optimized. Additional experiments—with O(trans)-ₛₜᵣₑₚGFP_{His6} and a ₛₜᵣₑₚGFP_{His6} open reading frame with a 5′ UTR containing a wt RBS—demonstrated that the translation of O(trans)-ₛₜᵣₑₚGFP_{His6} by the O-ribosome leads to 31-fold less _{Strep}GFP_{His6} protein than is produced by wt ribosomes. Moreover, transferring the O(trans) 5′ UTR to other ORFs also leads to substantially decreased levels of protein synthesis (FIG. 1 and Supplementary FIG. 1). The O(trans) 5′ UTR sequence was derived from constructs for producing GST fusion proteins, where it directed O-ribosome dependent translation at comparable levels to O-ribosome independent translation from a 5′ UTR containing a wt RBS^{3, 20}. These observations demonstrated that—although the O(trans) sequence directs efficient orthogonal translation for some ORFs—it does not provide a general solution for the efficient translation of ORFs.

As such, general solutions for the creation of O-mRNAs that maximize protein yields in orthogonal translation are required.

### SUMMARY OF THE INVENTION

The inventors provide herein highly effective methods of optimising protein production.

In an aspect of the invention, there is provided a method of designing a messenger RNA (mRNA) which is an orthogonal messenger RNA (O-mRNA) suitable for translation by an orthogonal ribosome (O-ribosome), wherein the mRNA comprises a 5′ untranslated region (5′ UTR) and an open reading frame (ORF), the method comprising:
(a) predicting the free energy difference between the free-folded state of the mRNA and the O-ribosome-bound initiation-competent state of the mRNA (ΔGtot(O-ribo));
(b) introducing a modification into the 5′ UTR;
(c) predicting the new ΔGtot(O-ribo) (ΔGtotnew(O-ribo)) after modification;
(d) accepting the modification if said ΔGtotnew(O-ribo) is more negative than the preceding ΔGtot(O-ribo), and
accepting or rejecting the modification according to a probability distribution if said ΔGtotnew(O-ribo) is more positive than the preceding ΔGtot(O-ribo); and
(e) generating an O-mRNA sequence comprising the 5′ UTR which comprises the accepted modification(s).

ΔGₜₒₜ(O-ribo) may be the sum of the free energy required to unfold the mRNA (ΔG_{unfolding}) and the free energy released upon the mRNA binding to the O-ribosome to form an O-ribosome-bound initiation-competent state (ΔG_{o-ribo binding}).

The O-ribosome may comprise an orthogonal 16S rRNA and the mRNA may comprise a Shine Dalgarno sequence, and the ΔGₜₒₜ(O-ribo) may be predicted according to the following:

$$Δ {G}_{tot} ( O - ribo ) = ( Δ {G}_{mRNA - O - rRNA} + Δ {G}_{start} + Δ {G}_{spacing} - Δ {G}_{standby} ) + Δ {G}_{unfolding} ;$$

wherein
ΔG_{mRNA-O-rRNA} is the free energy of the predicted co-folded secondary structure of the last 9 nucleotides of the orthogonal 16S rRNA and the mRNA;
ΔGₛₜₐᵣₜ is the energy released from binding of an initiator tRNA to the start codon of the ORF;
ΔG_{spacing} is an energy penalty for non-optimal spacing length between the Shine Dalgarno sequence and the start codon;
ΔG_{standby} is the energy required to unfold secondary structures that sequester the four nucleotides upstream of the Shine Dalgarno sequence; and
ΔG_{unfolding} is the energy required to unfold secondary structures in the mRNA.

If the ΔGₜₒₜ^{new}(O-ribo) is more positive than the preceding ΔGₜₒₜ(O-ribo), the magnitude of the difference between said ΔGₜₒₜ^{new}(O-ribo) and said ΔGₜₒₜ(O-ribo) may determine the probability of acceptance, wherein a smaller magnitude is associated with a higher chance of acceptance compared to a larger magnitude.

The probability distribution according to which the modification is accepted or rejected may be:

$$exp ( \frac{\vert Δ {G}_{tot}^{new} ( O - ribo ) - Δ {G}_{tot} ( O - ribo ) \vert}{{T}_{SA}} )$$

wherein T_{SA} is the simulated annealing temperature.

The T_{SA} may be adjusted to maintain a 5-20% acceptance rate.

In an embodiment, the method is for designing an mRNA which is an O-mRNA suitable for translation by an O-ribosome in a cell also comprising a second ribosome (2^{nd}-ribosome), wherein
step (a) comprises predicting the free energy difference between the free-folded state of the mRNA and the 2^{nd}-ribosome-bound initiation-competent state of the mRNA (ΔGₜₒₜ(2^{nd}-ribo));
step (c) comprises predicting the new ΔGₜₒₜ(2^{nd}-ribo) (ΔGₜₒₜ^{new}(2^{nd}-ribo) after modification;
step (d) is: accepting the modification if said ΔGₜₒₜ^{new}(O-ribo) is more negative than the preceding ΔGₜₒₜ(O-ribo) and said ΔGₜₒₜ^{new}(2^{nd}-ribo) is more positive than the preceding ΔGₜₒₜ(2^{nd}-ribo), and
accepting or rejecting the modification according to a probability distribution if said ΔGₜₒₜ^{new}(O-ribo) is more positive than the preceding ΔGₜₒₜ(O-ribo) or if said ΔGₜₒₜ^{new}(2^{nd}-ribo) is more negative than the preceding ΔGₜₒₜ(2^{nd}-ribo).

In a particular embodiment, there is provided a method of designing an mRNA which is an O-mRNA suitable for translation by an O-ribosome in a cell also comprising a second ribosome (2^{nd}-ribosome), wherein the mRNA comprises a 5′ UTR and an ORF, wherein the method comprises:
(a) predicting the free energy difference between the free-folded state of the mRNA and the O-ribosome-bound initiation-competent state of the O-mRNA (ΔGₜₒₜ(O-ribo)) and predicting the free energy difference between the free-folded state of the mRNA and the 2^{nd}-ribosome-bound initiation-competent state of the mRNA (ΔGₜₒₜ(2^{nd}-ribo));
(b) introducing a modification into the 5′ UTR;
(c) predicting the new ΔGₜₒₜ(O-ribo) (ΔGₜₒₜ^{new}(O-ribo)) and the new ΔGₜₒₜ(2^{nd}-ribo) (ΔGₜₒₜ^{new}(2^{nd}-ribo) after modification;
(d) accepting the modification if said ΔGₜₒₜ^{new}(O-ribo) is more negative than the preceding ΔGₜₒₜ(O-ribo) and said ΔGₜₒₜ^{new}(2^{nd}-ribo) is more positive than the preceding ΔGₜₒₜ(2^{nd}-ribo), and
accepting or rejecting the modification according to a probability distribution if said ΔGₜₒₜ^{new}(O-ribo) is more positive than the preceding ΔGₜₒₜ(O-ribo) or if said ΔGₜₒₜ^{new}(2^{nd}-ribo) is more negative than the preceding ΔGₜₒₜ(2^{nd}-ribo); and
(e) generating an O-mRNA sequence comprising the 5′ UTR which comprises the accepted modification(s).

The ΔGₜₒₜ(2^{nd}-ribo) may be the sum of the free energy required to unfold the mRNA (ΔG_{unfolding}) and the free energy released upon the mRNA binding to the 2^{nd}-ribosome to form a 2^{nd}-ribosome-bound initiation-competent state (ΔG_{2nd ribo binding}).

The 2^{nd}-ribosome may comprise a 16S rRNA and the mRNA may comprise a Shine Dalgarno sequence, and the ΔGₜₒₜ(2^{nd}-ribo) may be predicted according to the following:

$$Δ {G}_{tot} ( {2}^{nd} - ribo ) = ( Δ {G}_{mRNA - 2 nd - rRNA} + Δ {G}_{start} + Δ {G}_{spacing} - Δ {G}_{standby} ) + Δ {G}_{unfolding} ;$$

wherein
ΔG_{mRNA-2nd-rRNA} is the free energy of the predicted co-folded secondary structure of the last 9 nucleotides of the 16S rRNA and the mRNA;
ΔGₛₜₐᵣₜ is the energy released from binding of an initiator tRNA to the start codon of the ORF;
ΔG_{spacing} is an energy penalty for non-optimal spacing length between the Shine Dalgarno sequence and the start codon;
ΔG_{standby} is the energy required to unfold secondary structures that sequester the four nucleotides upstream of the Shine Dalgarno sequence; and
ΔG_{unfolding} is the energy required to unfold secondary structures in the mRNA.

In an embodiment, when the ΔGₜₒₜ^{new}(O-ribo) is more positive than the preceding ΔGₜₒₜ(O-ribo) or the ΔGₜₒₜ^{new}(2^{nd}-ribo) is more negative than the preceding ΔGₜₒₜ(2^{nd}-ribo), the magnitude of the difference between said ΔGₜₒₜ^{new}(O-ribo) and said ΔGₜₒₜ(O-ribo) or between said ΔGₜₒₜ^{new}(2^{nd}-ribo) and said ΔGₜₒₜ(2^{nd}-ribo) determines the probability of acceptance, wherein a smaller magnitude is associated with a higher chance of acceptance compared to a larger magnitude.

In an embodiment:
step (a) comprises calculating ΔGₜₒₜ(opt) according to the formula: ΔGₜₒₜ(opt)=ΔGₜₒₜ(O-ribo)−X*ΔGₜₒₜ(2^{nd}-ribo);
step (c) comprises calculating ΔGₜₒₜ^{new}(opt) according to the formula: ΔGₜₒₜ^{new}(opt)=ΔGₜₒₜ^{new}(O-ribo)−X*ΔGₜₒₜ^{new}(2^{nd}-ribo); and
step (d) is: accepting the modification if said ΔGₜₒₜ^{new}(opt) is more negative than the preceding ΔGₜₒₜ(opt), and
accepting or rejecting the modification according to a probability distribution if said ΔGₜₒₜ^{new}(opt) is more positive than the preceding ΔGₜₒₜ(opt);
wherein X is from 0.1 to 2, or X is 0.5.

In an embodiment, when the ΔGₜₒₜ^{new}(opt) is more positive than the preceding ΔGₜₒₜ(opt), the magnitude of the difference between said ΔGₜₒₜ^{new}(opt) and said ΔGₜₒₜ(opt) determines the probability of acceptance, wherein a smaller magnitude is associated with a higher chance of acceptance compared to a larger magnitude.

The probability distribution according to which the modification is accepted or rejected may be:

$$exp ( \frac{Δ {G}_{tot}^{new} ( opt ) - Δ {G}_{tot} ( opt )}{{T}_{SA}} )$$

wherein T_{SA} is the simulated annealing temperature.

The T_{SA} may be adjusted to maintain a 5-20% acceptance rate.

The modification may be or may comprise a single nucleotide change, insertion, or deletion.

In an embodiment, step (b) comprises introducing a modification into the 5′ UTR, or the exchange of any one of codons 2 to 20, 2 to 15, 2 to 12, 2 to 10, or 2 to 5 within the ORF with a synonymous codon; and step (e) comprises generating an O-mRNA sequence comprising the 5′ UTR and the ORF which comprise the accepted modification(s).

In an embodiment, step (b) comprises introducing a modification comprising a single nucleotide change, insertion, or deletion into the 5′ UTR, or the exchange of any one of codons 2 to 12 within the ORF with a synonymous codon.

In embodiments, steps (b) to (d) are iterated at least 200, 300, 400, 500, 1000, 5000, or 10000 times; or steps (b) to (d) are iterated until at least 10, 50, 100, 250, or 500 consecutive iterations consecutive iterations do not lead to a more negative ΔGₜₒₜ^{new}(O-ribo); or steps (b) to (d) are iterated until at least 10, 50, 100, 250, or 500 consecutive iterations consecutive iterations do not lead to a more negative ΔGₜₒₜ^{new}(O-ribo) or a more positive ΔGₜₒₜ^{new}(2^{nd}-ribo); or steps (b) to (d) are iterated until at least 10, 50, 100, 250, or 500 consecutive iterations consecutive iterations do not lead to a more negative ΔGₜₒₜ^{new}(opt).

In an embodiment, the 5′ UTR of step (a) is 35 nucleotides in length; or wherein the modification is at any of 35 nucleotides of the 5′ UTR that are closest to the start codon. The 5′ UTR of step (a) may be according to a randomly generated sequence of nucleic acids. The 5′ UTR of step (a) may comprise a wild type Shine Dalgarno sequence.

The O-ribosome may comprise an orthogonal anti-Shine Dalgarno sequence and the 5′ UTR of step (a) may comprise an orthogonal Shine Dalgarno sequence (0-SD) that is predicted to be perfectly complementary to the orthogonal anti-Shine Dalgarno sequence.

In some embodiments, step (b) does not comprise introducing a modification into the five-nucleotide core of the O-SD.

The Shine Dalgarno sequence may be five nucleotides from the start codon of the ORF.

In an embodiment, the 2^{nd} ribosome is a wild type ribosome or the 2^{nd} ribosome is an O-ribosome which differs from the first O-ribosome.

The method of designing an O-mRNA may be implemented on a computer.

In an aspect of the invention, there is provided a method for producing a nucleic acid sequence encoding an exogenous protein for translation by an O-ribosome, wherein the sequence of an O-mRNA is designed according to any method of designing an O-mRNA disclosed herein, and then a nucleic acid molecule is produced encoding said sequence.

In an aspect of the invention, there is provided a system for designing an orthogonal messenger RNA (O-mRNA) for translation by an orthogonal ribosome (O-ribosome), the system comprising:
a processor; and
one or more computer-readable storage media having stored thereon instructions for execution on said processor to perform any method of designing an O-mRNA disclosed herein.

In an aspect of the invention, there is provided a computer program product comprising a non-transitory machine readable medium storing program code that, when executed by one or more processors of a computer system, causes the computer system to implement any method of designing an O-mRNA disclosed herein.

In another aspect of the invention, there is provided a method of designing an operon encoding at least two exogenous tRNAs for expression in a host cell comprising an endogenous genome encoding endogenous tRNAs, the method comprising:
(i) generating permutations of arrangements of the at least two exogenous tRNAs;
(ii) identifying, within the endogenous genome, adjacent pairs of endogenous tRNAs with the highest level of sequence identity to each adjacent pair of exogenous tRNAs within each permutation of the at least two exogenous tRNAs;
(iii) identifying the intergenic region in the endogenous genome between each of the identified adjacent pairs of endogenous tRNAs;
(iv) generating a plurality of sequences encoding each permutation of the at least two exogenous tRNAs and comprising the identified intergenic region(s) positioned between each associated adjacent pair of the exogenous tRNAs; and
(v) selecting a sequence from said plurality of sequences for inclusion in the operon encoding the at least two exogenous tRNAs.

The selection of step (v) may be made from ranked list of the plurality of sequences, wherein the ranked list is created by ranking each of the plurality of sequences based on the sum of the sequence identity between the at least two exogenous tRNAs and the corresponding endogenous tRNAs used to define the intergenic regions.

The sequence identity of step (ii) may be calculated by comparing the acceptor stem sequences of the endogenous tRNAs to the acceptor stem sequences of the exogenous tRNAs. The first seven and last eight nucleotides, not including the CCA end, of the tRNAs may be compared.

The minimum intergenic region to be considered may be 5, 10, 15, 20, or 25 base pairs and the maximum may be 50, 75, 100, 125, or 150 base pairs. In an embodiment, the minimum intergenic region to be considered is 10 base pairs and the maximum is 100 base pairs.

The method may be for designing an operon encoding at least three, at least four, at least five, or at least six exogenous tRNAs.

Any of the methods of designing an operon encoding at least two exogenous tRNAs may be implemented on a computer.

In an aspect of the invention, there is provided a method for producing a nucleic acid sequence encoding an operon comprising at least two exogenous tRNAs, wherein the sequence of the nucleic acid is designed according to any of the methods of designing an operon encoding at least two exogenous tRNAs disclosed herein, and then a nucleic acid is produced encoding said sequence.

In an aspect of the invention, there is provided a system for designing an operon comprising at least two exogenous tRNAs, the system comprising:
a processor; and
one or more computer-readable storage media having stored thereon instructions for execution on said processor to perform any of the methods of designing an operon encoding at least two exogenous tRNAs disclosed herein.

In an aspect of the invention, there is provided a computer program product comprising a non-transitory machine readable medium storing program code that, when executed by one or more processors of a computer system, causes the computer system to implement any of the methods of designing an operon encoding at least two exogenous tRNAs disclosed herein.

In an aspect of the invention, there is provided a nucleic acid, wherein nucleic acid comprises an operon that is obtained or is obtainable by any of the methods of designing an operon encoding at least two exogenous tRNAs disclosed herein.

In an aspect of the invention, there is provided a host cell comprising an endogenous genome, wherein the host cell comprises a nucleic acid encoding an operon comprising at least two exogenous tRNAs, and wherein the nucleic acid sequence between each pair of exogenous tRNAs is an intergenic sequence derived from the endogenous genome.

The host cell may comprise an operon is obtained or is obtainable by any of the methods of designing an operon encoding at least two exogenous tRNAs disclosed herein.

The host cell may be a prokaryotic cell, such as a bacterial cell. The bacterial cell is may be E. coli and the endogenous genome may be an E. coli genome.

In another aspect of the invention, there is provided a method of designing an operon comprising at least two exogenous ORFs for expression in a host cell, wherein the method comprises:
(i) generating a plurality of 5′ UTR sequences for each of the at least two exogenous ORFs, wherein each 5′ UTR sequence is optimised for a negative predicted free energy difference between the free-folded state of an mRNA comprising said 5′ UTR sequence and the exogenous ORF and the ribosome-bound initiation-competent state of said mRNA (ΔGₜₒₜ(ribo));
(ii) predicting the ΔGₜₒₜ(ribo) for each of the 5′ UTR sequences when positioned 5′ to the exogenous ORF for which said 5′ UTR was optimised and positioned 3′ to each one of the remaining at least two exogenous ORFs; and
(iii) selecting an arrangement of the 5′ UTR sequences and the at least two exogenous ORFs.

Step (iii) may comprise selecting an arrangement of the 5′ UTR sequences and the at least two exogenous ORFs wherein:
the sum of the ΔGₜₒₜ(ribo) for all 5′ UTR/exogenous ORF pairs is the most negative; and/or
the mean of the ΔGₜₒₜ(ribo) for all 5′ UTR/exogenous ORF pairs is the most negative; and/or
each 5′ UTR/exogenous ORF pair has a ΔGₜₒₜ(ribo) which is more negative than a target ΔGₜₒₜ(ribo).

Step (i) may comprise generating two, three, four, five, or more 5′ UTR sequences for each of the at least two exogenous ORFs.

In an embodiment, at least one or all of the at least two exogenous ORFs is an aminoacyl-tRNA synthetase.

The method may be for designing an operon encoding at least three, at least four, at least five, or at least six exogenous ORFs.

ΔGₜₒₜ(ribo) may be the sum of the free energy required to unfold the mRNA (ΔG_{unfolding}) and the free energy released upon the mRNA binding to a ribosome to form a ribosome-bound initiation-competent state (ΔG_{ribo binding}).

ΔGₜₒₜ(ribo) may be predicted according to the following:

$$Δ {G}_{tot} ( ribo ) = ( Δ {G}_{mRNA - rRNA} + Δ {G}_{start} + Δ {G}_{spacing} - Δ {G}_{standby} ) + Δ {G}_{unfolding} ;$$

wherein
ΔG_{mRNA-rRNA} is the free energy of a predicted co-folded secondary structure of the last 9 nucleotides of a 16S rRNA and the mRNA;
ΔGₛₜₐᵣₜ is the energy released from binding of an initiator tRNA to the start codon of the sequence encoding the exogenous ORF;
ΔG_{spacing} is an energy penalty for non-optimal spacing length between the Shine Dalgarno sequence and the start codon of the sequence encoding the exogenous ORF;
ΔG_{standby} is the energy required to unfold secondary structures that sequester the four nucleotides upstream of the Shine Dalgarno sequence; and
ΔG_{unfolding} is the energy required to unfold secondary structures in the mRNA.

In an embodiment, wherein step (i) comprises:
(a) introducing a modification into the 5′ UTR;
(b) predicting the new ΔGₜₒₜ(ribo) (ΔGₜₒₜ^{new}(ribo)) after modification;
(c) accepting the modification if said ΔGₜₒₜ^{new}(ribo) is more negative than the preceding ΔGₜₒₜ(ribo), and
accepting or rejecting the modification according to a probability distribution if said ΔGₜₒₜ^{new}(ribo) is more positive than the preceding ΔGₜₒₜ(ribo); and
(d) generating a 5′ UTR sequence comprising the accepted modification(s).

In an embodiment, when the ΔGₜₒₜ^{new}(ribo) is more positive than the preceding ΔGₜₒₜ(ribo), the magnitude of the difference between said ΔGₜₒₜ^{new}(ribo) and said ΔGₜₒₜ(ribo) determines the probability of acceptance, wherein a smaller magnitude is associated with a higher chance of acceptance compared to a larger magnitude.

The probability distribution according to which the modification is accepted or rejected may be:

$$exp ( \frac{\vert Δ {G}_{tot}^{new} ( ribo ) - Δ {G}_{tot} ( ribo ) \vert}{{T}_{SA}} )$$

wherein T_{SA} is the simulated annealing temperature.

The T_{SA} may be adjusted to maintain a 5-20% acceptance rate.

The modification may be or may comprise a single nucleotide change, insertion, or deletion. In an embodiment, step (a) comprises introducing a modification into the 5′ UTR, or the exchange of any one of codons 2 to 20, 2 to 15, 2 to 12, 2 to 10, or 2 to 5 with a synonymous codon within the sequence encoding the exogenous ORF; and step (d) comprises generating a sequence comprising the 5′ UTR and the ORF which comprise the accepted modification(s). In a particular embodiment, step (a) comprises introducing a modification comprising a single nucleotide change, insertion, or deletion into the 5′ UTR, or the exchange of any one of codons 2 to 12 within the ORF with a synonymous codon.

Steps (a) to (c) may be iterated at least 200, 300, 400, 500, 1000, 5000, or 10000 times. Alternatively, steps (a) to (c) may be iterated until at least 10, 50, 100, 250, or 500 consecutive iterations consecutive iterations do not lead to a more negative ΔGₜₒₜ^{new}(ribo).

Any of the methods of designing an operon comprising at least two exogenous ORFs disclosed herein may implemented on a computer.

In an aspect of the invention, there is provided a method for producing a nucleic acid sequence encoding a polycistronic operon comprising at least two exogenous ORFs, wherein the sequence of the nucleic acid is designed according to any of the methods of designing an operon comprising at least two exogenous ORFs disclosed herein, and then a nucleic acid is produced according to said sequence.

In an aspect of the invention, there is provided a system for designing a polycistronic operon comprising at least two exogenous ORFs, the system comprising:
a processor; and
one or more computer-readable storage media having stored thereon instructions for execution on said processor to perform any of the methods of designing an operon comprising at least two exogenous ORFs disclosed herein.

In an aspect of the invention, there is provided a computer program product comprising a non-transitory machine readable medium storing program code that, when executed by one or more processors of a computer system, causes the computer system to implement any of the methods of designing an operon comprising at least two exogenous ORFs disclosed herein.

In an aspect of the invention, there is provided a nucleic acid, wherein nucleic acid comprises an operon that is obtained or is obtainable by any of the methods of designing an operon comprising at least two exogenous ORFs disclosed herein.

In an aspect of the invention, there is provided a host cell comprising a nucleic acid encoding an operon that is obtained or is obtainable by any of the methods of designing an operon comprising at least two exogenous ORFs disclosed herein.

The host cell may be a prokaryotic cell, such as a bacterial cell. The bacterial cell may be E. coli and the endogenous genome may be an E. coli genome.

In an aspect of the invention, there is provided a host cell comprising:
a nucleic acid sequence encoding an O-mRNA which encodes an exogenous protein, wherein the O-mRNA is obtained or is obtainable by any of the methods of designing an O-mRNA disclosed herein, and wherein the O-mRNA comprises at least two types of orthogonal codon;
a nucleic acid sequence comprising an O-tRNA operon encoding at least two orthogonal tRNAs, wherein the at least two orthogonal tRNAs are capable of decoding said at least two types of orthogonal codon, wherein the operon is obtained or is obtainable by any of the methods of designing an O-tRNA operon disclosed herein;
a nucleic acid sequence comprising an orthogonal aminoacyl-tRNA synthetase (O-aaRS) operon encoding at least two O-aaRSs, wherein the at least two O-aaRSs form O-aaRS-O-tRNA pairs with the at least two orthogonal tRNAs, wherein the operon is obtained or is obtainable by any of the methods of designing an operon encoding at least two exogenous genes disclosed herein; and
an orthogonal ribosome.

In an embodiment,
the O-mRNA comprises at least three types of orthogonal codon;
the O-tRNA operon encodes at least three orthogonal tRNAs which are capable of decoding said at least three orthogonal codons;
the O-aaRS operon encodes at least three O-aaRSs which form O-aaRS-O-tRNA pairs with the at least three orthogonal tRNAs.

In an embodiment,
the O-mRNA comprises at least four types of orthogonal codon;
the O-tRNA operon encodes at least four orthogonal tRNAs which are capable of decoding said at least four orthogonal codons;
the O-aaRS operon encodes at least four O-aaRSs which form O-aaRS-O-tRNA pairs with the at least four orthogonal tRNAs.

The host cell may be a prokaryotic cell, such as a bacterial cell. The bacterial cell may be E. coli and the endogenous genome may be an E. coli genome.

In an aspect of the invention, there is provided a method of producing a polypeptide, comprising:
providing a host cell comprising an O-ribosome, a O-tRNA operon, and an O-aaRS operon as disclosed herein;
incubating the host cell in the presence of a first non-canonical amino acid, wherein the first non-canonical amino acid is a substrate for the one of the O-aaRSs; and
incubating the host cell to allow incorporation of the first non-canonical amino acid into the polypeptide via the O-aaRS-O-tRNA pair.

In an embodiment, the method comprises:
incubating the host cell in the presence of a second non-canonical amino acid, wherein the second non-canonical amino acid is a substrate for the one of the O-aaRSs; and
incubating the host cell to allow incorporation of the second non-canonical amino acid into the polypeptide via the O-aaRS-O-tRNA pair.

In an embodiment, the method comprises:
incubating the host cell in the presence of a third non-canonical amino acid, wherein the third non-canonical amino acid is a substrate for the one of the O-aaRSs; and
incubating the host cell to allow incorporation of the third non-canonical amino acid into the polypeptide via the O-aaRS-O-tRNA pair.

In an embodiment, the method comprises:
incubating the host cell in the presence of a fourth non-canonical amino acid, wherein the fourth non-canonical amino acid is a substrate for the one of the O-aaRSs; and
incubating the host cell to allow incorporation of the fourth non-canonical amino acid into the polypeptide via the O-aaRS-O-tRNA pair.

In another aspect of the invention, there is provided a polypeptide obtained or obtainable by any method of producing a polypeptide disclosed herein.

### BRIEF DESCRIPTION OF THE DRAWINGS

FIG. 1. Automated discovery of O-mRNA sequences that are specifically and efficiently translated by O-ribosomes.

a, A thermodynamic model for the initiation of protein synthesis by wt and O-ribosomes on an mRNA. The free energy for the formation of the initiation complex (ΔGₜₒₜ) is the sum of the free energy required to unfold the mRNA (ΔG_{unfolding}) and the free energy released (ΔG_{ribo binding}) when the mRNA forms the initiation complex through binding to a ribosomal 30S subunit and tRNA^{fMet}_{CAU} (black trident and yellow star). The 30S subunit of an O-ribosome (light brown) contains an orthogonal anti-Shine Dalgarno (O-aSD) at the 3′ end of the O-16S rRNA, while the 30S subunit of the wt ribosome (dark brown) contains a wt anti-Shine Dalgarno (wt aSD) at the 3′ end of its 16S rRNA. The free energy released on forming the initiation complex from unfolded mRNA with a wt and orthogonal 30S are ΔG_{wt ribo binding} and ΔG_{O-ribo binding} respectively. Details on the calculations are provided in Methods. ORF open reading frame (orange), start codon (purple), SD/O-SD Shine-Dalgarno sequence or an orthogonal version, respectively (green), spacing between SD/O-SD and start codon (blue). The remainder of the 5′ UTR is shown in grey.

b, Algorithms developed to predict O-mRNA sequences that are efficiently and specifically translated by the O-ribosome. Algorithm vol 1 generates a random 35-nucleotide 5′ UTR containing a wt SD sequence and predicts its ΔGₜₒₜ(O-ribo). In an iterative process, a mutation is introduced into the 5′ UTR (a single nucleotide change, insertion, or deletion). The algorithm then predicts a new orthogonal ΔGₜₒₜ^{new}(O-ribo). If ΔGₜₒₜ^{new}(O-ribo) is more negative than ΔGₜₒₜ (O-ribo), the change is accepted; if the mutation leads to a more positive ΔGₜₒₜ^{new}(O-ribo), the change is rejected with some conditional probability (see Methods). The algorithm terminates after 10,000 iterations. Algorithm vol 2 generates a random 35-nucleotide 5′ UTR containing a O-SD sequence at an optimal 5-nucleotide spacing from the start codon and predicts its ΔGₜₒₜ(wt ribo) and ΔGₜₒₜ(O-ribo). In an iterative process, a mutation is introduced into the 5′ UTR (a single nucleotide change, insertion, or deletion). The algorithm then calculates new predicted values, ΔGₜₒₜ^{new}(wt ribo) and ΔGₜₒₜ^{new}(O-ribo). If ΔGₜₒₜ^{new}(wt ribo) is more positive than ΔGₜₒₜ(wt ribo) and ΔGₜₒₜ^{new}(O-ribo) is more negative than ΔGₜₒₜ(O-ribo), the change is accepted; otherwise, the mutation is rejected with some conditional probability (see Methods). If 500 consecutive iterations fail to yield improved ΔGₜₒₜ values (convergence criterium), then the algorithm outputs the sequence and its predicted ΔGₜₒₜ values. Algorithm vol 3 builds on vol 2, but has two notable differences: (1) Vol 3 also starts with an ORF in which codons 2 to 12 are randomly exchanged with synonymous codons, such that the encoded amino acid sequence is conserved. (2) In the iterative process, synonymous codon substitutions in the ORF are allowed mutation mechanisms in addition to single nucleotide changes, insertions or deletions in the 5′ UTR.

c, Algorithms discover O-mRNA sequences that are specifically and efficiently translated by O ribosomes. The y axis shows the production of ₛₜᵣₑₚGFP_{His6} from O-mRNAs by O-ribosomes; the data is shown as a percentage of ₛₜᵣₑₚGFP_{His6} produced by wt ribosomes from a wt message. The x axis shows the orthogonality of the O-mRNA; this is calculated as: ₛₜᵣₑₚGFP_{His6} produced from the O-mRNA in the presence of O-ribosomes divided by ₛₜᵣₑₚGFP_{His6} produced from the O-mRNA in the presence of wt ribosomes. Protein production levels are calculated from GFP absorption and fluorescence data; in our system the wt system generate 30.6±1.6 mg/mL of ₛₜᵣₑₚGFP_{His6}. Each dot represents one O-mRNA. Trans (black dot) is O(trans)-strepGFPHis6. The coloured dots represent sequences from the indicated volume of the algorithm. d, e, Same as in c but done for E2Crimson (d) and mCherry (e) respectively.

FIG. 2 Efficient production of proteins containing three distinct ncAAs is enabled by new O-mRNAs.

a, Structures of the amino acids used in this work. N⁶-(tert-butoxycarbonyl)-L-lysine (BocK) 1; N^{π}-methyl-L-histidine (NmH) 2; N⁶-((benzyloxy)carbonyl)-L-lysine (CbzK) 3; N⁶-((allyloxy)carbonyl)-L-lysine (AllocK) 4; (S)-2-amino-3-(4-iodophenyl)propanoic acid (PheI) 5.

b, Engineered triply orthogonal pyrrolysyl-tRNA synthetase tRNA pairs for the incorporation of three distinct ncAAs using two different orthogonal messages. One message contains the O1-ₛₜᵣₑₚGFP_{His6} 5′UTR, generated by vol 1 of our algorithm, and the other message used the O-(trans) 5′UTR.

c, Production of ₛₜᵣₑₚGFP(40BocK, 136NmH, 150CbzK)_{His6} from E. coli cells containing ₛₜᵣₑₚGFP(40TAG, 136AGGA and 150AGTA)_{His6} constructs with either the O(trans)- or O1-ₛₜᵣₑₚGFP-_{His6} 5′UTRs. Cells also contained O-riboQ1 and the aaRS3/tRNA3 operons (encoding MmPylRS/MspetRNA^{Pyl}_{CUA}, MlumPylRS(NMH)/MinttRNA^{Pyl-A17VC10}_{UCCU} and M1r26PylRS(CbzK)/MalvtRNA^{Pyl-8}_{UACU}). ncAAs BocK 1, NmH 2, CbzK 3 were added to the cell.

d, Results of positive electrospray TOF-MS of nickel-NTA purified ₛₜᵣₑₚGFP(40BocK, 136NmH, 150CbzK)_{His6} purified from cells described in (b). _{Strep}GFP(40BocK, 136NmH, 150Cbz)_{His6} mass predicted: 29314.5, mass found: 29312.0.

FIG. 3. Four orthogonal aaRS/tRNA pairs decoding four orthogonal quadruplet codons are expressed from aaRS operons and computationally generated tRNA operons and are mutually orthogonal in their aminoacylation specificity, recognize distinct ncAAs, and decode distinct orthogonal codons.

a-d, Fluorescence from cells containing O1-ₛₜᵣₑₚGFP(40XXXX)_{His6}, with XXXX being the codon at position 40 in sfGFP: TAGA, CTAG, AGGA or AGTA. E. coli also contained O-riboQ1 and the aaRS and tRNA operons (aaRS4_1-2/tRNA4(quad)); these operons expressed MmPylRS/MspetRNA^{Pyl-evol}_{UCUA}, MrumPylRS(NMH)/MinttRNA^{Pyl-A17VC10}_{UCCU}, AfTyrRS(PheI)/AftRNA^{Tyr-A01}_{CUAG} and Mg1PylRS(CbzK)/MalvtRNA^{Pyl-8}_{UACU}. The indicated ncAAs: N^{π}-methyl-L-histidine (NmH) 2, N⁶-((benzyloxy)carbonyl)-L-lysine (CbzK) 3, N⁶-((allyloxy)carbonyl)-L-lysine (AllocK) 4, (S)-2-amino-3-(4-iodophenyl)propanoic acid (PheI) 5 were added to cells or omitted (-). Each codon was only efficiently decoded in the presence of cognate ncAA of the aaRS/tRNA pair assigned to the respective quadruplet codon: (a) O1-ₛₜᵣₑₚGFP(TAGA)_{His6} decoded by MmPylRS/MspetRNA^{Pyl-evol}_{UCUA}, (b) O1-ₛₜᵣₑₚGFP(AGGA)_{His6} decoded by MrumPylRS(NMH)/MinttRNA^{Pyl-A17VC10}_{UCCU}, (c) O1-ₛₜᵣₑₚGFP(AGTA)_{His6} decoded by Mg1PylRS(CbzK)/MalvtRNA^{Pyl-8}_{UACU}, and (d) O1-ₛₜᵣₑₚGP-P(CTAG)_{His6} decoded by AfTyrRS(PheI)/AftRNA^{Tyr-A01}_{CUAG}.

e-h, Positive electrospray TOF-MS of nickel-NTA-purified ₛₜᵣₑₚGFP_{His6}, expressed from O1-ₛₜᵣₑₚGFP(40XXX)_{His6}, with XXXX being either TAGA (e), AGGA (f), AGTA (g) or CTAG (h), in the presence of NmH 2, CbzK 3, AllocK 4, PheI 5. Cells also contained O-riboQ1 and operon aaRS4_2-1/tRNA4(quad). strepGFP(40AllocK)_{His6} mass predicted 29113.2 mass found 29114.8. ₛₜᵣₑₚGFP(40NmH)_{His6} mass predicted 29052.1 mass found 29052.5. ₛₜᵣₑₚGFP(40CbzK)_{His6} mass predicted 29163.3 mass found 29164.2. ₛₜᵣₑₚGFP(40PheI)_{His6} mass predicted 29174.03 mass found 29174.2.

FIG. 4 Genetically encoding four distinct ncAAs into a protein using a 24 amino acid, 68 codon genetic code.

a, Schematic representation of four mutually orthogonal aaRS/tRNA pairs used for the incorporation of four distinct ncAAs in response to four orthogonal quadruplet codons. b, Efficient production of full length ₛₜᵣₑₚGFP(40PheI, 50AllocK, 136NmH, 150CbzK)_{His6} was dependent upon the addition of all four ncAAs (N^{π}-methyl-L-histidine (NmH) 2, N⁶-((benzyloxy)carbonyl)-L-lysine (CbzK) 3, N⁶-((allyloxy)carbonyl)-L-lysine (AllocK) 4, (S)-2-amino-3-(4-iodophenyl)propanoic acid (PheI) 5). Fluorescence from cells containing O1-ₛₜᵣₑₚGFP(40CTAG, 50TAGA, 136AGGA, 150AGTA)_{His6}, O-riboQ1, operon aaRS4/tRNA4(quad) (encoding MmPylRS/MspePyltRNA_{UCUA}, MrumPylRS(NMH)/MintPyltRNA(^{A}17,^{V}C10)_{UCCU}, AfTyrRS/AftRNA_{CUAG} and Mg1PylRS(CbzK)/MalvPyltRNA(8)_{UACU}) in presence or absence of a combination of NmH (2), CbzK (3), AllocK (4), PheI (5).

c, Positive electrospray TOF-MS of nickel-NTA purified ₛₜᵣₑₚGFP(40PheI, 50AllocK, 136NmH, 150CbzK)_{His6} from cells containing O1-ₛₜᵣₑₚGFP(40CTAG, 50TAGA, 136AGGA, 150AGTA)_{His6}, O-riboQ1 and aaRS4_1-2/tRNA4(quad) in presence of the indicated ncAAs. Mass predicted 29470.4 mass found 29468.2.

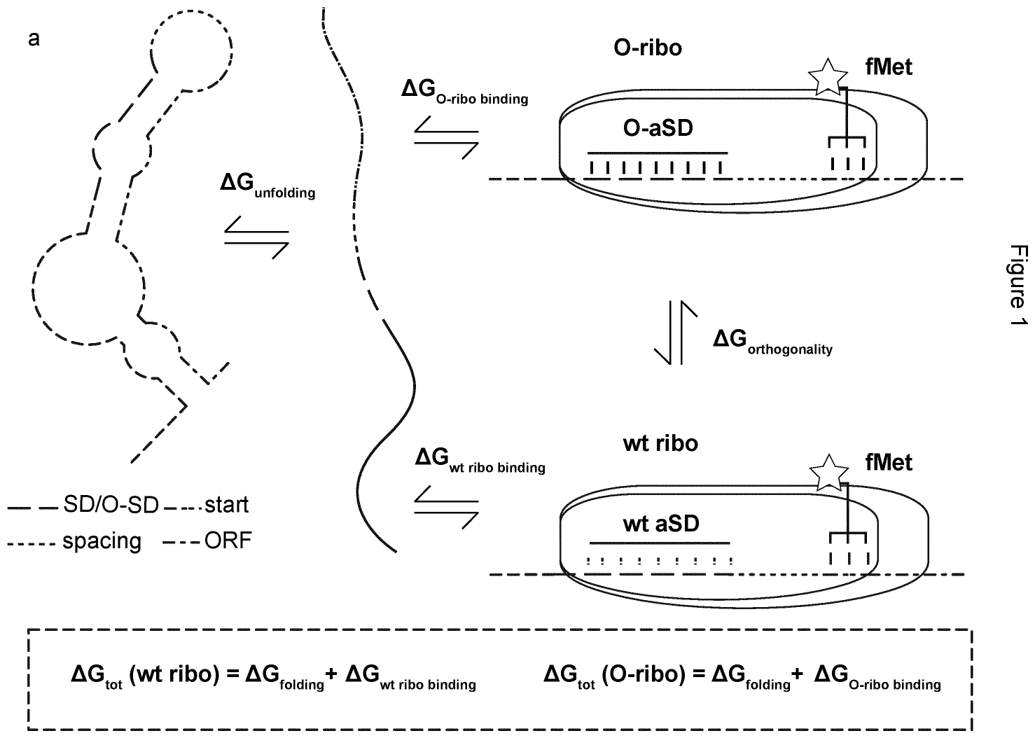

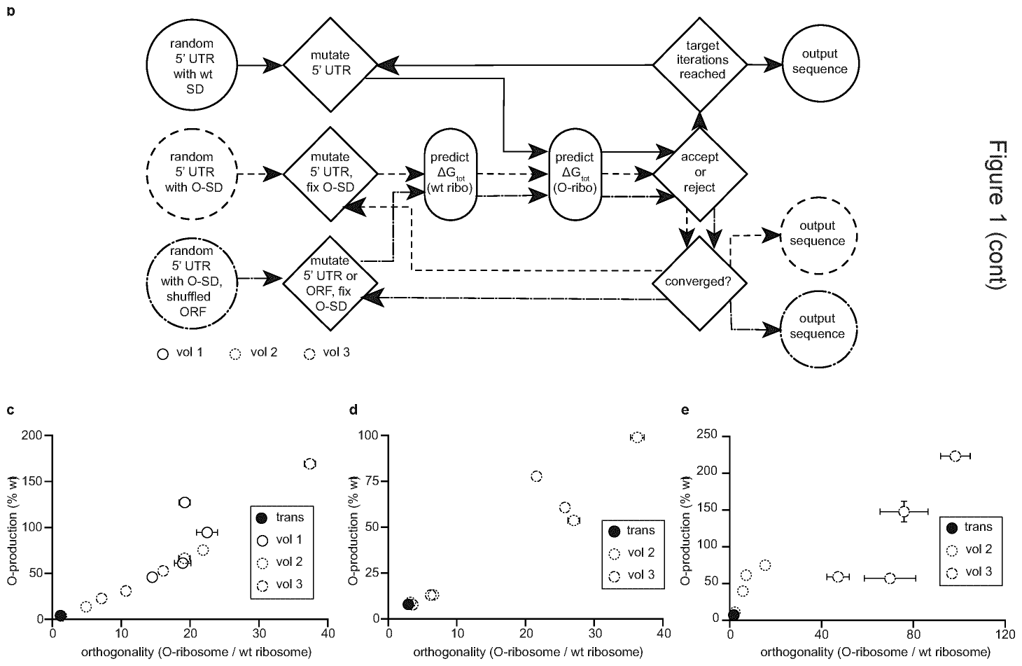

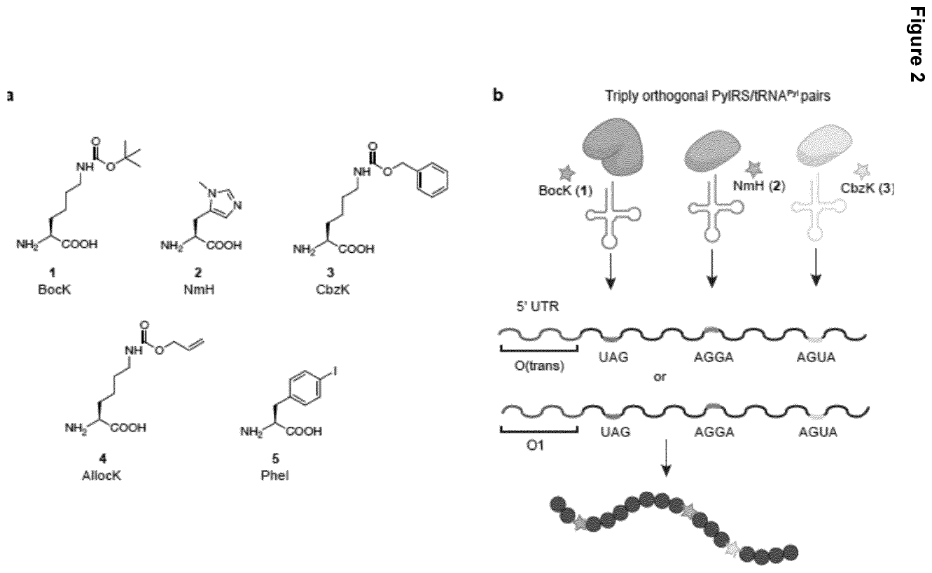

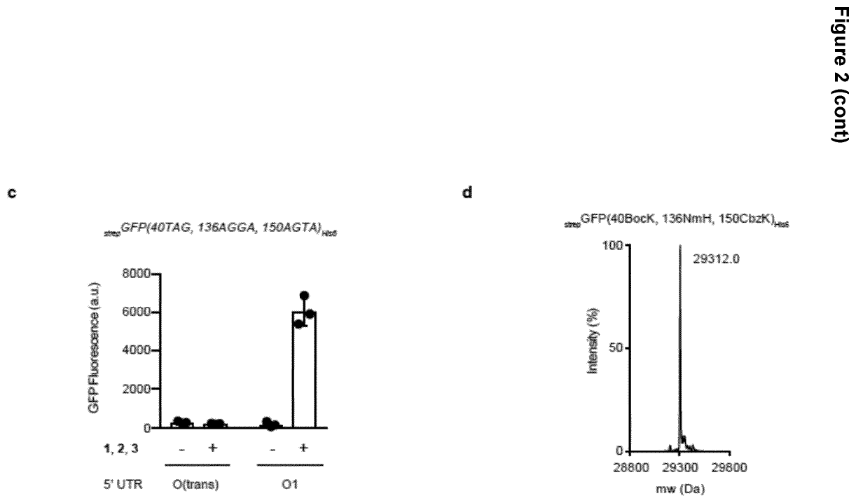

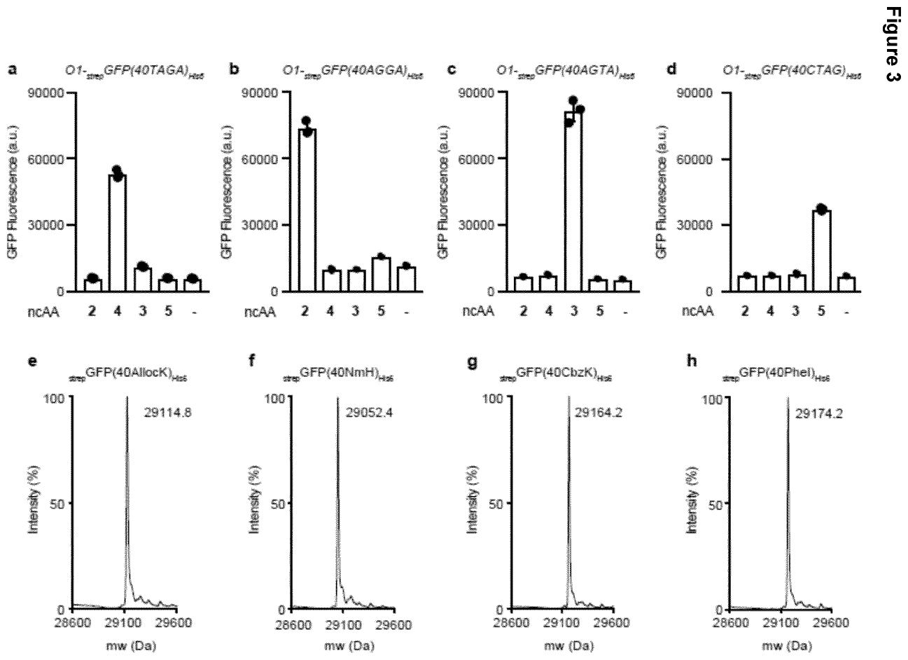

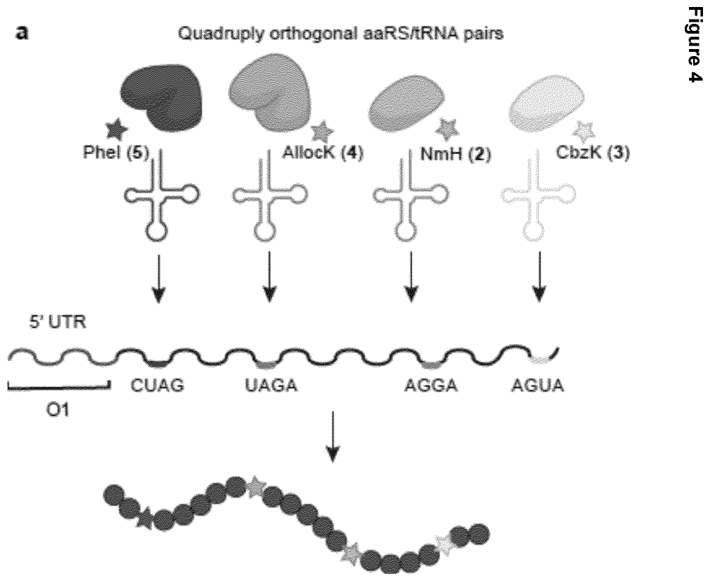

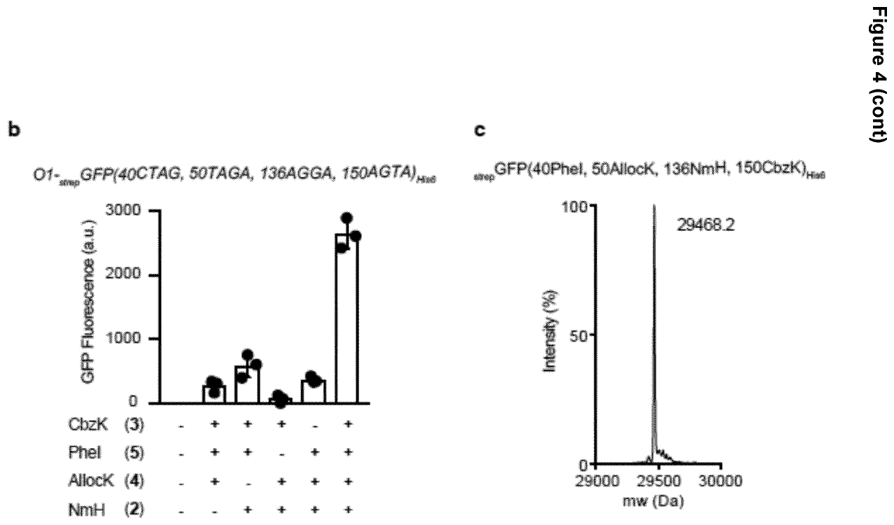

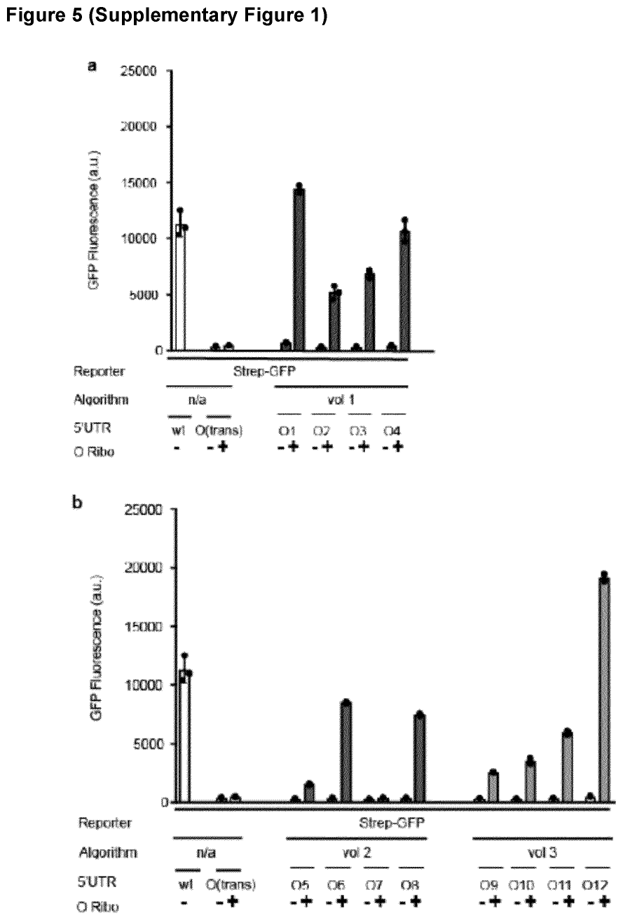

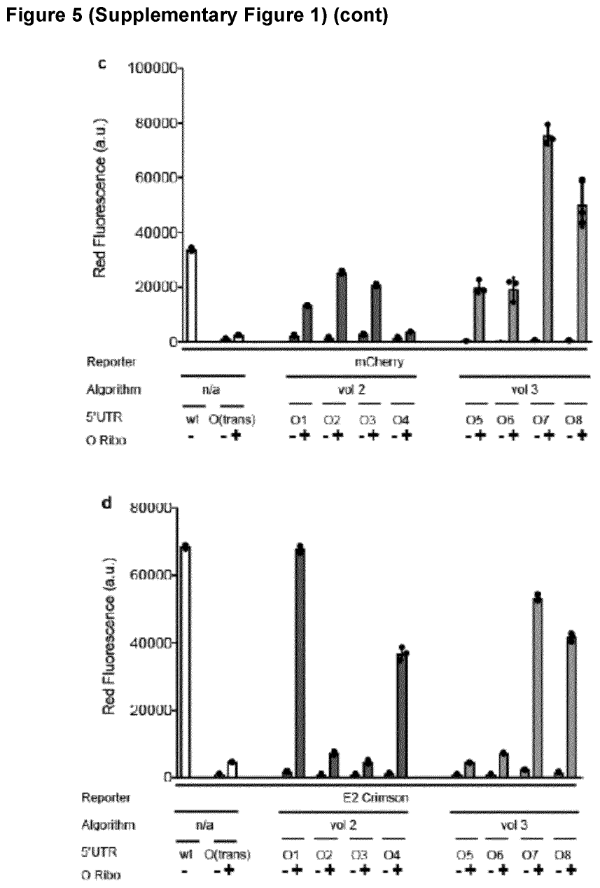

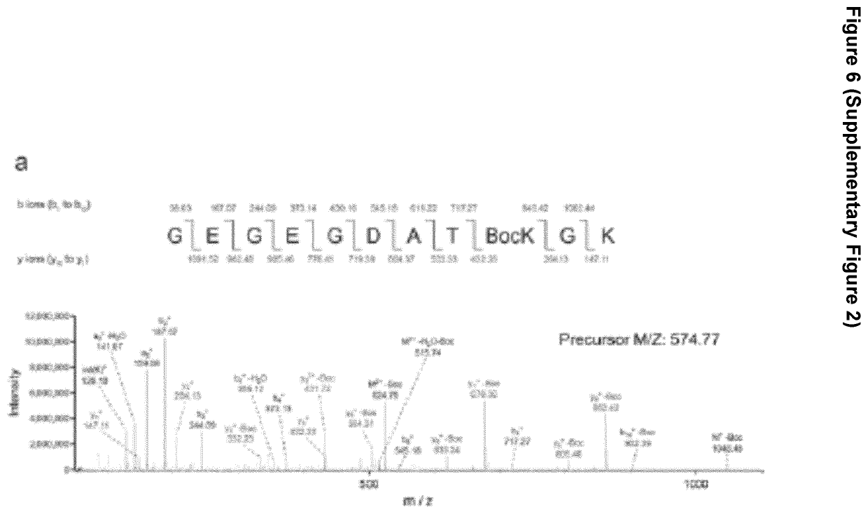

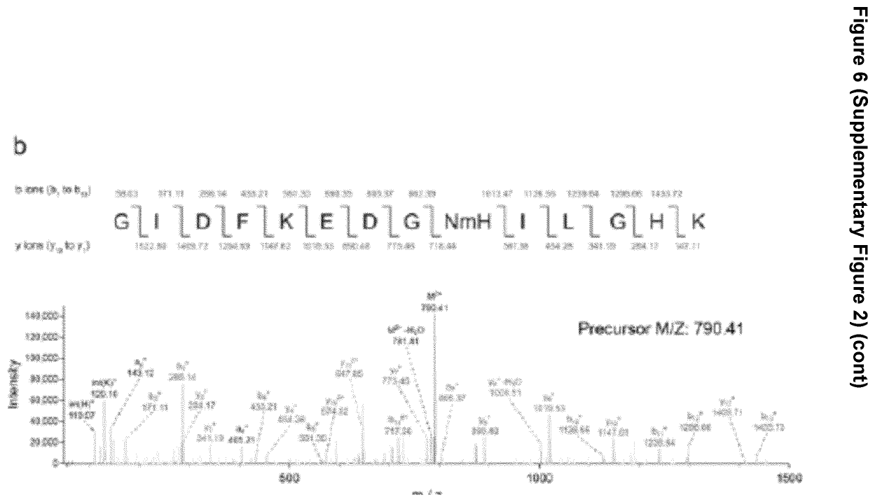

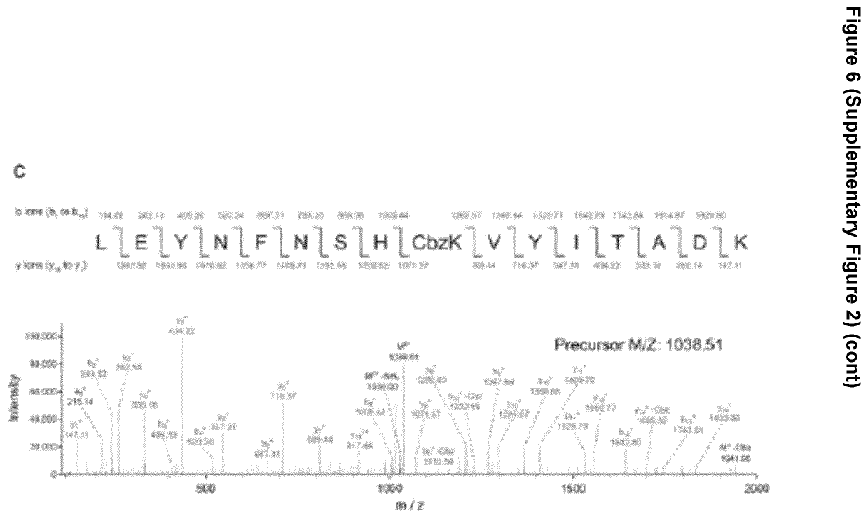

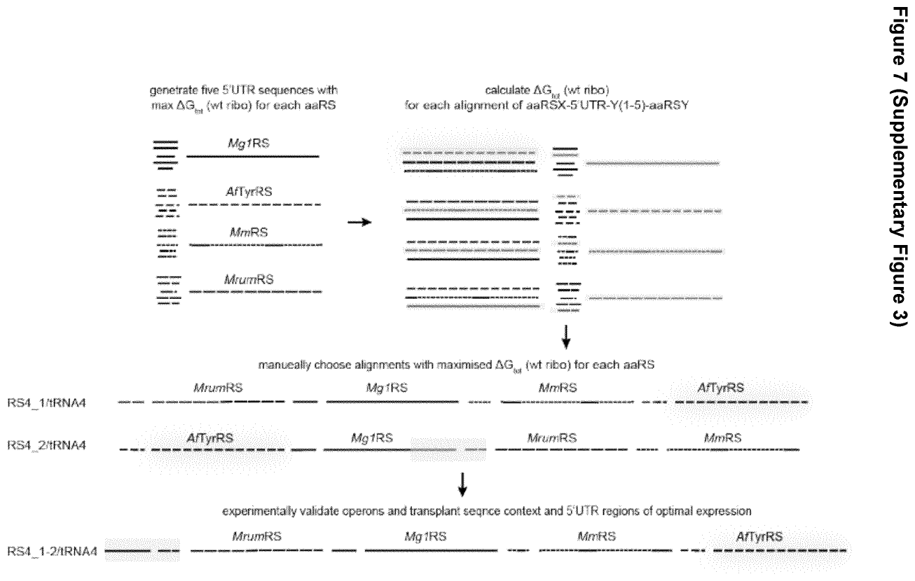

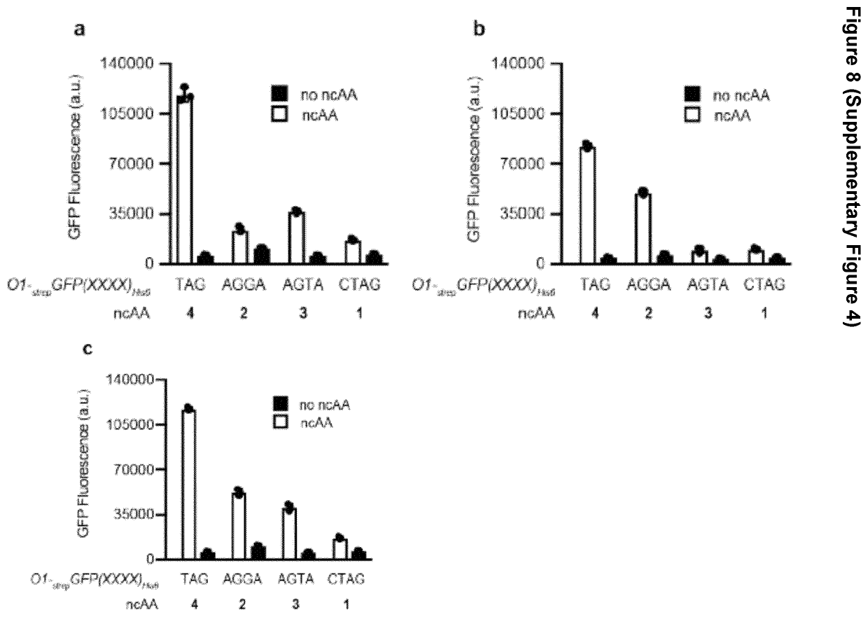

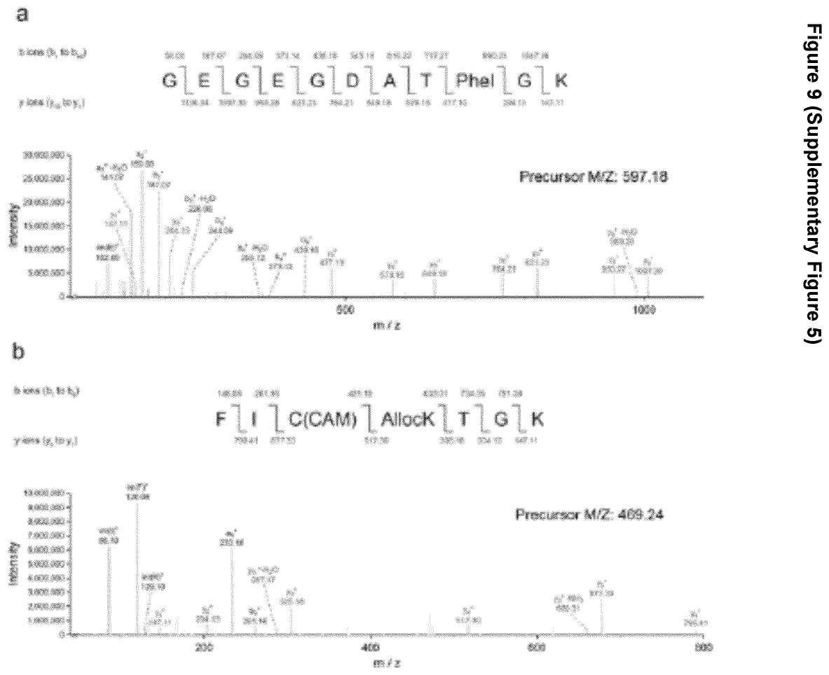

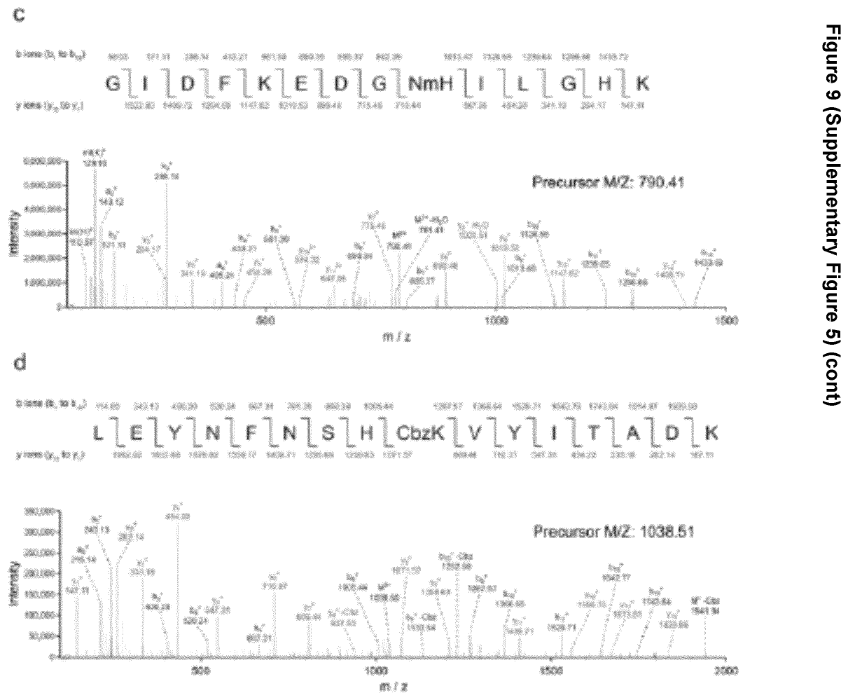

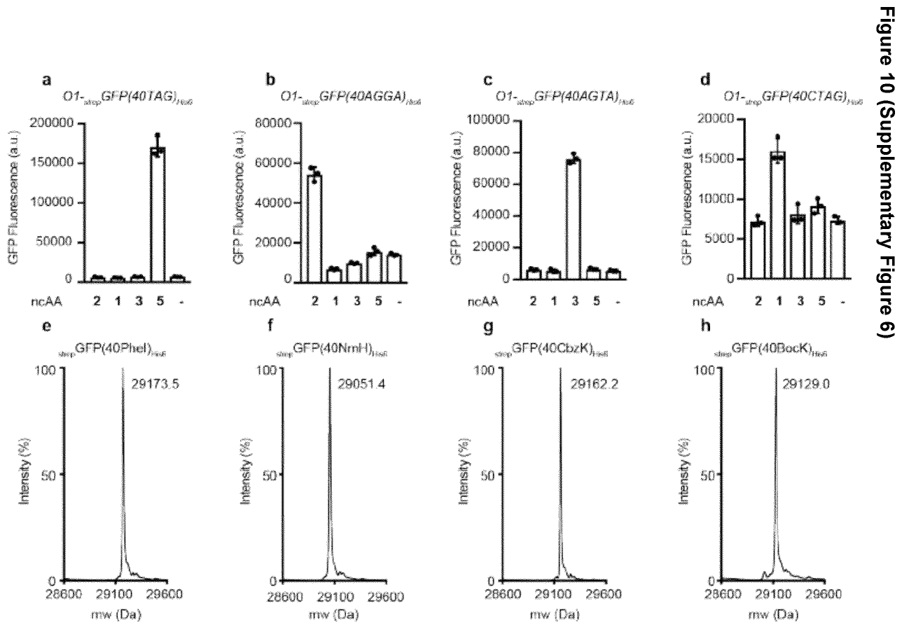

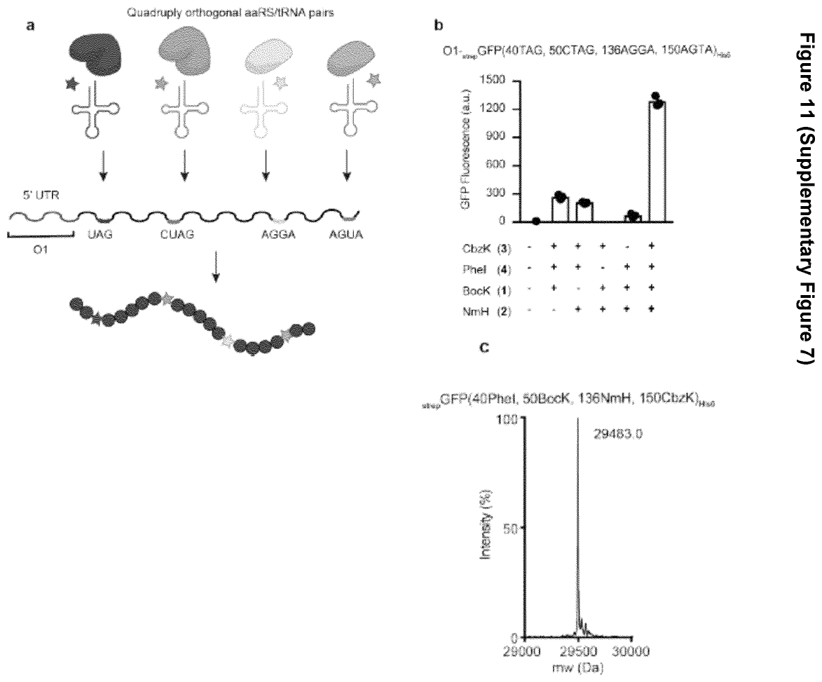

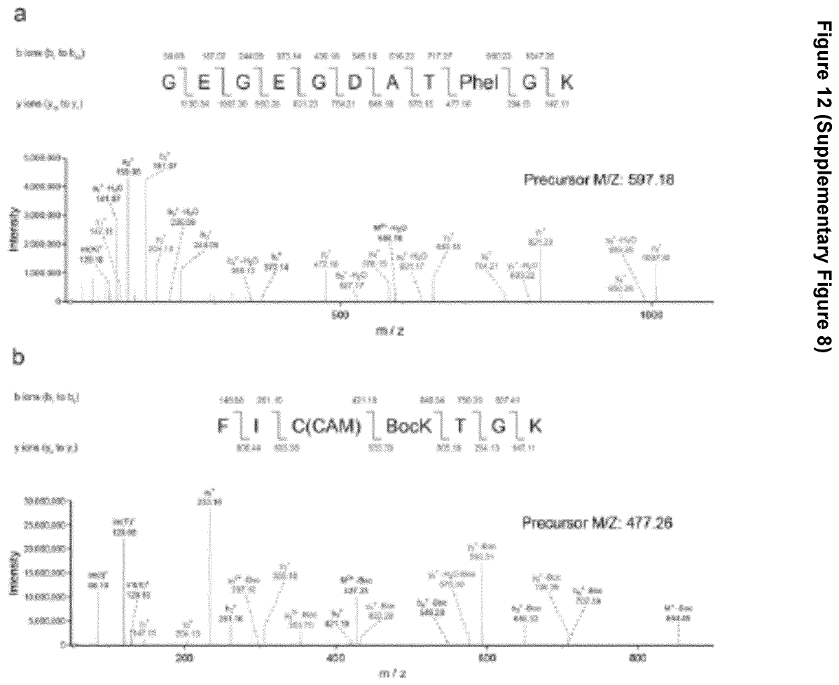

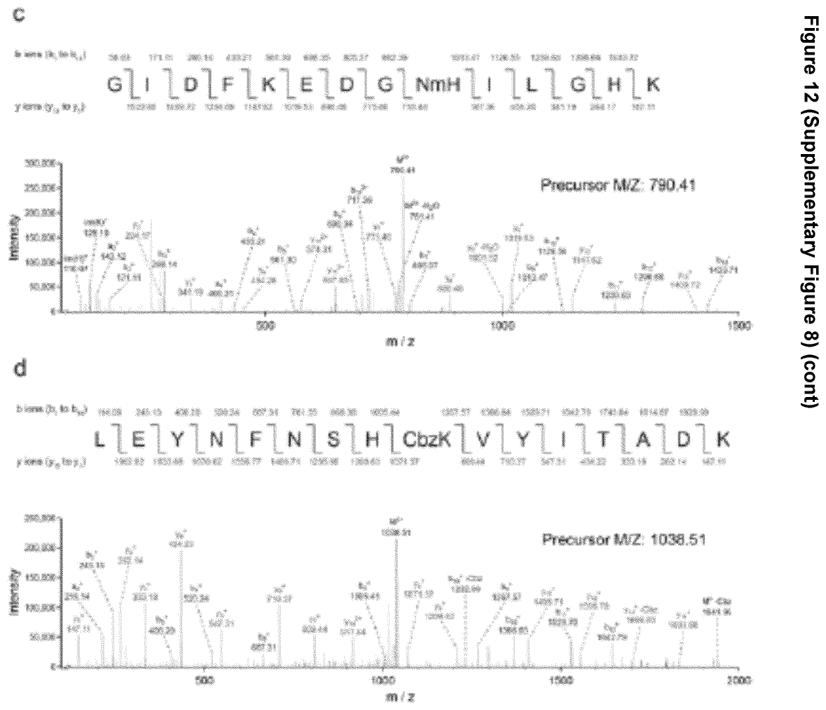

FIG. 5 (Supplementary FIG. 1)

Fluorescence measurements of reporter protein production by from O-mRNAs generated by the indicated algorithm. We cloned the sequences (O1-O12 ₛₜᵣₑₚGFP_{His6} for ₛₜᵣₑₚGFP_{His6} (a and b), O1-O8 E2Crimson for E2Crimson (c) as well as 01-08 mCherry for mCherry (d)) into a standardised p15A reporter construct, and produced proteins in the presence of a plasmid encoding either O-ribosome or an additional copy of the wt ribosome. Control experiments used a construct with a 5′ UTR and RBS commonly used in our lab (wt), and a construct with the O(trans) 5′ UTR previously used for highly efficient O-GST-CaM production^{3, 20}. Bars represent the mean of three biological replicates ±standard deviation. Dots represent individual experiments.

FIG. 6 (Supplementary FIG. 2)

MS/MS spectra of ncAA-containing peptides obtained following tryptic digest of ₛₜᵣₑₚGFP(40BocK, 136NmH, 150CbzK)_{His6}. The precursor ions confirm the incorporation of the ncAAs. Fragmentation of each peptide is predicted to yield a series of b ions (blue) and a series of y ions (red), as well as ions corresponding to the loss of the lysine protecting groups in the fragmentation process (a and c). Ion peaks were assigned manually; along with precursor ion masses, these confirmed the incorporation of each ncAA at its expected position. The mass spectrometry analysis was performed three times with similar results. a, MS/MS spectra confirming BocK 1 incorporation at position 40. b, MS/MS spectra confirming NmH 2 incorporation at position 136. c, MS/MS spectra confirming CbzK 3 incorporation at position 150.

FIG. 7 (Supplementary FIG. 3)

The assembly pipeline for the generation of polycistronic operons containing the genes for four mutually orthogonal aaRSs (AfTyrRS(PheI), MrumPylRS(NmH), Mg1PylRS(CbzK) and MmPylRS). For each synthetase, five 5′ UTR sequences were generated using the online RBS calculator^{27, 30, 31, 32, 33 (incorporated herein by reference)} optimised for max ΔGₜₒₜ(wt ribo). Then, ΔGₜₒₜ(wt ribo) for each alignment of the form aaRSX-5′_UTR(Y1-Y5)-aaRSY (where X and Y refer to any combination of two out of the four synthetases) was calculated using the online tool^{27, 30, 31, 32, 33}. Finally, all four synthetases were manually aligned in a way that guaranteed a high ΔGₜₒₜ(wt ribo) for each synthetase. Two independent solutions yielded similar results. After experimental validation, the favorable sequence context of one synthetase was copied into the other operon yielding the final construct (all 5′ UTR sequences and ΔGₜₒₜ(wt ribo) are given in Supplementary Table 3).

FIG. 8 (Supplementary FIG. 4)

Fluorescence from cells containing O1-ₛₜᵣₑₚGFP(XXXX)_{His6}, with XXXX being either TAG, CTAG, AGGA or AGTA. E. coli also contained O-riboQ1 and MmPylRS/MspePyltRNA_{CUAG}, MrumPylRS(NMH)/MintPyltRNA(^{A}17,^{V}C10)_{UCCU}, AfTyrRS/AfRNA_{CUA} and Mg1PylRS(CbzK)/MalvPyltRNA(8)_{UACU} and one of the ncAAs: NmH 2, CbzK 3, BocK 1 or PheI 5. Synthetases were initially either arranged in operons RS4_1/tRNA4 or RS4_2/tRNA4 (see Supplementary FIG. 3 and Supplementary Table 3). RS4_1/tRNA4 (a) yielded better results for the suppression of TAG, CTAG and AGTA; however, AGGA was only suppressed with half of the efficiency as in RS4_2/tRNA4 (b). Therefore 150 nt region upstream of MrumPylRS was copied into RS4_1/tRNA4 yielding operon 1 RS4_1-2/tRNA4 (c) leading to a 2.6 higher activity of MrumPylRS.

FIG. 9 (Supplementary FIG. 5)

MS/MS spectra of ncAA-containing peptides obtained following tryptic digest of ₛₜᵣₑₚGFP(40PheI, 50AllocK, 136NmH, 150CbZK)_{His6}. The precursor ions confirm the incorporation of the ncAAs. Fragmentation of each peptide is predicted to yield a series of b ions (blue) and a series of y ions (red), as well as ions corresponding to the loss of the lysine protecting groups in the fragmentation process (d). Ion peaks were assigned manually; along with precursor ion masses, these confirmed the incorporation of each ncAA at its expected position. The mass spectrometry analysis was performed three times with similar results. a, MS/MS spectra confirming PheI 5 incorporation at position 40. b, MS/MS spectra confirming AllocK 4 incorporation at position 50. c, MS/MS spectra confirming NmH 2 incorporation at position 136. d, MS/MS spectra confirming CbzK 3 incorporation at position 150.

FIG. 10 (Supplementary FIG. 6)

Four orthogonal aaRS/tRNA pairs decoding one amber codon and three orthogonal quadruplet codons are expressed from aaRS operons and computationally generated tRNA operons and are mutually orthogonal in their aminoacylation specificity, recognize distinct ncAAs, and decode distinct orthogonal codons. a-d, Fluorescence from cells containing O1-ₛₜᵣₑₚGFP(40XXXX)_{His6}, with XXXX being the codon at position 40 in sfGFP: TAG, CTAG, AGGA or AGTA. E. coli also contained ribo-Q1 and the aaRS and tRNA operons (aaRS4_1-2/tRNA4); these operons expressed MmPylRS/MspetRNA^{Pyl-evol}_{CUAG}, MrumPylRS(NMH)/MinttRNA^{Pyl-A17VC10}_{UCCU}, AfTyrRS(PheI)/AftRNA^{Tyr-A01}_{CUA} and Mg1PylRS(CbzK)/MalvtRNA^{Pyl-8}_{UACU}. The indicated ncAAs: N-methyl-L-histidine (NmH) 2, N⁶-((benzyloxy)carbonyl)-L-lysine (CbzK) 3, N⁶-(tertbutoxycarbonyl)-L-lysine (BocK) 1, (S)-2-amino-3-(4-iodophenyl)propanoic acid (PheI) 5 were added to cells or omitted (-). Each codon was only efficiently decoded in the presence of cognate ncAA of the aaRS/tRNA pair assigned to the respective quadruplet codon: (a) O1-ₛₜᵣₑₚGFP(TAG)_{His6} decoded by AfTyrRS(PheI)/AftRNA^{Try-A01}_{CUA}, (b) O1-ₛₜᵣₑₚGFP(AGGA)_{His6} decoded by MrumPylRS(NMH)/MinttRNA^{Pyl-A17VC10}_{UCCU}, (c) O1-ₛₜᵣₑₚGFP(AGTA)_{His6} decoded by Mg1PylRS(CbzK)/MalvtRNA^{Pyl-8}_{UACU}, and (d) O1-ₛₜᵣₑₚGFP(CTAG)_{His6} decoded by MmPylRS/MspetRNA^{Pyl-evol}_{CUAG}.

e-h, Positive electrospray TOF-MS of nickel-NTA-purified ₛₜᵣₑₚGFP_{His6}, expressed from O1-ₛₜᵣₑₚGFP(40XXX)_{His6}, with XXXX being either TAG (e), AGGA (f), AGTA (g) or CTAG (h), in the presence of NmH 2, CbzK 3, BocK 1, PheI 5. Cells also contained O-riboQ1 and operon aaRS4_2-1/tRNA4. ₛₜᵣₑₚGFP(40PheI)_{His6} mass predicted 29174.03 mass found 29174.2. ₛₜᵣₑₚGFP(40BocK)_{His6} mass predicted 29129.4 mass found 29129.0. ₛₜᵣₑₚGFP(40NmH)_{His6} mass predicted 29052.1 mass found 29052.5. ₛₜᵣₑₚGFP(40CbzK)_{His6} mass predicted 29163.3 mass found 29164.2. ₛₜᵣₑₚGFP(40BocK)_{His6} mass predicted 29129.4 mass found 29129.0.

FIG. 11 (Supplementary FIG. 7)

Genetically encoding four distinct ncAAs into a protein in response to an amber codon and three distinct quadruplet codons.

a, Schematic representation of four mutually orthogonal aaRS/tRNA pairs used for the incorporation of four distinct ncAAs in response to an amber codon and three distinct quadruplet codons.

b, Efficient production of full length ₛₜᵣₑₚGFP(40PheI, 50AllocK, 136NmH, 150CbzK)_{His6} was dependent upon the addition of all four ncAAs (BocK 1, NmH 2, CbzK 3 and PheI 5). Fluorescence from cells containing O1-ₛₜᵣₑₚGFP(40TAG, 5° C. TAG, 136AGGA, 150AGTA)_{His6}, O-riboQ1, operon aaRS4_1-2/tRNA4 (encoding MmPylRS/MspetRNA^{Pyl-evol}_{CUAG}, MrumPylRS(NMH)/MinttRNA^{Pyl-A17VC10}_{UCCU}, AfTyrRS(PheI)/AftRNA^{Tyr-A01}_{CUA} and Mg1PylRS(CbzK)/MalvtRNA^{Pyl-8}_{UACU}) in presence or absence of a combination of BocK 1, NmH 2, CbzK 3, PheI 5.

c, TOF-MS ES+ of purified ₛₜᵣₑₚGFP(40PheI, 50BocK, 136NmH, 150CbzK)_{His6} purified from cells containing O1-ₛₜᵣₑₚGFP(40TAG, 50CTAG, 136AGGA, 150AGTA)_{His6}, O-riboQ1 and operon RS4_1-2/tRNA4 in presence of 8 mM BocK 1, 4 mM NmH 2, 2 mM PheI 5 and 2 mM CbzK 3. Mass predicted 29482.0 mass found 29483.0.

FIG. 12 (Supplementary FIG. 8)

MS/MS spectra of ncAA-containing peptides obtained following tryptic digest of ₛₜᵣₑₚGFP(40PheI, 50BocK, 136NmH, 150CbzK)_{His6}. The precursor ions confirm the incorporation of the ncAAs. Fragmentation of each peptide is predicted to yield a series of b ions (blue) and a series of y ions (red), as well as ions corresponding to the loss of the lysine protecting groups in the fragmentation process (b and d). Ion peaks were assigned manually; along with precursor ion masses, these confirmed the incorporation of each ncAA at its expected position. The mass spectrometry analysis was performed three times with similar results. a, MS/MS spectra confirming PheI 5 incorporation at position 40. b, MS/MS spectra confirming BocK 1 incorporation at position 50. c, MS/MS spectra confirming NmH 2 incorporation at position 136. d, MS/MS spectra confirming CbzK 3 incorporation at position 150.

### DETAILED DESCRIPTION

The use of cell-based protein expression systems to produce exogenous proteins, particularly exogenous proteins comprising non-natural amino acids, can be challenging for several reasons. One issue is that the cell must also be able generate endogenous proteins that are essential for viability. For instance, if the protein expression system has been modified to allow the incorporation of non-natural amino acids into the exogenous protein, it can be desirable to avoid the incorporation of the non-natural amino acids into endogenous proteins. One approach to overcome this is to make use of systems comprising two ribosomes: a wild type ribosome for the production of proteins endogenous to the host cell and an orthogonal ribosome capable of translating orthogonal mRNAs encoding exogenous proteins.

Cell-based protein expression systems may include O-ribosomes for other reasons. Mutations to endogenous ribosomes can be toxic, and it has been found that some ribosomal mutations can be lethal to the cell even if present in just some copies of an endogenous ribosome (i.e. the mutations may be dominant lethal). However, O-ribosomes can tolerate these ribosomal mutations because, as discussed herein, they are isolated from the other functions of the host cell. Thus, the O-ribosome may be engineered for new desired functions. For instance, O-ribosomes can be evolved to decode new orthogonal codons (quadruplet codons, Neumann 2010) or new intrinsic polymerization functions (Schmied 2018).

However, as discussed in the background section, the yield of protein from expression systems comprising an O-ribosome can be low and un-optimized. In particular, the yield is not consistent when measured for different exogenous proteins.

Understanding of the factors that determine protein yield for natural translation is incomplete: a design of experiment study suggests that only half the variance in observed protein yield can be explained by known parameters²⁴. Nonetheless, the inventors noted that initiation of protein synthesis is commonly the rate limiting step of translation²⁵ and numerous studies suggest that RNA secondary structure in the 5′ UTR and the first 30 nt of the coding sequence are key determinants of translational initiation and protein yield^{24, 26} Indeed, thermodynamic models that predict the total free energy change (ΔGₜₒₜ(wt ribo)) from the free folded mRNA to a final ‘initiation competent’ state can be used to predict relative protein yields for natural translation^{27-29 (incorporated herein by reference)}. Previous work—varying 35 nt in the 5′ UTR immediately upstream of the start codon—indicates that protein yields for a given ORF (interpreted as reflecting the rate of translational initiation) are proportional to the equilibrium constant (i.e.: proportional to the log of the ΔGₜₒₜ (wt ribo)) for the formation of the initiation-competent state from the folded mRNA^{27, 30-33 (incorporated herein by reference)}. ΔGₜₒₜ (wt ribo) can be decomposed into mRNA unfolding (ΔG_{unfolding}) and binding of the wt-ribosome and tRNA^{fMet}_{CAU}, through base-pairing in the correct positions, to the mRNA (ΔG_{wt ribo binding}) (FIG. 1a).

Here, the inventors use a thermodynamic model of initiation and a simulated annealing optimization algorithm²⁷ to automate the discovery of 5′ UTR sequences for orthogonal translation of ORFs. The inventors also develop the algorithm to explicitly select for messages that bind O-ribosomes, but not other ribosomes, and increase the degrees of freedom in the search by exploring variation in both the 5′ UTR and the synonymous codons that encode amino acids, such as amino acids 2 to 12, of the ORF. Automating the discovery of O-mRNAs leads to sequences that provide up to 40-times more protein, and are up to 50-fold more orthogonal, than previous O-mRNAs; protein yields from the new O-mRNAs match or exceed those from WT mRNAs. These advances directly translate into a 33-fold increase in yield for incorporating three distinct ncAAs in response to an amber codon and two quadruplet codons using engineered triply orthogonal PylRS/tRNA^{Pyl} pairs.

Thus, in an aspect of the invention, there is provided a method of designing an mRNA which is an O-mRNA suitable for translation by an O-ribosome, wherein the mRNA comprises a 5′ UTR and an ORF, the method comprising:
(a) predicting the free energy difference between the free-folded state of the mRNA and the O-ribosome-bound initiation-competent state of the mRNA (ΔGₜₒₜ(O-ribo));
(b) introducing a modification into the 5′ UTR;
(c) predicting the new ΔGₜₒₜ(O-ribo) (ΔGₜₒₜ^{new}(O-ribo)) after modification;
(d) accepting the modification if said ΔGₜₒₜ^{new}(O-ribo) is more negative than the preceding ΔGₜₒₜ(O-ribo), and
accepting or rejecting the modification according to a probability distribution if said ΔGₜₒₜ^{new}(O-ribo) is more positive than the preceding ΔGₜₒₜ(O-ribo); and
(e) generating an O-mRNA sequence comprising the 5′ UTR which comprises the accepted modification(s).

An “O-ribosome” as used herein, is a ribosome that is less capable of translating, or is not capable of translating, mRNAs that are endogenous to a particular host cell compared to the endogenous ribosome; and which is capable of translating an mRNA which differs from the endogenous mRNAs (i.e. an O-mRNA).

An “O-mRNA” as used herein is a messenger RNA which would be less efficiently translated by a ribosome that is endogenous to a particular host cell compared to the translation of the endogenous mRNAs; and which is capable of being translated by a ribosome that differs from the endogenous ribosome (i.e. an O-ribosome).

The adjective “orthogonal” as used herein, describes components or features that are relevant to the O-ribosome and O-mRNA but not to the endogenous ribosome or mRNA. For instance, an orthogonal Shine Dalgarno sequence is associated with the O-mRNA as is capable of interacting with the orthogonal anti-Shine Dalgarno sequence of the O-ribosome.

An orthogonal Shine Dalgarno sequence would allow only reduced binding to the endogenous ribosome and an orthogonal anti-Shine Dalgarno sequence would allow only reduced binding to endogenous mRNAs.

As used herein, the O-ribosome and the O-mRNA function together. As such, the O-ribosome is capable of translating the O-mRNA.

In embodiments featuring more than one O-ribosome, a first set of O-mRNAs may be applicable to only one of the O-ribosomes, and a second set of O-mRNAs may be applicable to the other O-ribosome.

In an example, the O-ribosome may be an artificially altered or modified ribosome which differs from wild type ribosomes. The O-mRNA may be an mRNA that is not a substrate for a wild type ribosome.

The O-ribosome may comprise an altered 16S rRNA. In particular the 16 rRNA may be altered in a manner that affects the binding to a ribosome-binding site (RBS) of an mRNA.

The O-ribosome may comprise an altered anti-Shine Dalgarno sequence that is not capable, or is minimally capable, of binding to a wild type Shine Dalgarno sequence. In such instances, the O-mRNA comprises an altered RBS, for instance an altered Shine Dalgarno sequence, that is capable of binding to the O-ribosome.

In an embodiment, in the context of a host cell the O-ribosome does not synthesise, or minimally synthesises, the endogenous proteome. In such embodiments, the O-mRNA would not be translated by, or would minimally be translated by, the endogenous ribosome. The host cell is not particularly restricted and may be any host cell, particularly any host cell suitable for heterologous protein production. In some examples, the host cell is a prokaryotic cell, such as a bacterial cell. In particular, the host cell may be an E. coli cell.

In some examples, the O-ribosome may be 0-riboQ1. In addition, the O-ribosome may be any O-ribosome disclosed in WO2008/065398A1 or obtainable by a method disclosed in WO2008/065398A1. In addition, the O-ribosome may be any O-ribosome disclosed in WO2011/077075A1 or obtainable by a method disclosed in WO2011/077075A1. WO2008/065398A and WO2011/077075A1 are both incorporated herein by reference. The O-ribosome may be any O-ribosome disclosed in or obtainable by a method disclosed in any of Neumann, H et al. Nature 464, 441-444 (2010); Wang, K. et al. Nat. Biotechnol. 25, 770-777 (2007); or Schmied, W. H. et al. Nature 564, 444-448 (2018) (each of which is incorporated herein by reference).

The term “5′ UTR” is used herein according to its ordinary meaning in the art. In brief, a 5′ UTR is a region of an mRNA which is not translated into a polypeptide, is 5′ to the ORF, and is involved in recognition by the ribosome.

The term “ORF” is used herein according to its ordinary meaning in the art. In brief, the ORF is the part of the mRNA that is capable of being translated into an encoded protein.

The free-folded state of the mRNA is the state which exists when the mRNA is not bound to the ribosome and is free to form secondary structures.

The ribosome-bound initiation-competent state of the mRNA is the state that exists when the mRNA is bound to the ribosome, an initiator tRNA is bound, and the initiation of translation may begin.

In an embodiment, the modification is or comprises a single nucleotide change, insertion, or deletion introduced into the 5′ UTR.

During the method of the invention, the modification is accepted if said ΔGₜₒₜ^{new}(O-ribo) is more negative than the preceding ΔGₜₒₜ(O-ribo). The “preceding” ΔGₜₒₜ(O-ribo) is the ΔGₜₒₜ(O-ribo) predicted for the mRNA sequence before the modification is made. As discussed herein, the methods of the invention may be iterated, and so the preceding ΔGₜₒₜ(O-ribo) may be the ΔGₜₒₜ^{new}(O-ribo) calculated during the previous iteration.

The acceptance of a modification during the method of the invention means that the sequence alteration introduced by the modification is maintained for the next iteration of the method or, if the there is no further iteration of the method, is maintained in the sequence of the O-mRNA which is the output of the method.

During the method of the invention, the modification is accepted or rejected according to a probability distribution if said ΔGₜₒₜ^{new}(O-ribo) is more positive than the preceding ΔGₜₒₜ(O-ribo). The “preceding ΔGₜₒₜ(O-ribo)” is as discussed above. The probability distribution may be based upon conditional probability, wherein the chance of acceptance decreases as the difference between ΔGₜₒₜ^{new}(O-ribo) and ΔGₜₒₜ(O-ribo) increases. The probability may be a Monte-Carlo optimisation.

In an embodiment, the probability distribution according to which the modification is accepted or rejected is:

$$exp ( \frac{\vert Δ {G}_{tot}^{new} ( O - ribo ) - Δ {G}_{tot} ( O - ribo ) \vert}{{T}_{SA}} )$$

wherein T_{SA} is the simulated annealing temperature.

In a particular embodiment, the T_{SA} is adjusted to maintain at least a 0.1%, 1%, 2%, 3%, 4%, 5%, 6%, 7%, 8%, 9%, or 10% acceptance rate. In particular, the T_{SA} may be adjusted to maintain at least a 5% acceptance rate.

In a particular embodiment, the T_{SA} is adjusted to maintain an acceptance rate which is less than or equal to 75%, 50%, 40%, 30%, 25%, 20%, 15%, or 10%. In particular, the T_{SA} may be adjusted to maintain an acceptance rate which is less than or equal to 20%.

In a particular embodiment, the T_{SA} is adjusted to maintain a 0.1%-75%, 1%-50%, 2%-40%, 3%-30%, 4%-25%, or, in particular, a 5-20% acceptance rate.

The adjustment of the T_{SA} may mean that if the acceptance rate falls outside the aforementioned values for a certain number of iterations, the T_{SA} is increased or decreased to compensate. For instance, if the acceptance rate is below the lower threshold or above the upper threshold for 5, 10, 20, 30, 40, 50, 60, 70, 100, 200, or 500 iterations, the T_{SA} may be lowered or raised such that the acceptance rate is corrected. In a particular embodiment, the acceptance rate is considered for 50 iterations. In particular embodiments, the T_{SA} is adjusted by doubling or halving the value.

The rejection of a modification during the method of the invention means that the sequence alteration introduced by the modification is reversed and so not maintained for the next iteration of the method, or not maintained in the output sequence.

In some embodiments, the modification may be rejected if particular sequence constraints are violated. For instance, if the modified sequence would invalidate one of the assumptions of the underlying thermodynamic model then the modification may be rejected. Any step (d) of the methods of designing an O-mRNA disclosed herein may comprise the rejection of the modification based on these constraints, and this may be included in addition to the acceptance or rejection based on probability distributions as disclosed herein. The sequence constraints may be any as disclosed in Salis et al. (Nat. Biotechnol. 27, 946-950 (2009)), which is incorporated by reference.

As an example of such a constraint, in an embodiment if the energy required to unfold the 16S rRNA binding site on the mRNA sequence is above a particular threshold, such as >6 kcal/mol, the modification is rejected. Alternatively or in addition, the presence of long-range nucleotide interactions may be quantified and the modification may be rejected if particular conditions are not met. For instance, if the equilibrium probability of nucleotides i and j forming a base pair in solution is considered to be proportional to P=|i−j|^{-1.44}, and for each base pair in sequence S, P is calculated, the modification may be rejected if the minimum p is <6×10⁻³. As another example of a constraint, which may be included as an alternative or in addition to any of the other constraints, the creation of new AUG or GUG start codons within the ribosome binding sequence may be disallowed, and so any modifications introducing said codons may be rejected.

In all embodiments disclosed herein where a modification is accepted or rejected according to a probability distribution, as an alternative the modification may simply be rejected. As such, in an embodiment if said ΔGₜₒₜ^{new}(O-ribo) is more negative than the preceding ΔGₜₒₜ(O-ribo) the modification is rejected. This is also applicable to the further embodiments disclosed herein.

The generation of the O-mRNA sequence means that a final sequence is output which includes the cumulative effect of all of the accepted modifications.

In an embodiment, the first round of the method of the invention is performed on a potential mRNA sequence with a randomly generated 5′ UTR. The length of the 5′ UTR is not particularly limited. During the method, the length of the 5′ UTR and may increase or decrease due to insertion or deletion modifications. In particular embodiments, the initial 5′ UTR is from 30 to 40 nucleotides long, or in particular is 35 nucleotides. Alternatively, the 5′ UTR may be longer but a 30-40, or in particular 35, nucleotide window is considered by the methods of the invention for modification. The 35-nucleotide window may be the 35 nucleotides of the 5′ UTR that are closest to the start codon. In other embodiments, the initial 5′ UTR may be shorter, such as a 15, 20, or 25 nucleotide 5′ UTR, or longer, such as at least 40, 50, or more nucleotides. It might be desirable to generate a 5′ UTR which is of a particular length, in which case a 15, 20, 25, 30, 35, 45, 50 nucleotide window may be considered such that a particular length of output sequence may be achieved.

The 5′ UTR to which step (a) is applied may comprise a wild type Shine Dalgarno sequence, or the five-nucleotide core of a wild type Shine Dalgarno sequence. In other embodiments, the 5′ UTR to which step (a) is applied may comprise an orthogonal Shine Dalgarno sequence, as discussed herein. The 5′ UTR may be of a random sequence apart from the Shine Dalgarno sequence. The Shine Dalgarno sequence may be five nucleotides from the start codon of the ORF, which is predicted to be the optimal spacing.

The methods of the invention require the prediction of the ΔGₜₒₜ(O-ribo). A method for the prediction of this value is described in detail in the Examples section. In an embodiment, ΔGₜₒₜ(O-ribo) is the sum of the free energy required to unfold the mRNA (ΔG_{unfolding}) and the free energy released upon the mRNA binding to the O-ribosome to form the O-ribosome-bound initiation-competent state (ΔG_{o-ribo binding}).

In an embodiment, the ΔGₜₒₜ(O-ribo) may be calculated as follows:

$$Δ {G}_{tot} ( O - ribo ) = ( Δ {G}_{mRNA - O - rRNA} + Δ {G}_{start} + Δ {G}_{spacing} - Δ {G}_{standby} ) + Δ {G}_{unfolding} ;$$

wherein
ΔG_{mRNA-O-rRNA} is the free energy of the predicted co-folded secondary structure of the last 9 nucleotides of the orthogonal 16S rRNA and the mRNA;
ΔGₛₜₐᵣₜ is the energy released from binding of an initiator tRNA to the start codon of the ORF;
ΔG_{spacing} is an energy penalty for non-optimal spacing length between the Shine Dalgarno sequence and the start codon;
ΔG_{standby} is the energy required to unfold secondary structures that sequester the four nucleotides upstream of the Shine Dalgarno sequence; and
ΔG_{unfolding} is the energy required to unfold secondary structures in the mRNA.

In a particular embodiment, the above values are calculated as disclosed in Salis et al. (Nat. Biotechnol. 27, 946-950 (2009)), which is incorporated by reference. For instance, ΔG_{spacing} may be calculated as disclosed in section 3 of the Supplementary Methods of this publication.

The method of the invention may be iterated such that a plurality of modifications are considered for acceptance or rejection and the final output sequence includes the cumulative effect of all of said accepted modifications. In particular, steps (b) to (d) of the method of the invention may be iterated. In an embodiment, the method is iterated at least 200, 300, 400, 500, 1000, 5000, or, in particular, 10000 times. In other embodiments, the method may be iterated until consecutive iterations do not lead to a more negative ΔGₜₒₜ^{new}(O-ribo), as disclosed herein.

In a particular embodiment, there is provided a method of designing an mRNA which is an O-mRNA suitable for translation by an O-ribosome, wherein the mRNA comprises a 5′ UTR and an ORF, the method comprising:
(a) predicting the free energy difference between the free-folded state of the mRNA and the O-ribosome-bound initiation-competent state of the mRNA (ΔGₜₒₜ(O-ribo)), wherein the 5′ UTR comprises a wild type Shine Dalgarno sequence;
(b) introducing a modification which is or which comprises a single nucleotide change, insertion, or deletion into the 5′ UTR;
(c) predicting the new ΔGₜₒₜ(O-ribo) (ΔGₜₒₜ^{new}(O-ribo)) after modification;
(d) accepting the modification if said ΔGₜₒₜ^{new}(O-ribo) is more negative than the preceding ΔGₜₒₜ(O-ribo), and
accepting or rejecting the modification according to a probability distribution if said ΔGₜₒₜ^{new}(O-ribo) is more positive than the preceding ΔGₜₒₜ(O-ribo); wherein the magnitude of the difference between said ΔGₜₒₜ^{new}(O-ribo) and said ΔGₜₒₜ(O-ribo) determines the probability of acceptance, wherein a smaller magnitude is associated with a higher chance of acceptance compared to a larger magnitude; and
(e) iterating steps (b) to (d) at least 500, 1000, 5000, or, in particular, 10000 times, and then
   generating an O-mRNA sequence comprising the 5′ UTR which comprises the accepted modification(s).

In a particular embodiment, there is provided a method of designing an mRNA which is an O-mRNA suitable for translation by an O-ribosome, wherein the mRNA comprises a 5′ UTR and an ORF, the method comprising:
(a) predicting the free energy difference between the free-folded state of the mRNA and the O-ribosome-bound initiation-competent state of the mRNA (ΔGₜₒₜ(O-ribo)), wherein the 5′ UTR comprises a wild type Shine Dalgarno sequence;
(b) introducing a modification which comprises a single nucleotide change, insertion, or deletion at any one of the 35 nucleotides of the 5′ UTR that are closest to the ORF;
(c) predicting the new ΔGₜₒₜ(O-ribo) (ΔGₜₒₜ^{new}(O-ribo)) after modification;
(d) accepting the modification if said ΔGₜₒₜ^{new}(O-ribo) is more negative than the preceding ΔGₜₒₜ(O-ribo), and
accepting or rejecting the modification according to

$$exp ( \frac{\vert Δ {G}_{tot}^{new} ( O - ribo ) - Δ {G}_{tot} ( O - ribo ) \vert}{{T}_{SA}} )$$

if said ΔGₜₒₜ^{new}(O-ribo) is more positive than the preceding ΔGₜₒₜ(O-ribo); and
(e) iterating steps (b) to (d) at least 500, 1000, 5000, or, in particular, 10000 times, and then
   generating an O-mRNA sequence comprising the 5′ UTR which comprises the accepted modification(s).

In some embodiments, the method of the invention may comprise optimising the O-mRNA such that the efficiency of translation by the O-ribosome is increased and the efficiency of translation by a second ribosome (2^{nd}-ribosome) is decreased.

Thus, in an additional embodiment, the method of the invention is a method of designing an mRNA which is an O-mRNA suitable for translation by an O-ribosome in a cell also comprising a second ribosome (2^{nd}-ribosome), wherein the mRNA comprises a 5′ UTR and an ORF, the method comprising:
(a) predicting the free energy difference between the free-folded state of the mRNA and the O-ribosome-bound initiation-competent state of the O-mRNA (ΔGₜₒₜ(O-ribo)) and predicting the free energy difference between the free-folded state of the mRNA and the 2^{nd}-ribosome-bound initiation-competent state of the mRNA (ΔGₜₒₜ(2^{nd}-ribo));
(b) introducing a modification into the 5′ UTR;
(c) predicting the new ΔGₜₒₜ(O-ribo) (ΔGₜₒₜ^{new}(O-ribo)) and the new ΔGₜₒₜ(2^{nd}-ribo) (ΔGₜₒₜ^{new}(2^{nd}-ribo) after modification;
(d) accepting the modification if said ΔGₜₒₜ^{new}(O-ribo) is more negative than the preceding ΔGₜₒₜ(O-ribo) and said ΔGₜₒₜ^{new}(2^{nd}-ribo) is more positive than the preceding ΔGₜₒₜ(2^{nd}-ribo), and
accepting or rejecting the modification according to a probability distribution if said ΔGₜₒₜ^{new}(O-ribo) is more positive than the preceding ΔGₜₒₜ(O-ribo) or if said ΔGₜₒₜ^{new}(2^{nd}-ribo) is more negative than the preceding ΔGₜₒₜ(2^{nd}-ribo); and
(e) generating an O-mRNA sequence comprising the 5′ UTR which comprises the accepted modification(s).

The 5′ UTR sequence of step (a) may comprise a Shine Dalgarno sequence that is predicted to be perfectly complementary to the anti-Shine Dalgarno sequence of the O-ribosome for which increased translation of the O-mRNA is being optimised. This Shine Dalgarno sequence is referred to as an orthogonal Shine Dalgarno sequence (O-SD). The 5′ UTR sequence of step (a) may comprise a five-nucleotide core of an O-SD. In an embodiment, the O-SD is five nucleotides from the start codon of the ORF. In an embodiment, the modification is not introduced into the five-nucleotide core of the O-SD. For instance, the O-SD may be TAATCCCAT and the modification is not introduced into the TCCCA. In some embodiments, the modification is not introduced into the O-SD. In other embodiments, the 5′ UTR sequence may comprise a wild type Shine Dalgarno sequence.

The first round of the method of the invention may be performed on a potential mRNA sequence with a randomly generated 5′ UTR. The initial length, final length, or length of window of nucleotides to be considered may be any disclosed herein. The 5′ UTR may be of a random sequence apart from the Shine Dalgarno sequence. The Shine Dalgarno sequence may be five nucleotides from the start codon of the ORF, which is predicted to be the optimal spacing.

The 2^{nd}-ribosome may be a wild type ribosome (“WT-ribosome). A “WT-ribosome” as used herein, is a ribosome that is capable of translating the endogenous mRNAs within the intended host cell and which is less capable of translating, or is not capable of translating, the O-mRNA. For example, the WT-ribosome may comprise a wild type region for interacting with the RBS of an mRNA. The 16S rRNA of the WT-ribosome (referred to as the wild type 16S rRNA) may comprise a wild type sequence. In particular, the WT-ribosome may comprise a wild type anti-Shine Dalgarno sequence. In particular examples, all components of the WT-ribosome may be wild type.

Alternatively, the 2^{nd}-ribosome may be another O-ribosome. For instance, the 2^{nd}-ribosome may be an O-ribosome comprising a second orthogonal anti-Shine Dalgarno sequence which differs from the orthogonal anti-Shine Dalgarno sequence of the first ribosome (i.e. the ribosome for which increased translation of the mRNA is being optimised). The second O-ribosome may efficiency translate a set of O-mRNAs which differ from the O-mRNAs that are efficiently translated by the first ribosome.

A method for the prediction of the ΔGₜₒₜ(2^{nd}-ribo) value is described in detail in the Examples section. In an embodiment, ΔGₜₒₜ(2^{nd}-ribo) is the sum of the free energy required to unfold the mRNA (ΔG_{unfolding}) and the free energy released upon the mRNA binding to the 2^{nd}-ribosome to form the 2^{nd}-ribosome-bound initiation-competent state (ΔG_{2nd-ribo binding}).

In an embodiment, the ΔGₜₒₜ(2^{nd}-ribo) may be calculated as follows:

$$Δ {G}_{tot} ( {2}^{nd} - ribo ) = ( Δ {G}_{mRNA - 2 nd - rRNA} + Δ {G}_{start} + Δ {G}_{spacing} - Δ {G}_{standby} ) + Δ {G}_{unfolding} ;$$

ΔG_{mRNA-2nd-rRNA} is the free energy of the predicted co-folded secondary structure of the last 9 nucleotides of the 16S rRNA and the mRNA;
ΔGₛₜₐᵣₜ is the energy released from binding of an initiator tRNA to the start codon of the ORF;
ΔG_{spacing} is an energy penalty for non-optimal spacing length between the Shine Dalgarno sequence and the start codon;
ΔG_{standby} is the energy required to unfold secondary structures that sequester the four nucleotides upstream of the Shine Dalgarno sequence; and
ΔG_{unfolding} is the energy required to unfold secondary structures in the mRNA.

The above calculation may be performed as discussed for ΔGₜₒₜ(O-ribo).

In an embodiment, the modification is or comprises a single nucleotide change, insertion, or deletion introduced into the 5′ UTR.

The modification is accepted or rejected according to a probability distribution if said ΔGₜₒₜ^{new}(O-ribo) is more positive than the preceding ΔGₜₒₜ(O-ribo) or if said ΔGₜₒₜ^{new}(2^{nd}-ribo) is more negative than the preceding ΔGₜₒₜ(2^{nd}-ribo). The “preceding ΔGₜₒₜ(O-ribo)” is as discussed above and the “preceding ΔGₜₒₜ(2^{nd}-ribo)” should be interpreted in the same manner. The probability distribution may be based upon conditional probability, wherein the chance of acceptance decreases as the difference between ΔGₜₒₜ^{new}(O-ribo) and ΔGₜₒₜ(O-ribo) increases or the difference between ΔGₜₒₜ^{new}(2^{nd}-ribo) and ΔGₜₒₜ(2^{nd}-ribo) increases. The probability may be a Monte-Carlo optimisation.

The method of the invention may be iterated such that a plurality of modifications are considered for acceptance or rejection and the final output sequence includes the cumulative effect of all of said accepted modifications. In particular, steps (b) to (d) of the method of the invention may be iterated. The method may be iterated until at least 10, 50, 100, 250, 500, 1000, 2000, 3000, 5000, or 10000 consecutive iterations do not lead to a more negative ΔGₜₒₜ^{new}(O-ribo) or a more positive ΔGₜₒₜ^{new}(2^{nd}-ribo). Alternatively, the method may be iterated a set number of times, as disclosed herein.

In a particular embodiment, the method of the invention is a method of designing an mRNA which is an O-mRNA suitable for translation by an O-ribosome in a cell also comprising a 2^{nd}-ribosome, wherein the mRNA comprises a 5′ UTR and an ORF, the method comprising:
(a) predicting the free energy difference between the free-folded state of the mRNA and the O-ribosome-bound initiation-competent state of the O-mRNA (ΔGₜₒₜ(O-ribo)) and predicting the free energy difference between the free-folded state of the mRNA and the 2^{nd}-ribosome-bound initiation-competent state of the mRNA (ΔGₜₒₜ(2^{nd}-ribo)), wherein the 5′ UTR comprises an O-SD;
(b) introducing a modification which is or which comprises a single nucleotide change, insertion, or deletion into the 5′ UTR, wherein the modification is not introduced into the O-SD five-nucleotide core;
(c) predicting the new ΔGₜₒₜ(O-ribo) (ΔGₜₒₜ^{new}(O-ribo)) and the new ΔGₜₒₜ(2^{nd}-ribo) (ΔGₜₒₜ^{new}(2^{nd}-ribo) after modification;
(d) accepting the modification if said ΔGₜₒₜ^{new}(O-ribo) is more negative than the preceding ΔGₜₒₜ(O-ribo) and said ΔGₜₒₜ^{new}(2^{nd}-ribo) is more positive than the preceding ΔGₜₒₜ(2^{nd}-ribo), and
accepting or rejecting the modification according to a probability distribution if said ΔGₜₒₜ^{new}(O-ribo) is more positive than the preceding ΔGₜₒₜ(O-ribo) or if said ΔGₜₒₜ^{new}(2^{nd}-ribo) is more negative than the preceding ΔGₜₒₜ(2^{nd}-ribo); wherein the magnitude of the difference between said ΔGₜₒₜ^{new}(O-ribo) and said ΔGₜₒₜ(O-ribo) or between said ΔGₜₒₜ^{new}(2^{nd}-ribo) and said ΔGₜₒₜ(2^{nd}-ribo) determines the probability of acceptance, wherein a smaller magnitude is associated with a higher chance of acceptance compared to a larger magnitude; and
(e) iterating steps (b) to (d) until at least 10, 50, 100, 250, or, in particular, 500 consecutive iterations do not lead to a more negative ΔGₜₒₜ^{new}(O-ribo) or a more positive ΔGₜₒₜ^{new}(2^{nd}-ribo); and
   generating an O-mRNA sequence comprising the 5′ UTR which comprises the accepted modification(s).

In a particular embodiment, the method of the invention is a method of designing an mRNA which is an O-mRNA suitable for translation by an O-ribosome in a cell also comprising a 2^{nd}-ribosome, wherein the mRNA comprises a 5′ UTR and an ORF, the method comprising:
(a) predicting the free energy difference between the free-folded state of the mRNA and the O-ribosome-bound initiation-competent state of the mRNA (ΔGₜₒₜ(O-ribo)), predicting the free energy difference between the free-folded state of the mRNA and the 2^{nd}-ribosome-bound initiation-competent state of the mRNA (ΔGₜₒₜ(2^{nd}-ribo)), and calculating ΔGₜₒₜ(opt) according to the formula: ΔGₜₒₜ(opt)=ΔGₜₒₜ(O-ribo)−X*ΔGₜₒₜ(2^{nd}-ribo);
(b) introducing a modification which comprises a single nucleotide change, insertion, or deletion into the 5′ UTR;
(c) predicting the new ΔGₜₒₜ(O-ribo) (ΔGₜₒₜ^{new}(O-ribo)) and the new ΔGₜₒₜ(2^{nd}-ribo) (ΔGₜₒₜ^{new}(2^{nd}-ribo) after modification, and calculating ΔGₜₒₜ^{new}(opt) according to the formula: ΔGₜₒₜ^{new}(opt)=ΔGₜₒₜ^{new}(O-ribo)−X*ΔGₜₒₜ^{new}(2^{nd}-ribo);
(d) accepting the modification if said ΔGₜₒₜ^{new}(opt) is more negative than the preceding ΔGₜₒₜ(opt), and
accepting or rejecting the modification according to a probability distribution if said ΔGₜₒₜ^{new}(opt) is more positive than the preceding ΔGₜₒₜ(opt);
(e) generating an O-mRNA sequence comprising the 5′ UTR which comprises the accepted modification(s).

In some embodiments, X is a number from 0.1 to 2, in particular 0.5. In other examples, X may be a number from 0.1 to 2, 0.15 to 1.5, 0.2 to 1, 0.25 to 0.9, 0.3 to 0.8, 0.35 to 0.7, 0.4 to 0.6, 0.45 to 0.55, or 0.5. As the skilled person would understand, the weighting may be applied to ΔGₜₒₜ^{new}(O-ribo) for the same result, and this is encompassed by the above formula. The weighting may be adjusted to prioritise a particular property, for instance a higher X would prioritise the minimisation of translation by the 2^{nd}-ribsome whereas a lower X would prioritise the maximisation of translation by the first ribosome (i.e. the O-ribosome for which the O-mRNA is intended).

The modification is accepted if said ΔGₜₒₜ^{new}(opt) is more negative than the preceding ΔGₜₒₜ(opt). The “preceding” ΔGₜₒₜ(opt) is the ΔGₜₒₜ(opt) predicted for the mRNA sequence before the modification is made. As discussed herein, the methods of the invention may be iterated, and so the preceding ΔGₜₒₜ(opt) may be the ΔGₜₒₜ^{new}(opt) calculated during the previous iteration.

The modification is accepted or rejected according to a probability distribution if said ΔGₜₒₜ^{new}(opt) is more positive than the preceding ΔGₜₒₜ(opt). The “preceding ΔGₜₒₜ(opt)” is as discussed above. The probability distribution may be based upon conditional probability, wherein the chance of acceptance decreases as the difference between ΔGₜₒₜ^{new}(opt) and ΔGₜₒₜ(opt) increases. The probability may be a Monte-Carlo optimisation.

In an embodiment, the probability distribution according to which the modification is accepted or rejected is:

$$exp ( \frac{Δ {G}_{tot}^{new} ( opt ) - Δ {G}_{tot} ( opt )}{{T}_{SA}} )$$

The T_{SA} may be adjusted in any manner as disclosed herein. In a particular embodiment, the T_{SA} is adjusted to maintain a 5-20% acceptance rate.

In some embodiments, the modification may be rejected if particular sequence constraints are violated, as discussed herein. In addition, or as an alternative, to the constraints already discussed, the modification may be rejected if a second O-SD or second O-SD core is introduced into the sequence. This is to prevent initiation from the wrong site. For instance, if the sequence ‘TCCCA’ (an example of an O-SD core) is introduced, the modification may be rejected.

The method of the invention may be iterated such that a plurality of modifications are considered for acceptance or rejection and the final output sequence includes the cumulative effect of all of said accepted modifications. In particular, steps (b) to (d) of the method of the invention may be iterated. The method may be iterated until at least 10, 50, 100, 250, 500, 1000, 2000, 3000, 5000, or 10000 consecutive iterations do not lead to a more negative ΔGₜₒₜ^{new}(opt). In other embodiments, the method may be iterated a set number of times, as discussed herein.

In a particular embodiment, there is provided a method of designing an mRNA which is an O-mRNA suitable for translation by an O-ribosome in a cell also comprising a 2^{nd}-ribosome, wherein the mRNA comprises a 5′ UTR and an ORF, the method comprising:
(a) predicting the free energy difference between the free-folded state of the mRNA and the O-ribosome-bound initiation-competent state of the mRNA (ΔGₜₒₜ(O-ribo)), predicting the free energy difference between the free-folded state of the mRNA and the 2^{nd}-ribosome-bound initiation-competent state of the mRNA (ΔGₜₒₜ(2^{nd}-ribo)), and calculating ΔGₜₒₜ(opt) according to the formula: ΔGₜₒₜ(opt)=ΔGₜₒₜ(O-ribo)−X*ΔGₜₒₜ(2^{nd}-ribo); wherein the 5′ UTR comprises an O-SD;
(b) introducing a modification into the 5′ UTR, wherein the modification is not introduced into the O-SD five-nucleotide core;
(c) predicting the new ΔGₜₒₜ(O-ribo) (ΔGₜₒₜ^{new}(O-ribo)) and the new ΔGₜₒₜ(2^{nd}-ribo) (ΔGₜₒₜ^{new}(2^{nd}-ribo) after modification, and calculating ΔGₜₒₜ^{new}(opt) according to the formula: ΔGₜₒₜ^{new}(opt)=ΔGₜₒₜ^{new}(O-ribo)−X*ΔGₜₒₜ^{new}(2^{nd}-ribo);
(d) accepting the modification if said ΔGₜₒₜ^{new}(opt) is more negative than the preceding ΔGₜₒₜ(opt), and
accepting or rejecting the modification according to a probability distribution if said ΔGₜₒₜ^{new}(opt) is more positive than the preceding ΔGₜₒₜ(opt); wherein the magnitude of the difference between said ΔGₜₒₜ^{new}(opt) and said ΔGₜₒₜ(opt) determines the probability of acceptance, wherein a smaller magnitude is associated with a higher chance of acceptance compared to a larger magnitude; and
(e) iterating steps (b) to (d) until at least 10, 50, 100, 250, or, in particular, 500 consecutive iterations do not lead to a more negative ΔGₜₒₜ^{new}(opt), and then
generating an O-mRNA sequence comprising the 5′ UTR which comprises the accepted modification(s);
wherein X is a number from 0.1 to 2, in particular 0.5.

In a particular embodiment, there is provided a method of designing an mRNA which is an O-mRNA suitable for translation by an O-ribosome in a cell also comprising a 2^{nd}-ribosome, wherein the mRNA comprises a 5′ UTR and an ORF, the method comprising:
(a) predicting the free energy difference between the free-folded state of the mRNA and the O-ribosome-bound initiation-competent state of the mRNA (ΔGₜₒₜ(O-ribo)), predicting the free energy difference between the free-folded state of the mRNA and the 2^{nd}-ribosome-bound initiation-competent state of the mRNA (ΔGₜₒₜ(2^{nd}-ribo)), and calculating ΔGₜₒₜ(opt) according to the formula: ΔGₜₒₜ(opt)=ΔGₜₒₜ(O-ribo)−X*ΔGₜₒₜ(2^{nd}-ribo); wherein the 5′ UTR comprises an O-SD;
(b) introducing a modification which is a single nucleotide change, insertion, or deletion into the 5′ UTR, wherein the modification is not introduced into the O-SD five-nucleotide core;
(c) predicting the new ΔGₜₒₜ(O-ribo) (ΔGₜₒₜ^{new}(O-ribo)) and the new ΔGₜₒₜ(2^{nd}-ribo) (ΔGₜₒₜ^{new}(2^{nd}-ribo) after modification, and calculating ΔGₜₒₜ^{new}(opt) according to the formula: ΔGₜₒₜ^{new}(opt)=ΔGₜₒₜ^{new}(O-ribo)−X*ΔGₜₒₜ^{new}(2^{nd}-ribo);
(d) accepting the modification if said ΔGₜₒₜ^{new}(opt) is more negative than the preceding ΔGₜₒₜ(opt), and
accepting or rejecting the modification according to

$$exp ( \frac{Δ {G}_{tot}^{new} ( opt ) - Δ {G}_{tot} ( opt )}{{T}_{SA}} )$$

if said ΔGₜₒₜ^{new}W(opt) is more positive than the preceding ΔGₜₒₜ(opt);
(e) iterating steps (b) to (d) until at least 10, 50, 100, 250, or, in particular, 500 consecutive iterations do not lead to a more negative ΔGₜₒₜ^{new}(opt), and then
generating an O-mRNA sequence comprising the 5′ UTR which comprises the accepted modification(s);
wherein X is a number from 0.1 to 2, in particular 0.5.

The methods of designing an O-mRNA may comprise optimising the O-mRNA such that the efficiency of translation by a first O-ribosome is increased and the efficiency of translation by a second O-ribosome and by a WT-ribosome is decreased.

Such methods are as disclosed above, wherein the free energy difference between the free-folded state of the mRNA and ribosome-bound initiation-competent state is predicted for each of the ribosomes. As discussed, this predication is made before and after the introduction of a modification to the mRNA sequence. In embodiments comprising more than two ribosomes, the modification may be accepted if the ΔGₜₒₜ becomes more negative for the first ribosome (i.e. the ribosome for which translation efficiency is increased) and more positive for the other ribosomes. If the ΔGₜₒₜ values are not all altered favourably, the modification may be accepted or rejected according to a probability distribution as disclosed herein. The ΔGₜₒₜ values may be combined to form a single value which is considered for acceptance or rejection. For instance, the ΔGₜₒₜ values may be combined according to the following formula ΔGₜₒₜ(opt)=X*ΔGₜₒₜ(1^{st}-O-ribo)−Y*ΔGₜₒₜ(WT-ribo)−Z*ΔGₜₒₜ(2^{nd}-O-ribo), wherein X, Y, and Z are weightings. These weightings may be adjusted to prioritise a particular property (e.g. optimisation of translation by the first O-ribosome or decrease in translation by the second O-ribosome). ΔGₜₒₜ(opt) may be considered for acceptance or rejection as disclosed herein.

As will be apparent to the skilled person, the above may be adapted such that the efficiency of translation by a first O-ribosome is increased and the efficiency of translation by two, three, four, or more other ribosomes is decreased. The same or different weightings may be associated with the ΔGₜₒₜ values for each of the ribosomes for which the efficiency of translation is decreased. The ΔGₜₒₜ(1^{st}-O-ribo) may also be associated with a weighting.

The inventors have further identified that replacing codons within the ORF with synonymous codons can lead to an improved O-mRNA. Synonymous codons are those that encode the same amino acid, and hence the replacement of a sense codon with a synonym does not alter the sequence of the encoded protein. As such, in an embodiment, the modification of step (b) may comprise the exchange of any one of codons 2 to 20, 2 to 15, 2 to 12, 2 to 10, or 2 to 5, within the ORF with a synonymous codon. In a particular embodiment, the modification of step (b) may comprise the exchange of any one of codons 2 to 12 within the ORF with a synonymous codon.

The exchange of a codon for a synonym may be an alternative to the introduction of a single nucleotide change, insertion, or deletion into the 5′ UTR. As such, step (b) may comprise introducing a modification which is a single nucleotide change, insertion, or deletion into the 5′ UTR, or the exchange of any one of codons 2 to 20, 2 to 15, 2 to 12, 2 to 10, or 2 to 5 (in particular 2 to 12) within the ORF with a synonymous codon.

In such embodiments, the generated O-mRNA sequence comprises the 5′ UTR and the ORF which comprise the accepted modification(s).

Thus, in an additional embodiment, the method of the invention is a method of designing an mRNA which is an O-mRNA suitable for translation by an O-ribosome, wherein the mRNA comprises a 5′ UTR and an ORF, the method comprising:
(a) predicting the free energy difference between the free-folded state of the mRNA and the O-ribosome-bound initiation-competent state of the O-mRNA (ΔGₜₒₜ(O-ribo));
(b) introducing a modification which is or comprises a single nucleotide change, insertion, or deletion into the 5′ UTR, or the exchange of any one of codons 2 to 20, 2 to 15, 2 to 12, 2 to 10, or 2 to 5 within the ORF with a synonymous codon;
(c) predicting the new ΔGₜₒₜ(O-ribo) (ΔGₜₒₜ^{new}(O-ribo)) after modification;
(d) accepting the modification if said ΔGₜₒₜ^{new}(O-ribo) is more negative than the preceding ΔGₜₒₜ(O-ribo), and
accepting or rejecting the modification according to a probability distribution if said ΔGₜₒₜ^{new}(O-ribo) is more positive than the preceding ΔGₜₒₜ(O-ribo); and
(e) generating an O-mRNA sequence comprising the 5′ UTR and the ORF which comprise the accepted modification(s).

In an additional embodiment, the method of the invention is a method of designing an mRNA which is an O-mRNA suitable for translation by an O-ribosome in a cell also comprising a 2^{nd}-ribosome, wherein the mRNA comprises a 5′ UTR and an ORF, the method comprising:
(a) predicting the free energy difference between the free-folded state of the mRNA and the O-ribosome-bound initiation-competent state of the O-mRNA (ΔGₜₒₜ(O-ribo)) and predicting the free energy difference between the free-folded state of the mRNA and the 2^{nd}-ribosome-bound initiation-competent state of the mRNA (ΔGₜₒₜ(2^{nd}-ribo));
(b) introducing a modification which is or comprises a single nucleotide change, insertion, or deletion into the 5′ UTR, or the exchange of any one of codons 2 to 20, 2 to 15, 2 to 12, 2 to 10, or 2 to 5 within the ORF with a synonymous codon;
(c) predicting the new ΔGₜₒ(O-ribo) (ΔGₜₒₜ^{new}(O-ribo)) and the new ΔGₜₒₜ(2^{nd}-ribo) (ΔGₜₒₜ^{new}(2^{nd}-ribo) after modification;
(d) accepting the modification if said ΔGₜₒₜ^{new}(O-ribo) is more negative than the preceding ΔGₜₒₜ(O-ribo) and said ΔGₜₒₜ^{new}(2^{nd}-ribo) is more positive than the preceding ΔGₜₒₜ(2^{nd}-ribo), and
accepting or rejecting the modification according to a probability distribution if said ΔGₜₒₜ^{new}(O-ribo) is more positive than the preceding ΔGₜₒₜ(O-ribo) or if said ΔGₜₒₜ^{new}(2^{nd}-ribo) is more negative than the preceding ΔGₜₒₜ(2^{nd}-ribo); and
(e) generating an O-mRNA sequence comprising the 5′ UTR and the ORF which comprise the accepted modification(s).

In a particular embodiment, the method of the invention is a method of designing an mRNA which is an O-mRNA suitable for translation by an O-ribosome in a cell also comprising a 2^{nd}-ribosome, wherein the mRNA comprises a 5′ UTR and an ORF, the method comprising:
(a) predicting the free energy difference between the free-folded state of the mRNA and the O-ribosome-bound initiation-competent state of the O-mRNA (ΔGₜₒₜ(O-ribo)) and predicting the free energy difference between the free-folded state of the mRNA and the 2^{nd}-ribosome-bound initiation-competent state of the mRNA (ΔGₜₒₜ(2^{nd}-ribo));
(b) introducing a modification which is or comprises a single nucleotide change, insertion, or deletion into the 5′ UTR, or the exchange of any one of codons 2 to 12 within the ORF with a synonymous codon;
(c) predicting the new ΔGₜₒₜ(O-ribo) (ΔGₜₒₜ^{new}(O-ribo)) and the new ΔGₜₒₜ(2^{nd}-ribo) (ΔGₜₒₜ^{new}(2^{nd}-ribo) after modification;
(d) accepting the modification if said ΔGₜₒₜ^{new}(O-ribo) is more negative than the preceding ΔGₜₒₜ(O-ribo) and said ΔGₜₒₜ^{new}(2^{nd}-ribo) is more positive than the preceding ΔGₜₒₜ(2^{nd}-ribo), and
accepting or rejecting the modification according to a probability distribution if said ΔGₜₒₜ^{new}(O-ribo) is more positive than the preceding ΔGₜₒₜ(O-ribo) or if said ΔGₜₒₜ^{new}(2^{nd}-ribo) is more negative than the preceding ΔGₜₒₜ(2^{nd}-ribo); wherein the magnitude of the difference between said ΔGₜₒₜ^{new}(O-ribo) and said ΔGₜₒₜ(O-ribo) or between said ΔGₜₒₜ^{new}(2^{nd}-ribo) and said ΔGₜₒₜ(2^{nd}-ribo) determines the probability of acceptance, wherein a smaller magnitude is associated with a higher chance of acceptance compared to a larger magnitude; and
(e) generating an O-mRNA sequence comprising the 5′ UTR and the ORF which comprise the accepted modification(s).

In another embodiment, the method of the invention is a method of designing an mRNA which is an O-mRNA suitable for translation by an O-ribosome in a cell also comprising a 2^{nd}-ribosome, wherein the mRNA comprises a 5′ UTR and an ORF, the method comprising:
(a) predicting the free energy difference between the free-folded state of the mRNA and the O-ribosome-bound initiation-competent state of the mRNA (ΔGₜₒₜ(O-ribo)), predicting the free energy difference between the free-folded state of the mRNA and the 2^{nd}-ribosome-bound initiation-competent state of the mRNA (ΔGₜₒₜ(2^{nd}-ribo)), and calculating ΔGₜₒₜ(opt) according to the formula: ΔGₜₒₜ(opt)=ΔGₜₒₜ(O-ribo)−X*ΔGₜₒₜ(2^{nd}-ribo);
(b) introducing a modification which is or comprises a single nucleotide change, insertion, or deletion into the 5′ UTR, or the exchange of any one of codons 2 to 20, 2 to 15, 2 to 12, 2 to 10, or 2 to 5 within the ORF with a synonymous codon;
(c) predicting the new ΔGₜₒₜ(O-ribo) (ΔGₜₒₜ^{new}(O-ribo)) and the new ΔGₜₒₜ(2^{nd}-ribo) (ΔGₜₒₜ^{new}(2^{nd}-ribo)) after modification, and calculating ΔGₜₒₜ^{new}(opt) according to the formula: ΔGₜₒₜ^{new}(opt)=ΔGₜₒₜ^{new}(O-ribo)−X*ΔGₜₒₜ^{new}(2^{nd}-ribo);
(d) accepting the modification if said ΔGₜₒₜ^{new}(opt) is more negative than the preceding ΔGₜₒₜ(opt), and
accepting or rejecting the modification according to a probability distribution if said ΔGₜₒₜ^{new}(opt) is more positive than the preceding ΔGₜₒₜ(opt);
(e) generating an O-mRNA sequence comprising the 5′ UTR and the ORF which comprise the accepted modification(s). Optionally, wherein X is a number as disclosed herein. Such as from 0.1 to 2 or, in particular, 0.5.

In another embodiment, the method of the invention is a method of designing an mRNA which is an O-mRNA suitable for translation by an O-ribosome in a cell also comprising a 2^{nd}-ribosome, wherein the mRNA comprises a 5′ UTR and an ORF, the method comprising:
(a) predicting the free energy difference between the free-folded state of the mRNA and the O-ribosome-bound initiation-competent state of the mRNA (ΔGₜₒₜ(O-ribo)), predicting the free energy difference between the free-folded state of the mRNA and the 2^{nd}-ribosome-bound initiation-competent state of the mRNA (ΔGₜₒₜ(2^{nd}-ribo)), and calculating ΔGₜₒₜ(opt) according to the formula: ΔGₜₒₜ(opt)=ΔGₜₒₜ(O-ribo)−X*ΔGₜₒₜ(2^{nd}-ribo), wherein the 5′ UTR comprises an O-SD;
(b) introducing a modification which is a single nucleotide change, insertion, or deletion into the 5′ UTR, wherein the modification is not introduced into the O-SD five-nucleotide core, or the exchange of any one of codons 2 to 12 within the ORF with a synonymous codon;
(c) predicting the new ΔGₜₒₜ(O-ribo) (ΔGₜₒₜ^{new}(O-ribo)) and the new ΔGₜₒₜ(2^{nd}-ribo) (ΔGₜₒₜ^{new}(2^{nd}-ribo) after modification, and calculating ΔGₜₒₜ^{new}(opt) according to the formula: ΔGₜₒₜ^{new}(opt)=ΔGₜₒₜ^{new}(O-ribo)−X*ΔGₜₒₜ^{new}(2^{nd}-ribo);
(d) accepting the modification if said ΔGₜₒₜ^{new}(opt) is more negative than the preceding ΔGₜₒₜ(opt), and
accepting or rejecting the modification according to a probability distribution if said ΔGₜₒₜ^{new}(opt) is more positive than the preceding ΔGₜₒₜ(opt); wherein the magnitude of the difference between said ΔGₜₒₜ^{new}(opt) and said ΔGₜₒₜ(opt) determines the probability of acceptance, wherein a smaller magnitude is associated with a higher chance of acceptance compared to a larger magnitude; and
(e) iterating steps (b) to (d) until at least 10, 50, 100, 250, or, in particular, 500 consecutive iterations do not lead to a more negative ΔGₜₒₜ^{new}(opt), and then
generating an O-mRNA sequence comprising the 5′ UTR and the ORF which comprise the accepted modification(s). Optionally, wherein X is a number as disclosed herein. Such as from 0.1 to 2 or, in particular, 0.5.

In yet another embodiment, there is provided a method of designing an mRNA which is an O-mRNA suitable for translation by an O-ribosome in a cell also comprising a 2^{nd}-ribosome, wherein the mRNA comprises a 5′ UTR and an ORF, the method comprising:
(a) predicting the free energy difference between the free-folded state of the mRNA and the O-ribosome-bound initiation-competent state of the mRNA (ΔGₜₒₜ(O-ribo)), predicting the free energy difference between the free-folded state of the mRNA and the 2^{nd}-ribosome-bound initiation-competent state of the mRNA (ΔGₜₒₜ(2^{nd}-ribo)), and calculating ΔGₜₒₜ(opt) according to the formula: ΔGₜₒₜ(opt)=ΔGₜₒₜ(O-ribo)−X*ΔGₜₒₜ(2^{nd}-ribo), wherein the 5′ UTR comprises an O-SD;
(b) introducing a modification which is a single nucleotide change, insertion, or deletion into the 5′ UTR, wherein the modification is not introduced into the O-SD five-nucleotide core, or the exchange of any one of codons 2 to 20, 2 to 15, 2 to 12, 2 to 10, or 2 to 5 within the ORF with a synonymous codon;
(c) predicting the new ΔGₜₒₜ(O-ribo) (ΔGₜₒₜ^{new}(O-ribo)) and the new ΔGₜₒₜ(2^{nd}-ribo) (ΔGₜₒₜ^{new}(2^{nd}-ribo) after modification, and calculating ΔGₜₒₜ^{new}(opt) according to the formula: ΔGₜₒₜ^{new}(opt)=ΔGₜₒₜ^{new}(O-ribo)−X*ΔGₜₒₜ^{new}(2^{nd}d-ribo);
(d) accepting the modification if said ΔGₜₒₜ^{new}(opt) is more negative than the preceding ΔGₜₒₜ(opt), and
accepting or rejecting the modification according to

$$exp ( \frac{Δ {G}_{tot}^{new} ( opt ) - Δ {G}_{tot} ( opt )}{{T}_{SA}} )$$

if said ΔGₜₒₜ^{new}(opt) is more positive than the preceding ΔGₜₒₜ(opt);
(e) iterating steps (b) to (d) until at least 10, 50, 100, 250, or, in particular, 500 consecutive iterations do not lead to a more negative ΔGₜₒₜ^{new}(opt), and then
generating an O-mRNA sequence comprising the 5′ UTR and the ORF which comprise the accepted modification(s). Optionally, wherein X is a number as disclosed herein. Such as from 0.1 to 2 or, in particular, 0.5.

Any of the methods of designing an O-mRNA may be used to optimise an O-mRNA to be translated by the O-ribosome at an enhanced rate and/or optimise an O-mRNA to be more orthogonal. Optimised orthogonality may be such that the difference is increased between the translation efficiency of the O-mRNA by an O-ribosome and the translation efficiency of the O-mRNA by a 2^{nd}-ribosome (e.g. a WT-ribosome or a second O-ribosome). This may be calculated by measuring the yield of a protein produced from the O-mRNA in the presence of O-ribosomes, and dividing it by the yield of the protein produced from the O-mRNA in the presence of the 2^{nd}-ribosomes.

The yield obtained when the O-mRNA is in the presence of O-ribosomes may be increased at least 2-fold, 5-fold, 10-fold, 15-fold, 20-fold, 25-fold, 30-fold, 35-fold, or 40-fold, compared to production from an unoptimized sequence.

The orthogonality of the O-mRNA may be increased at least 2-fold, 5-fold, 10-fold, 15-fold, 20-fold, 25-fold, 30-fold, 35-fold, 40-fold, 45-fold, or 50-fold compared to the orthogonality of an unoptimized sequence.

Any of the methods of designing an O-mRNA may further comprise the step of producing a nucleic acid molecule encoding said O-mRNA. The nucleic acid may be a DNA sequence and may be included in a vector suitable for delivery to the intended host cell. As such, a host cell comprising a nucleic acid molecule encoding said O-mRNA is also provided.

Any of the methods of designing an O-mRNA may further comprise the step of experimentally verifying the O-mRNA. In such embodiments, the yield of the encoded protein from the O-mRNA may be compared to the yield of the protein from the unoptimized mRNA sequence or to the yield of the protein when encoded by a WT-mRNA and translated by a WT-ribosome. In addition, or alternatively, the experimental verification may comprise measuring the orthogonality of the O-mRNA, as discussed herein, and optionally comparing it to the orthogonality of the unoptimized mRNA.

The methods of designing an O-mRNA may be performed on a computer. Thus, systems comprising a processor; and one or more computer-readable storage media having stored thereon instructions for execution on said processor to perform the method of the invention are provided. In addition, a computer program product comprising a non-transitory machine readable medium storing program code that, when executed by one or more processors of a computer system, causes the computer system to implement the method of the invention are also provided.

For example, in an embodiment, there is provided a computer-implemented method of designing an mRNA which is an O-mRNA suitable for translation by an O-ribosome, wherein the mRNA comprises a 5′ UTR and an ORF, the method comprising executing program code on one or more processors to implement the following steps:
(a) predicting the free energy difference between the free-folded state of the mRNA and the O-ribosome-bound initiation-competent state of the mRNA (ΔGₜₒₜ(O-ribo));
(b) introducing a modification into the 5′ UTR;
(c) predicting the new ΔGₜₒₜ(O-ribo) (ΔGₜₒₜ^{new}(O-ribo)) after modification;
(d) accepting the modification if said ΔGₜₒₜ^{new}(O-ribo) is more negative than the preceding ΔGₜₒₜ(O-ribo), and
accepting or rejecting the modification according to a probability distribution if said ΔGₜₒₜ^{new}(O-ribo) is more positive than the preceding ΔGₜₒₜ(O-ribo); and
(e) generating an O-mRNA sequence comprising the 5′ UTR which comprises the accepted modification(s). This method may comprise any of the other features or limitations disclosed herein.

In addition to the above, the inventors further provide surprisingly effective methods of designing operons comprising at least two exogenous tRNAs. The inventors have automated the creation of operons for the compact, scalable expression of distinct tRNAs, which may be orthogonal tRNAs. As an example, the inventors develop compact operons expressing engineered triply orthogonal PylRS/tRNA^{Pyl} pairs and an Archaeoglobus fulgidus tyrosyl-tRNA synthetase (AfTyrRS)/tRNA^{Tyr} derived pair, and demonstrate that the operons are highly effective.

Thus, in an aspect of the invention, there is provided a method of designing an operon encoding at least two exogenous tRNAs for expression in a host cell comprising an endogenous genome encoding endogenous tRNAs, the method comprising:
(i) generating permutations of arrangements of the at least two exogenous tRNAs;
(ii) identifying, within the endogenous genome, adjacent pairs of endogenous tRNAs with the highest level of sequence identity to each adjacent pair of exogenous tRNAs within each permutation of the at least two exogenous tRNAs;
(iii) identifying the intergenic region in the endogenous genome between each of the identified adjacent pairs of endogenous tRNAs;
(iv) generating a plurality of sequences encoding each permutation of the at least two exogenous tRNAs and comprising the identified intergenic region(s) positioned between each associated adjacent pair of the exogenous tRNAs; and
(v) selecting a sequence from said plurality of sequences for inclusion in the operon encoding the at least two exogenous tRNAs.

The method may be for designing an operon encoding at least three, four, five, six or more exogenous tRNAs. However, the number of exogenous tRNAs does not have a particular upper limit for the method of the invention to be applicable.

The resultant operon may comprise a first and a second exogenous tRNA, and thus step (i) may comprise generating the arrangements: a) first and then second tRNA and b) second and then first tRNA. Other embodiments may comprise a first, second, and a third exogenous tRNA, and thus step (i) may comprise generating the arrangements: a) first, then second, then third tRNA, b) first, then third, then second tRNA, c) second, then first, then third tRNA, etc. In some embodiments, all possible permutations are generated.

For each of the above-mentioned permutations, the method then comprises associating each pair of exogenous tRNA within the permutation with a pair of endogenous tRNAs within the endogenous genome of the host cell for which the operon is intended. The association is made based on identifying adjacent tRNA pairs within the endogenous genome with the highest level of sequence identity to the adjacent exogenous tRNA pairs. For instance, if the permutation is “first, then third, then second tRNA”, the endogenous adjacent tRNA pairs with the highest level of sequence identity to the first and the third tRNA will be identified, and the endogenous adjacent tRNA pairs with the highest level of sequence identity to the third and the second tRNA will be identified.

The sequence identity may be determined by comparing the acceptor stem sequences of the endogenous tRNAs to the acceptor stem sequences of the exogenous tRNAs. In particular, the first seven and last eight nucleotides, not including the CCA end, of the tRNAs may be compared.

When the intergenic region between the endogenous pairs of tRNAs is identified, the method may optionally set limits on the minimum and/or maximum intergenic regions to be considered. For instance, the minimum intergenic region to be considered may be 5, 10, 15, 20, or 25 base pairs. In a particular embodiment, the minimum intergenic region to be considered is 10 base pairs. The maximum intergenic region to be considered may be 50, 75, 100, 125, or 150 base pairs. In a particular embodiment, the maximum intergenic region to be considered is 100 base pairs. In one embodiment, the minimum intergenic region to be considered is 10 base pairs and the maximum is 100 base pairs.

A plurality of sequences may then be generated encoding the permutations of exogenous tRNAs and the intergenic sequences. For instance, one of the sequences could encode the previous example “first, then third, then second tRNA” and between the first and third tRNA would be the intergenic sequence associated with the pair of endogenous tRNAs most similar to the first and third tRNAs, and between the third and second tRNA would be the intergenic sequence associated with the pair of endogenous tRNAs most similar to the third and second tRNAs.

A sequence may then be selected from the plurality of sequences for inclusion in the operon encoding the exogenous tRNAs. In some embodiments, the plurality of sequences are ranked based on the sum of the sequence identity between the at least two exogenous tRNAs and the corresponding endogenous tRNAs used to define the intergenic regions. The selection may then be made from the ranked list, for instance, the most highly identical sequence may be selected.

Except where a step of the method of designing a tRNA operon is performed on the output of a preceding step, the order of steps is not limited. For instance, adjacent pairs of endogenous tRNAs and the intergenic regions within the endogenous genome may be identified before the method of the invention is begun or during said method. A list of adjacent pairs of endogenous tRNAs and the intergenic regions within the endogenous genome may be pre-prepared before step (i) of the method of the invention.

The methods of designing a tRNA operon result in an operon comprising at least a first sequence encoding a first tRNA and a second sequence encoding a second tRNA, and an intergenic sequence derived from the intended host cell.

In some embodiments, the operon may comprise other ORFs. The tRNAs may be used to interspace other ORFs such that multiple mRNAs may be generated from one promoter. In such embodiments, the methods of designing a tRNA operon may be used to optimize the flanking regions of these tRNAs.

Any of the methods of designing a tRNA operon may further comprise the step of producing a nucleic acid molecule encoding said tRNA operon. The nucleic acid may be a DNA sequence and may be included in a vector suitable for delivery to the intended host cell. As such, a host cell comprising a nucleic acid molecule encoding said tRNA operon is also provided.

Any of the methods of designing a tRNA operon may further comprise the step of experimentally verifying the tRNA operon. In such embodiments, the yield of the encoded tRNAs may be measured when the operon is inserted into a suitable host cell.

In an aspect of the invention, there is provided a host cell comprising an endogenous genome, wherein the host cell comprises a nucleic acid encoding an operon comprising at least two exogenous tRNAs, and wherein the nucleic acid sequence between each pair of exogenous tRNAs is an intergenic sequence derived from the endogenous genome. The operon may be obtained by or obtainable by the methods of designing a tRNA operon of the invention. Thus, the intergenic sequence(s) is the intergenic sequence from between the pairs of endogenous tRNAs with the most identity to the exogenous tRNAs. The host cell may also comprise the endogenous tRNAs from which the intergenic sequences were derived. In other embodiments, one or more endogenous tRNAs are deleted from the host cell.

The methods of designing a tRNA operon may be performed on a computer. Thus, systems comprising a processor; and one or more computer-readable storage media having stored thereon instructions for execution on said processor to perform the method of the invention are provided. The selection step may be performed manually or may be automated. In addition, a computer program product comprising a non-transitory machine readable medium storing program code that, when executed by one or more processors of a computer system, causes the computer system to implement the method of the invention are also provided.

Thus, there is provided a computer-implemented method of designing an operon encoding at least two exogenous tRNAs for expression in a host cell comprising an endogenous genome encoding endogenous tRNAs, the method comprising executing program code on one or more processors to implement the following steps:
(i) generating permutations of arrangements of the at least two exogenous tRNAs;
(ii) identifying, within the endogenous genome, adjacent pairs of endogenous tRNAs with the highest level of sequence identity to each adjacent pair of exogenous tRNAs within each permutation of the at least two exogenous tRNAs;
(iii) identifying the intergenic region in the endogenous genome between each of the identified adjacent pairs of endogenous tRNAs; and
(iv) generating a plurality of sequences encoding each permutation of the at least two exogenous tRNAs and comprising the identified intergenic region(s) positioned between each associated adjacent pair of the exogenous tRNAs; and optionally
(v) selecting a sequence from said plurality of sequences for inclusion in the operon encoding the at least two exogenous tRNAs. This method may comprise any of the other features or limitations disclosed herein.

The inventors further provide surprisingly effective methods of designing polycistronic operons encoding at least two exogenous genes for expression in a host cell. The inventors provide experimental data herein which demonstrate that the methods described herein can be used to achieve high expression of the four exogenous aaRSs in a host cell.

Thus, in an aspect of the invention, there is provided a method of designing an operon comprising at least two exogenous ORFs for expression in a host cell, wherein the method comprises:
(i) generating a plurality of 5′ UTR sequences for each of the at least two exogenous ORFs, wherein each 5′ UTR sequence is optimised for a negative predicted free energy difference between the free-folded state of an mRNA comprising said 5′ UTR sequence and the exogenous ORF and the ribosome-bound initiation-competent state of said mRNA (ΔGₜₒₜ(ribo));
(ii) predicting the ΔGₜₒₜ(ribo) for each of the 5′ UTR sequences when positioned 5′ to the exogenous ORF for which said 5′ UTR was optimised and positioned 3′ to each one of the remaining at least two exogenous ORFs; and
(iii) selecting an arrangement of the 5′ UTR sequences and the at least two exogenous ORFs.

Step (i) may comprise generating two, three, four, five, or more 5′ UTR sequences for each of the at least two exogenous ORFs. In some examples, six, seven, eight, nine, ten, 15, 20 or more 5′ UTR sequences are generated. In a particular embodiment, five 5′ UTR sequences are generated for each exogenous ORF. For instance, if the operon includes three exogenous ORFs, then fifteen 5′ UTR sequences may be generated, a set of five for each exogenous ORF.

Each 5′ UTR sequence is optimised for a negative predicted free energy difference between the free-folded state of an mRNA comprising said 5′ UTR sequence and the exogenous ORF and the ribosome-bound initiation-competent state of said mRNA (ΔGₜₒₜ(ribo)). As such, each 5′ UTR is optimised for efficient translation by a ribosome.

A method for the predication of ΔGₜₒₜ(ribo) is described in detail in the Examples section. In an embodiment, ΔGₜₒₜ(ribo) is the sum of the free energy required to unfold the mRNA (ΔG_{unfolding}) and the free energy released upon the mRNA binding to a ribosome to form a ribosome-bound initiation-competent state (ΔG_{ribo binding}).

In an embodiment, the ΔGₜₒₜ(ribo) is predicted according to the following:

ΔGₜₒₜ(ribo)=(ΔG_{mRNA-rRNA}+ΔGₛₜₐᵣₜ+ΔG_{spacing}−ΔG_{standby})+ΔG_{unfolding}; wherein
ΔG_{mRNA-rRNA} is the free energy of a predicted co-folded secondary structure of the last 9 nucleotides of a 16S rRNA and the mRNA;
ΔGₛₜₐᵣₜ is the energy released from binding of an initiator tRNA to the start codon of the sequence encoding the exogenous ORF;
ΔG_{spacing} is an energy penalty for non-optimal spacing length between the Shine Dalgarno sequence and the start codon of the sequence encoding the exogenous ORF;
ΔG_{standby} is the energy required to unfold secondary structures that sequester the four nucleotides upstream of the Shine Dalgarno sequence; and
ΔG_{unfolding} is the energy required to unfold secondary structures in the mRNA.

Further information is provided in relation to O-mRNA optimisation.

The method of optimising the 5′ UTR for efficient translation by a ribosome may comprise:
(a) introducing a modification into the 5′ UTR;
(b) predicting the new ΔGₜₒₜ(ribo) (ΔGₜₒₜ^{new}(ribo)) after modification;
(c) accepting the modification if said ΔGₜₒₜ^{new}(ribo) is more negative than the preceding ΔGₜₒₜ(ribo), and
accepting or rejecting the modification according to a probability distribution if said ΔGₜₒₜ^{new}(ribo) is more positive than the preceding ΔGₜₒₜ(ribo); and
(d) generating a 5′ UTR sequence comprising the accepted modification(s).

The method may be as described in relation to O-mRNA optimisation.

During the method of the invention, the modification is accepted if said ΔGₜₒₜ^{new}(ribo) is more negative than the preceding ΔGₜₒₜ(ribo). The “preceding” ΔGₜₒₜ(ribo) is the ΔGₜₒₜ(ribo) predicted before the modification is made. As discussed herein, the methods of the invention may be iterated, and so the preceding ΔGₜₒₜ(ribo) may be the ΔGₜₒₜ^{new}(ribo) calculated during the previous iteration.

The acceptance of a modification during the method of the invention means that the sequence alteration introduced by the modification is maintained for the next iteration of the method or, if the there is no further iteration of the method, is maintained in the sequence which is the output of the method.

During the method of the invention, the modification is accepted or rejected according to a probability distribution if said ΔGₜₒₜ^{new}(ribo) is more positive than the preceding ΔGₜₒₜ(ribo). The “preceding ΔGₜₒₜ(ribo)” is as discussed above. The probability distribution may be based upon conditional probability, wherein the chance of acceptance decreases as the difference between ΔGₜₒₜ^{new}(ribo) and ΔGₜₒₜ(ribo) increases. The probability may be a Monte-Carlo optimisation.

In an embodiment, the probability distribution according to which the modification is accepted or rejected is:

$$exp ( \frac{\vert Δ {G}_{tot}^{new} ( ribo ) - Δ {G}_{tot} ( ribo ) \vert}{{T}_{SA}} )$$

wherein T_{SA} is the simulated annealing temperature.

The T_{SA} may be adjusted in any manner as disclosed herein. In a particular embodiment, the T_{SA} is adjusted to maintain a 5-20% acceptance rate.

The rejection of a modification during the method of the invention means that the sequence alteration introduced by the modification is reversed and so not maintained for the next iteration of the method, or not maintained in the output sequence.

In an embodiment, the modification is or comprises a single nucleotide change, insertion, or deletion. In another embodiment, the modification is either introduced into the 5′ UTR or is the exchange of any one of codons 2 to 20, 2 to 15, 2 to 12, 2 to 10, or 2 to 5 with a synonymous codon within the sequence encoding the exogenous ORF. In a particular embodiment, the modification comprises a single nucleotide change, insertion, or deletion into the 5′ UTR, or the exchange of any one of codons 2 to 12 within the ORF with a synonymous codon.

The method of designing an operon comprising at least two exogenous ORFs may be iterated such that a plurality of modifications are considered for acceptance or rejection and the final output sequence includes the cumulative effect of all of said accepted modifications. The iteration may be any as disclosed herein. In particular, steps (a) to (c) of the method of the invention may be iterated. In an embodiment, the method is iterated at least 200, 300, 400, 500, 1000, 5000, or, in particular, 10000 times. In other embodiments, the method may be iterated until consecutive iterations do not lead to a more negative ΔGₜₒₜ^{new}(ribo), as disclosed herein. For instance, the steps (a) to (c) may be iterated until at least 10, 50, 100, 250, 500, 1000, 2000, 3000, 5000, or 10000 consecutive iterations consecutive iterations do not lead to a more negative ΔGₜₒₜ^{new}(ribo).

The initial 5′ UTR considered for optimisation may have the lengths and properties as described in relation to O-mRNA optimisation. In particular, he initial 5′ UTR may be from 30 to 40 nucleotides long, or in particular is 35 nucleotides. Alternatively, the 5′ UTR may be longer but a 30-40, or in particular 35, nucleotide window is considered by the methods of the invention for modification. The 35-nucleotide window may be the 35 nucleotides of the 5′ UTR that are closest to the start codon. In other embodiments, the initial 5′ UTR may be shorter, such as a 15, 20, or 25 nucleotide 5′ UTR, or longer, such as at least 40, 50, or more nucleotides. It might be desirable to generate a 5′ UTR which is of a particular length, in which case a 15, 20, 25, 30, 35, 45, 50 nucleotide window may be considered such that a particular length of output sequence may be achieved. The 5′ UTR to which step (a) is applied may comprise a wild type Shine Dalgarno sequence, or the five-nucleotide core of a wild type Shine Dalgarno sequence. The 5′ UTR may be of a random sequence apart from the Shine Dalgarno sequence. The Shine Dalgarno sequence may be five nucleotides from the start codon of the ORF, which is predicted to be the optimal spacing.

The method of designing an operon comprising at least two exogenous ORFs is not limited to a specific number of exogenous ORFs. For instance, the method may be used to design an operon comprising at least three, at least four, at least five, or at least six exogenous ORFs.

The method of designing an operon comprising at least two exogenous ORFs is not limited to use with particular types of exogenous ORF. The experimental data provided herein provide proof of principle for operons comprising multiple sequences encoding aaRSs. As such, in an embodiment, at least one of the exogenous ORFs encodes an aaRS. In another embodiment, the method may be for designing a polycistronic operon encoding at least two, three, four, five, or six aaRSs.

Step (ii) of the method of designing a polycistronic operon comprises predicting the ΔGₜₒₜ(ribo) for each of 5′ UTR sequences when positioned 5′ to the exogenous ORF for which said 5′ UTR was optimised and positioned 3′ to each one of the remaining at least two exogenous ORFs (see FIG. 7, supplementary FIG. 3). As such, a 5′ UTR, which is optimised for translation of one of the exogenous ORFs, is then considered in the context of being positioned 3′ of one of the other exogenous ORFs and the translational efficiency is again measured. This is performed for each of the other exogenous ORFs. For instance, in an embodiment where the operon has three exogenous ORFs, a particular 5′ UTR optimised for the first exogenous ORF is considered when positioned 3′ of the second exogenous ORF and separately when positioned 3′ of the third exogenous ORF.

Step (iii) of the method of designing a polycistronic operon comprises the selection of an arrangement of the 5′ UTR sequences and the at least two exogenous ORFs. The selected arrangement may be chosen such that each exogenous ORF is predicted to be translated at a high level. For instance, the ΔGₜₒₜ(ribo) for each 5′ UTR/exogenous ORF pair within the operon may be predicted and added together, and the arrangement with the most negative cumulative ΔGₜₒₜ(ribo) may be chosen. In other embodiments, an arrangement with the most negative average ΔGₜₒₜ(ribo) for all 5′ UTR/exogenous ORF pairs within the operon may be chosen. The average may be the mean. In addition, an arrangement wherein each 5′ UTR/exogenous ORF pair has a ΔGₜₒₜ(ribo) which is more negative than a target ΔGₜₒₜ(ribo) may be chosen. The target may be chosen to ensure a particular yield of the product of each exogenous ORF within a host cell. For instance, the target may be of a level that would ensure that the exogenous ORF is translated at a level sufficient for the protein product to achieve its function. For example, if the exogenous ORF encodes an aaRS, the target ΔGₜₒₜ(ribo) may be such that adequate aaRS protein would be produced in a desired host cell to ensure that the aaRS would function with its cognate tRNA during protein synthesis.

In a particular embodiment, step (iii) comprises the selection of an arrangement with the most negative average ΔGₜₒₜ(ribo) for all 5′ UTR/exogenous ORF pairs within the operon, and wherein each 5′ UTR/exogenous ORF pair has a ΔGₜₒₜ(ribo) which is more negative than a target ΔGₜₒₜ(ribo).

Any of the methods of designing an operon comprising exogenous ORFs may further comprise the step of producing a nucleic acid molecule encoding said operon. The nucleic acid may be a DNA sequence and may be included in a vector suitable for delivery to the intended host cell.

Any of the methods of designing an operon encoding exogenous ORFs may further comprise the step of experimentally verifying the operon. In such embodiments, the yield of the encoded proteins may be measured when the operon is inserted into a suitable host cell. The experimental verification may form part of selecting an arrangement of the 5′ UTR sequences and the at least two exogenous ORFs.

In an aspect of the invention, there is provided a host cell comprising a nucleic acid encoding an operon comprising at least two exogenous ORFs, wherein the operon is obtained by or obtainable by the methods of designing an operon disclosed herein.

The method of designing a polycistronic operon comprising at least two exogenous ORFs may be implemented on a computer. In some embodiments, step (iii) may be performed manually. Thus, systems comprising a processor; and one or more computer-readable storage media having stored thereon instructions for execution on said processor to perform the method of the invention are provided. In addition, a computer program product comprising a non-transitory machine readable medium storing program code that, when executed by one or more processors of a computer system, causes the computer system to implement the method of the invention are also provided.

Thus, there is provided a computer-implemented method of designing an operon comprising at least two exogenous ORFs for expression in a host cell, the method comprising executing program code on one or more processors to implement the following steps:
(i) generating a plurality of 5′ UTR sequences for each of the at least two exogenous ORFs, wherein each 5′ UTR sequence is optimised for a negative predicted free energy difference between the free-folded state of an mRNA comprising said 5′ UTR sequence and the exogenous ORF and the ribosome-bound initiation-competent state of said mRNA (ΔGₜₒₜ(ribo));
(ii) predicting the ΔGₜₒₜ(ribo) for each of the 5′ UTR sequences when positioned 5′ to the exogenous ORF for which said 5′ UTR was optimised and positioned 3′ to each one of the remaining at least two exogenous ORFs; and optionally
(iii) selecting an arrangement of the 5′ UTR sequences and the at least two exogenous ORFs. This method may comprise any of the other features or limitations disclosed herein.

The inventors have successfully combined all of the above advances to create a 68-codon, 24 amino acid genetic code and to efficiently incorporate four distinct ncAAs in response to four distinct orthogonal codons, via O-ribosome-mediated translation of an O-mRNA. As discussed in the Examples section, the inventors use this system to generate, for the first time, a protein comprising 20 canonical amino acids and four non-canonical amino acids.

Thus, in an aspect of the invention, there is provided a host cell comprising:
a nucleic acid sequence encoding an O-mRNA which encodes an exogenous protein, wherein the O-mRNA is obtained or is obtainable by any method of designing an O-mRNA of the invention, and wherein the O-mRNA comprises at least two types of orthogonal codon;
a nucleic acid sequence comprising an O-tRNA operon encoding at least two orthogonal tRNAs, wherein the at least two orthogonal tRNAs are capable of decoding said at least two types of orthogonal codon, wherein the operon is obtained or is obtainable by any method of designing a tRNA operon of the invention;
a nucleic acid sequence comprising an orthogonal aminoacyl-tRNA synthetase (O-aaRS) operon encoding at least two O-aaRSs, wherein the at least two O-aaRSs form O-aaRS-O-tRNA pairs with the at least two orthogonal tRNAs, wherein the operon is obtained or is obtainable by any method of designing a tRNA operon of the invention; and an orthogonal ribosome.

In an embodiment, the O-tRNA and O-aaRS operons are present within the same nucleic acid sequence. For instance, these two operons may have been introduced into the host cell via a single vector.

The exogenous protein encoded by the O-mRNA may be any protein for which production is desired. For instance, the exogenous protein may be a therapeutic protein, such as an antibody or a cytokine.

The host cells comprise at least two O-aaRSs and at least two O-tRNAs. These function in pairs, i.e. they form a first aaRS/tRNA pair and a second aaRS/tRNA pair. One pair is capable of decoding one of the types of orthogonal codon and the other pair is capable of decoding the other type of orthogonal codon. Both pairs are capable of functioning with the O-ribosome.

In other embodiments the host cells of the invention comprise at least a third and optionally at least a fourth O-aaRS-O-tRNA pair. In such embodiments, the O-mRNA may comprise at least a third and optionally at least a fourth type of orthogonal codon. The third aaRS-tRNA pair is capable decoding the third type of orthogonal codon and the fourth aaRS-tRNA pair is capable of decoding the fourth type of orthogonal codon. Further sets of O-aaRS, O-tRNA, and orthogonal codon may be included. All orthogonal components are capable of functioning with the O-ribosome.

The O-aaRSs do not recognize endogenous tRNAs, and specifically aminoacylate an orthogonal cognate tRNA (which is not an efficient substrate for endogenous synthetases) with non-canonical amino acids provided to (or synthesised by) the cell (Chin, J. W., 2017. Nature, 550(7674), 53-60).

The O-ribosome may be any disclosed herein. In particular, the O-ribosome may be O-riboQ1, any O-ribosome disclosed in or obtainable by a method disclosed in WO2008/065398A1, any O-ribosome disclosed in or obtainable by a method disclosed in WO2011/077075A1, or any O-ribosome disclosed in or obtainable by a method disclosed in any of Neumann, H et al. Nature 464, 441-444 (2010); Wang, K. et al. Nat. Biotechnol. 25, 770-777 (2007); or Schmied, W. H. et al. Nature 564, 444-448 (2018).

The aminoacyl-tRNA synthetases used herein may be varied. Although specific tRNA synthetase sequences may have been used in the examples, the invention is not intended to be confined only to those examples. In principle any aminoacyl-tRNA synthetase which provides a tRNA charging (aminoacylation) function and functions with an O-ribosome can be employed. For example, the tRNA synthetase may be from any suitable species such as from archaea, for example from Methanosarcina—such as Alethanosarcina barkeri MS; Methanosarcina barkeri str. Fusaro; Methanosarcina mazei G01; Methanosarcina acetivorans C2A; Methanosarcina thermophila; or Methanococcoides—such as Methanococcoides burtonii. Alternatively the tRNA synthetase may be from bacteria, for example from Desulfitobacterium—such as Desulfitobacterium hafniense DCB-2; Desulfitobacterium hafniense Y51; Desulfitobacterium hafniense PCP1; or Desulfotomaculum acetoxidans DSM 771.

The aminoacyl-tRNA synthetase may be a pyrrolysyl tRNA synthetase (PylRS). The PylRS may be a wild-type or a genetically engineered PylRS. Genetically engineered PylRS has been described, for example, by Neumann et al. (Nat Chem Biol 4:232, 2008) and by Yanagisawa et al. (Chem Biol 2008, 15:1187), in EP2192185A1, and in WO2016/066995 (each incorporated herein by reference). Suitably, a genetically engineered tRNA synthetase gene is selected that increases the incorporation efficiency of non-canonical amino acid(s). The PylRS may be Methanosarcina barkeri (MbPylRS) or Methanosarcina mazei (MmPylRS).

The tRNA used herein may be varied. Although specific tRNAs may have been used in the examples, the invention is not intended to be confined only to those examples. In principle, any tRNA can be used provided that it is compatible with the selected tRNA synthetase and the O-ribosome.

The tRNA may be from any suitable species such as from archea, for example from Methanosarcina—such as Methanosarcina barkeri MS; Methanosarcina barkeri str. Fusaro; Methanosarcina mazei. G01; Methanosarcina acetivorans C2A; Methanosarcina thermophila; or Methanococcoides—such as Methanococcoides burtonii. Alternatively the tRNA may be from bacteria, for example from Desulfitobacterium—such as Desulfitobacterium hafniense DCB-2; Desulfitobacterium hafniense Y51; Desulfitobacterium hafniense PCP1; or Desulfotomaculum acetoxidans DSM 771.

The tRNA gene can be a wild type tRNA gene or it may be a mutated tRNA gene. Suitably, a mutated tRNA gene is selected that increases the incorporation efficiency of unnatural amino acid(s). In one embodiment, the mutated tRNA gene is a U25C variant of PylT as described in Biochemistry (2013) 52, 10 (incorporated herein by reference).

In one embodiment, the mutated tRNA gene is an Opt variant of PylT as described in Fan et al. (Nucleic Acids Research doi:10.1093/nar/gkv800) (incorporated herein by reference herein).

In one embodiment, the mutated tRNA gene has both the U25C and the Opt variants of PylT, i.e. in this embodiment the tRNA, such as the PylT tRNA_{CUA} gene, comprises both the U25C and the Opt mutations.

In one embodiment, the sequence encoding the tRNA is the pyrrolysine tRNA (PylT) gene from Methanosarcina mazei pyrrolysine which encodes tRNAPyl.

The aminoacyl-tRNA synthetase and tRNA pair may be as disclosed in, or adapted from those disclosed in, Cervettini et al. (Rapid discovery and evolution of orthogonal aminoacyl-tRNA synthetase-tRNA pairs, Nature Biotechnology, Vol 38, 990 August 2020, P989-999) or Dunkelmann et al. (Engineered triply orthogonal pyrrolysyl-tRNA synthetase/tRNA pairs enable the genetic encoding of three distinct non-canonical amino acids, Nature Chemistry, Vol 12, June 2020 P535-544). Each of these documents is incorporated by reference.

The aaRS, tRNA, and codon sets preferably function together and are orthogonal to each endogenous amino acid, aaRS and group of isoacceptor tRNAs and their cognate group of codons.

At least one of the orthogonal codons may be a quadruplet codon. At least one of the orthogonal codons may be a stop codon, such as an amber codon. At least one of the orthogonal codons may be a reassigned sense codon in a genomically recoded prokaryotic cell (see: WO2020/229592; or Robertson et al.; Sense codon reassignment enables viral resistance and encoded polymer synthesis; Science; 2021; Vol. 372, Issue 6546, pp. 1057-1062). In a particular embodiment, all of the orthogonal codons may be quadruplet codons. In a particular embodiment, the O-mRNA comprises a first, second, third, and fourth type of orthogonal codon, each of which is a quadruplet codon.

The host cell may be a prokaryotic cell. The host cell may be a bacterial cell, such as E. coli. The host cell may be capable of producing a protein comprising all twenty canonical amino acids and at least four non-canonical amino acids.

The substrate of the orthogonal tRNA synthetases may be any non-canonical amino acid. Hence, the cell of the invention may be used to generate polypeptides comprising at least a first non-canonical amino acid, at least a second non-canonical amino acid, at least a third non-canonical amino acid, and at least a fourth non-canonical amino acid.

Thus, in another aspect of the invention, there is provided a method of producing a polypeptide, comprising:
providing a host cell of the invention;
incubating the host cell in the presence of a first non-canonical amino acid, wherein the first non-canonical amino acid is a substrate for the one of the O-aaRSs; and
incubating the host cell to allow incorporation of the first non-canonical amino acid into the polypeptide via the O-aaRS-O-tRNA pair.

As discussed, the host cells may comprise a first, second, third, and fourth orthogonal aaRS-tRNA pair. The first pair is capable of decoding a first type of codon to incorporate a first non-canonical amino acid, the second pair is capable of decoding a second type of codon to incorporate a second non-canonical amino acid, the third pair is capable of decoding a third type of codon to incorporate a third non-canonical amino acid, and the fourth pair is capable of decoding a fourth type of codon to incorporate a fourth non-canonical amino acid.

As used herein, the term “non-canonical amino acid” means any amino acid excluding L-alanine, L-cysteine, L-aspartic acid, L-glutamic acid, L-phenylalanine, glycine, L-histidine, L-isoleucine, L-lysine, L-leucine, L-methionine, L-asparagine, L-proline, L-glutamine, L-arginine, L-serine, L-threonine, L-valine, L-tryptophan, and L-tyrosine.

The non-canonical amino acid may be an unnatural amino acid. As used herein, an “unnatural amino acid” is any amino acid that is not naturally encoded or found in the genetic code. Such amino acids may be non-proteinogenic amino acids. Thus, an unnatural amino acid may be any amino acid excluding L-alanine, L-cysteine, L-aspartic acid, L-glutamic acid, L-phenylalanine, glycine, L-histidine, L-isoleucine, L-lysine, L-leucine, L-methionine, L-asparagine, L-proline, L-glutamine, L-arginine, L-serine, L-threonine, L-valine, L-tryptophan and L-tyrosine, L-pyrrolysine, and L-selenocysteine.

The non-canonical amino acids that are suitable for use with the present invention are not particularly limited. Suitable non-canonical amino acids will be well known to those of skill in the art, for example those disclosed in Neumann, H., 2012. FEBS letters, 586(15), pp. 2057-2064; and Liu, C. C. and Schultz, P. G., 2010. Annual review of biochemistry, 79, pp. 413-444 (herein incorporated by reference). In some embodiments the non-canonical amino acids are selected from one or more of: p-Acetylphenylalanine, m-Acetylphenylalanine, O-allyltyrosine, Phenylselenocysteine, selenocysteine, p-Propargyloxyphenylalanine, p-Azidophenylalanine, p-Boronophenylalanine, O-methyltyrosine, p-Aminophenylalanine, p-Cyanophenylalanine, m-Cyanophenylalanine, p-Fluorophenylalanine, p-Iodophenylalanine, p-Bromophenylalanine, p-Nitrophenylalanine, L-DOPA, 3-Aminotyrosine, 3-Iodotyrosine, p-Isopropylphenylalanine, 3-(2-Naphthyl)alanine, Biphenylalanine, Homoglutamine, D-tyrosine, p-Hydroxyphenyllactic acid, 2-Aminocaprylic acid, Bipyridylalanine, HQ-alanine, p-Benzoylphenylalanine, o-Nitrobenzylcysteine, o-Nitrobenzylserine, 4,5-Dimethoxy-2-nitrobenzylserine, o-Nitrobenzyllysine, o-Nitrobenzyltyrosine, 2-Nitrophenylalanine, Dansylalanine, p-Carboxymethylphenylalanine, 3-Nitrotyrosine, Sulfotyrosine, Acetyllysine, Methylhistidine, 2-Aminononanoic acid, 2-Aminodecanoic acid, Pyrrolysine, Cbz-lysine, Boc-lysine, Allyloxycarbonyllysine, N^{ε}-((tert-butoxy)carbonyl)-L-lysine (BocK), Nε-(carbobenzyloxy)-L-lysine (CbzK), N^{ε}-allyloxycarbonyl-L-lysine (AllocK), (S)-2-Amino-3-(4-iodophenyl)propanoic acid (p-I-Phe), CypK, AlkK, 3-Nitro-Tyr, and p-Az-Phe. The first, second, and third non-canonical amino acid may be any combination of the aforementioned non-canonical amino acids.

In particular embodiments, the non-canonical amino acids may be any combination of BocK, CbzK, AllocK, p-I-Phe, CypK, AlkK, 3-Nitro-Tyr, and p-Az-Phe.

The host cells of the invention can be used to generate products that are not obtainable by any other methods. As such, in an aspect of the invention, there is provided a polypeptide or a protein containing at least four genetically incorporated non-canonical amino acids, which is obtained or obtainable by the methods disclosed herein.

Sequence comparisons can be conducted with the aid of readily available sequence comparison programs. These publicly and commercially available computer programs can calculate sequence identity between two or more sequences.

The skilled technician will appreciate how to calculate the percentage identity between two nucleic sequences. In order to calculate the percentage identity between two nucleic sequences, an alignment of the two sequences must first be prepared, followed by calculation of the sequence identity value. The percentage identity for two sequences may take different values depending on: (i) the method used to align the sequences, for example, the Needleman-Wunsch algorithm (e.g. as applied by Needle(EMBOSS) or Stretcher(EMBOSS), the Smith-Waterman algorithm (e.g. as applied by Water(EMBOSS)), or the LALIGN application (e.g. as applied by Matcher(EMBOSS); and (ii) the parameters used by the alignment method, for example, local versus global alignment, the matrix used, and the parameters applied to gaps.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (iv) the number of equivalenced positions excluding overhangs. Furthermore, it will be appreciated that percentage identity is also strongly length-dependent. Therefore, the shorter a pair of sequences is, the higher the sequence identity one may expect to occur by chance.

A calculation of percentage identities between two nucleic acid sequences may then be calculated from such an alignment as (N/T)*100, where N is the number of positions at which the sequences share an identical residue, and T is the total number of positions compared including gaps but excluding overhangs.

The sequence alignment may be a pairwise sequence alignment. Suitable services include Needle (EMBOSS), Stretcher (EMBOSS), Water (EMBOSS), Matcher (EMBOSS), LALIGN, or GeneWise. In an example, the identity between two amino acid sequences may be calculated using the service Needle(EMBOSS) set to the default parameters, e.g. matrix (BLOSUM62), gap open (10), gap extend (0.5), end gap penalty (false), end gap open (10), and end gap extend (0.5). In another example, the identity between two amino acid sequences may be calculated using the service Matcher (EMBOSS) set to the default parameters, e.g. matrix (BLOSUM62), gap open (14), gap extend (4), alternative matches (1). In an example, the identity between two nucleic acid sequences may be calculated using the service Needle(EMBOSS) set to the default parameters, e.g. matrix (DNAfull), gap open (10), gap extend (0.5), end gap penalty (false), end gap open (10), and end gap extend (0.5). In another example, the identity between two nucleic acid sequences may be calculated using the service Matcher (EMBOSS) set to the default parameters, e.g. matrix (DNAfull), gap open (16), gap extend (4), alternative matches (1).

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made to the Examples, which are not intended to limit the invention in any way.

### EXAMPLES

The inventors demonstrate 68-codon genetic code for the incorporation of four distinct non-canonical amino acids, which is enabled by automated orthogonal mRNA discovery.

Orthogonal (O-) ribosome mediated translation of O-mRNAs enables the incorporation of up to three distinct non-canonical amino acids (ncAAs) into a protein in Escherichia coli. However, the general and efficient incorporation of multiple distinct ncAAs by O-ribosomes requires scalable strategies for both creating efficiently and specifically translated O-mRNAs, and the compact expression of multiple O-aminoacyl-tRNA synthetase (O-aaRS)/O-tRNA pairs. The inventors automate the discovery of O-mRNAs that lead to up to 40-times more protein, and are up to 50-fold more orthogonal, than previous O-mRNAs; protein yields from our O-mRNAs match or exceed those from wild-type mRNAs. These advances enable a 33-fold increase in yield for incorporating three distinct ncAAs. In addition, the inventors automate the creation of operons for O-tRNAs, and develop operons for O-aaRSs. Finally, the inventors combine these advances to create a 68-codon, 24 amino acid genetic code and efficiently incorporate four distinct ncAAs in response to four distinct quadruplet codons.

### Example 1—Automating 5′ UTR Discovery for Efficient Translation by O-Ribosomes

For our ₛₜᵣₑₚGFP_{His6} ORF on a 5′ UTR containing a wt RBS the predicted ΔGₜₒₜ(wt ribo) is −0.5 kcal/mol. In contrast, when we altered the anti-Shine Dalgarno sequence (aSD) used in the thermodynamic model to that of the O-ribosome the calculated free energy change for orthogonal translation (ΔGₜₒₜ (O-ribo)) of O(trans)-_{Strep}GFP_{His6} was +3.5 kcal/mol. We decided to test whether an equilibrium model of initiation combined with a simulated annealing optimization algorithm, developed for wt translation²⁷, could be adapted to design O-mRNA sequences that are more efficiently translated by O-ribosomes than O(trans)-ₛₜᵣₑₚGFP_{His6} (FIG. 1a,b). We therefore varied the 5′ UTR sequence between the +1 transcription site and the ₛₜᵣₑₚGFP_{His6} ORF and searched—through a simulated annealing optimization algorithm²⁷—for sequences with highly favourable ΔGₜₒₜ (O-ribo) for this ORF.

Using this algorithm (vol 1) we identified four new ₛₜᵣₑₚGFP_{His6} constructs with optimised 5′ UTR regions (O1-ₛₜᵣₑₚGFP_{His6} to O4-ₛₜᵣₑₚGFP_{His6}) for the production of ₛₜᵣₑₚGFP_{His6} protein by the O-ribosome. The ΔGₜₒₜ(O-ribo) for these constructs was: O1-ₛₜᵣₑₚGFP_{His6}-5.8 kcal/mol, O2-ₛₜᵣₑₚGFP_{His6}-4.9 kcal/mol, O3-ₛₜᵣₑₚGFP_{His6}-5.1 kcal/mol, O4-ₛₜᵣₑₚGFP_{His6}-6.6 kcal/mol. Thus, we predicted that these constructs may lead to higher protein levels than O(trans)-ₛₜᵣₑₚGFP_{His6}. We produced ₛₜᵣₑₚGFP_{His6} from cells containing each construct and the O-ribosome. The optimised sequences (O1-ₛₜᵣₑₚGFP_{His6} to O4-ₛₜᵣₑₚGFP_{His6}) led to large (11- to 31-fold) increases in protein production with orthogonal translation compared to O(trans)-ₛₜᵣₑₚGFP_{His6} (FIG. 1c and Supplementary FIG. 1). The level of ₛₜᵣₑₚGFP_{His6} protein produced from O1-ₛₜᵣₑₚGFP_{His6} by the O-ribosome was comparable to that from the original construct containing a wt RBS and translated by the wt ribosome. ΔGₜₒₜ (wt-ribo) (FIG. 1a) for the new sequences was greater than +5 kcal/mol in all cases. Thus ΔG_{orthogonality} (FIG. 1a) predicts that these constructs will be selectively translated by the O-ribosome. Consistent with this prediction, additional experiments demonstrated that translation of ₛₜᵣₑₚGFP_{His6} from each new 5′ UTR was O-ribosome dependent, and the orthogonality of the new sequences was 12- to 19-fold 143 greater than that of O(trans)-ₛₜᵣₑₚGFP_{His6} (FIG. 1c and Supplementary FIG. 1a).

### Example 2—Automating 5′ UTR and ORF Discovery for Scalable, Efficient and Selective Orthogonal Translation

In an effort to fully automate the discovery of 5′ UTRs that do not direct efficient translation by wt ribosomes and direct maximal protein production by the O-ribosome, we designed a new automated search (vol 2) (FIG. 1b). Our new search introduced an explicit penalty for 5′ UTR sequences that are predicted to be substrates for wt ribosomes and was biased towards sequences containing an optimally spaced canonical O-RBS sequence.

The vol 2 search started from a 35 nt 5′ UTR which contained a 9 nt orthogonal SD (O-SD) sequence that is predicted to form perfect Watson-Crick base pairs with the orthogonal aSD sequence at the 3′ end of the O-16S rRNA. The spacing between the O-SD sequence and the start codon was set to 5 nucleotides and the sequence of the 5′ UTR, except the O-SD, was randomized. We then searched for sequences that maximize ΔGₜₒₜ(O-ribo) but minimize ΔGₜₒₜ (wt ribo). We disallowed mutations in the 5-nucleotide core of the O-SD site (TGGGA), which is predicted to base pair with the O-16S rRNA—but not the wt 16S rRNA—and thus determines orthogonality. Using the vol 2 algorithm we created new 5′ UTRs for ₛₜᵣₑₚGFP_{His6} (O5- to O8-ₛₜᵣₑₚGFP_{His6}). These sequences had higher mean ΔGₜₒₜ(O-ribo) (−7.7±0.4 kcal/mol) than those derived from vol 1 (−5.6±0.8 kcal/mol) (Supplementary Table 1). These sequences provided up to 18-fold more ₛₜᵣₑₚGFP_{His6} protein than O(trans)-ₛₜᵣₑₚGFP_{His6} (FIG. 11 and Supplementary FIG. 1b). To investigate the generality of the vol 2 algorithm for enhancing protein production we investigated orthogonal translation of two additional ORFs, mCherry and E2Crimson. O(trans)-mCherry, and O(trans)-E2Crimson (in which the O(trans) 5′ UTR was placed between the +1 base of transcription and the ATG start codon) led to low levels of orthogonal translation. Applying the vol 2 algorithm led to mCherry expression constructs that are up to 10 times more active with the O-ribosome than O(trans)-mCherry, and also up to 8-fold more orthogonal (FIG. 1d and Supplementary FIG. 1c). Similarly, applying the vol 2 algorithm led to E2Crimson production constructs that are up to 14-fold more active with the O-ribosome than O(trans)-E2Crimson, and up to 9-fold more orthogonal; E2Crimson was produced by the O-ribosome from O1-E2Crimson (discovered using the vol 2 algorithm) at comparable levels to the levels produced from a wt RBS using a wt ribosome (FIG. 1e and Supplementary FIG. 1d).

The first 35 nucleotides of ORF sequence can contribute substantially to protein yields^{24, 26, 29}. However, it remains controversial to what extent changing codons to their synonyms in this sequence influences translation through effects on mRNA secondary structure versus effects that result from the decoding of different synonyms with distinct isoacceptor tRNAs^{24-26, 34-36}. We realized that varying codons within the first 35 nucleotides of the ORF to synonymous codons would provide additional degrees of freedom in the computational search for mRNAs that maximize ΔGₜₒₜ (O-ribo) but minimize ΔGₜₒₜ (wt ribo). And we hypothesized that, in some cases, this may allow us to discover mRNAs that are more efficiently translated by the O-ribosome and are more orthogonal with respect to translation by wt ribosomes. To investigate this hypothesis, we allowed codons 2 to 12 of each ORF to vary to their synonyms. We thereby created a third algorithm (vol 3), which builds on vol 2, to explore simultaneous variation in the ORF and 5′ UTR (FIG. 1b).

The vol 3 algorithm provided a notable increase in ΔGₜₒₜ (O-ribo) (ₛₜᵣₑₚGFP_{His6}: −12.6±0.2 kcal/mol; mCherry: −13.5±0.3 kcal/mol; E2Crimson: −13.2±0.0 kcal/mol) with respect to vol 2 (ₛₜᵣₑₚGFP_{His6}: −7.7±0.4; mCherry: −9.6±0.5 kcal/mol; E2Crimson: −8.9±0.5 kcal/mol) and maintained the minimized ΔGₜₒₜ(wt ribo) from vol 2. We discovered 0-mRNA sequences for ₛₜᵣₑₚGFP_{His6} and mCherry that are more orthogonal than those from the vol 2 algorithm and produce protein at levels higher than those produced by wt ribosomes from wt messages (FIG. 1c-e and Supplementary FIG. 1b-d). Overall, our vol 2 and vol 3 algorithms provided protein yields that are 41-, 31- and 14-fold (for ₛₜᵣₑₚGFP_{His6}, mCherry, and E2Crimson, respectively) greater than when the O(trans) 5′ UTR was used with each ORF, and these yields match or exceed the yields from wt ribosomes on wt messages. The orthogonality of the best sequences we have discovered is 31-, 49- and 9-fold (for ₛₜᵣₑₚGFP_{His6}, mCherry, and E2Crimson, respectively) higher than when the O(trans) 5′ UTR was used with each ORF.

### Example 3—Optimized Orthogonal mRNAs Enable Increased Yields of Protein Containing Three Distinct ncAAs

Next, we demonstrated that the increase in protein expression yields from optimized O-mRNAs enables an increase in the yield of protein containing three distinct ncAAs, via orthogonal translation. As this work proceeded in parallel with the algorithm development described above, we performed our experiments with the best sequence available at the time, O1-ₛₜᵣₑₚGFP_{His6}, derived from the vol 1 algorithm (FIG. 2a). We created O1-ₛₜᵣₑₚGFP(40TAG, 136AGGA, 150AGTA)_{His6} and translated this with O-riboQ1 in cells containing a triply orthogonal PylRS/tRNA^{Pyl} pair (composed of MmPylRS/Methanosarcina spelaei (Mspe)tRNA^{Pyl}_{CUA} (which directs the incorporation of N⁶-(tert-butoxycarbonyl)-L-lysine (BocK) 1), Methanomassiliicoccus luminyensis 1 (Mlum)PylRS(NmH)/Methanomassiliicoccus intestinalis (Mint)tRNA^{Pyl-A17VC10}_{UCCU} (L121M, L125I, Y126F, M129A, V168V mutant, which directs the incorporation of Nπ-methyl-L-histidine (NmH) 2) and Methanomethylophilus sp. 1R26 (M1r26)PylRS(CbzK)/Methanomethylophilus alvus (Malv)tRNA^{Pyl-8}_{UACU} (Y126G, M129L mutant, which directs the incorporation of N⁶-((benzyloxy)carbonyl)-L-lysine (CbzK) 3). Full-length ₛₜᵣₑₚGFP(40BocK, 136NmH, 150CbzK)_{His6} was produced upon addition of BocK 1, NmH 2 and CbzK 3. Using this system, we synthesized 2.6±0.4 mg/L of ₛₜᵣₑₚGFP(40BocK, 136NmH, 150CbzK)_{His6}. This yield is 33 times greater than the yield from O(trans)-ₛₜᵣₑₚGFP(40TAG, 136AGGA or 150AGTA)_{His6} (FIG. 2b and Supplementary Table 2), corresponds to 9% of ₛₜᵣₑₚGFP(wt)_{His6} produced from O1-ₛₜᵣₑₚGFP(t)_{His6}, and to 11% of ₛₜᵣₑₚGFP(wt)_{His6} produced from ₛₜᵣₑₚGFP(wt)_{His6} translated from a wt RBS by wt ribosomes. The observed yields suggest a mean ncAA incorporation efficiency per step of 45%. Mass spectrometry confirmed the synthesis of the correct protein (FIG. 2c, Supplementary FIG. 2).

### Example 4—Design of Functional Operons for Quadruply Orthogonal aaRS/tRNA Pairs

Next, we aimed to build on the development of efficient O-mRNAs to enable the incorporation of four distinct ncAA into a single protein, with each ncAA encoded in response to a distinct quadruplet codon. This required four orthogonal aaRS/tRNA pairs that: (1) are mutually orthogonal in their aminoacylation specificity, (2) have four mutually orthogonal active sites, and (3) are assigned to four mutually orthogonal quadruplet codons. We chose a PylRS/tRNA^{Pyl} triplet—Methanomassiliicoccales archaeon RumEn M1 (Mrum)Pyl(NmH)RS/MinttRNA^{Pyl-A17VC10}_{UCCU} (L121M, L125I, Y126F, M129A, V168V mutant, which directs the incorporation of NmH 2), Methanogenic archaeon ISO4-G1 (Mg1)Pyl(CbzK)RS/MalvtRNA^{Pyl-8}_{UACU} (Y125G, M128L mutant, which directs the incorporation of CbzK 3) and MmPylRS/MspetRNA^{Pyl-evol}_{CUAG} (which directs the incorporation of several ncAAs, including BocK 1 or N⁶-((allyloxy)carbonyl)-L-lysine (AllocK) 4)—as a starting point for our approach. We chose the AfTyrRS(PheI)/AftRNA^{Try-A01}_{CUA}, (Y36I, L69M, H74L, Q116E, D165T, I166G, F274V, L298G, D299R mutant, which directs the incorporation of (S)-2-amino-3-(4-iodophenyl)propanoic acid (PheI) 5) as the starting point for a fourth aaRS/tRNA pair; we have previously shown that this pair is orthogonal to several pyrrolysyl synthetases and tRNA^{Pyl}s. Efforts to encode multiple ncAAs require strategies for the efficient and compact expression of the corresponding synthetases and tRNAs. We therefore established operon-based systems for the co-expression of the four exogenous tRNAs and the co-expression of their cognate synthetases.

In E. coli, many tRNAs are transcribed in polycistronic operons, and the 5′ and 3′ ends of mature tRNAs are generated by post-transcriptional RNase processing^{37, 38}. We created a program to automatically design synthetic tRNA operons in which the intergenic sequence between the exogenous tRNAs is derived from the sequence between E. coli tRNAs that are most similar to the exogenous tRNAs. The program first generates all possible orderings of the exogenous tRNAs. For each pair of adjacent exogenous tRNAs in an ordering, it identifies the adjacent natural tRNAs in the E. coli genome with the highest sequence identity to the exogenous pair. It then inserts the sequence of the intergenic region found between these natural tRNAs between the exogenous tRNAs. This process generates a synthetic operon sequence for each ordering of exogenous tRNAs. The program then compares the synthetic operons resulting from each tRNA order and ranks them based on the sum of the sequence identity between the exogenous tRNAs and the corresponding natural tRNAs used to define the intergenic regions in the operon.

We used our program for generating tRNA operons with AftRNA^{Ty-A01}, MspetRNA^{Pyl-evol}, MinttRNA^{Pyl-A17VC10}, and MalvtRNA^{Pyl-8}. The top ranked operon was: MinttRNA^{Pyl-A17VC10}_{UCCU}-inter(glyX, glyY)—MalvtRNA^{Pyl-8}_{UACU}-inter(glyW-cysT)-MspetRNA^{Pyl-evol}_{CUAG}-inter(argY, argZ)-AftRNA^{Tyr-A01}_{CUA}, where inter(x, y) represents the intergenic spacer sequence between the E. coli tRNAs x and y. To adapt this operon for expressing tRNAs that decode four distinct quadruplet codons we replaced MspetRNA^{Pyl-evol}_{CUAG} with MspetRNA^{Pyl-evol}_{UCUA} (created by transplanting an anticodon stem that we have previously evolved in MbtRNA^{Pyl} into MspetRNA^{Pyl}) and AftRNA^{Tyr-A01}_{CUA} by AftRNA^{Tyr-A01}_{CUAG} (created by anticodon mutation of AftRNA^{Tyr-A01}_{CUA}). We named the resulting tRNA operon tRNA4(quad).

To identify operons that would allow high expression of the four exogenous aaRSs (MmPylRS, AfTyr(PheI)RS, Mg1(CbzK)PylRS and Mrum(NmH)PylRS) we first generated five optimized 5′ UTR regions for each synthetase gene, and then predicted the ΔGₜₒₜ for going from the folded mRNA to the initiation competent translation complex for each 5′ UTR using any of the other three aaRS as 5′ sequence context. We chose two arrangements, RS4_1 and RS4_2, which had favorable ΔGₜₒₜ for all four aaRS. We cloned each of the aaRS operons into a plasmid encoding tRNA4 to generate compact synthetase and tRNA expression modules (RS4_1/tRNA4 and RS4_2/tRNA4) (Supplementary FIG. 3). We tested the activity of each aaRS in each operon (Supplementary FIG. 4, Supplementary Table 3). These experiments led us to design an optimized chimeric aaRS operon in which we transplanted 150 nt upstream of the optimised 5′ UTR of Mrum(NmH)PylRS from RS4_2 into RS4_1, creating RS4_1-2. This operon combined the best properties of RS4_2 and RS4_1 (Supplementary FIG. 4c).

We combined the RS4_1-2 and tRNA4(quad) operons in a single vector (279 RS4_1-2/tRNA4(quad)) and systematically tested the activity and orthogonality of each aaRS/tRNA pair produced by measuring the GFP fluorescence produced from O1-ₛₜᵣₑₚGFP(40XXXX)_{His6}, where XXXX stands for TAGA, AGGA, AGTA or CTAG. Cells contained O-riboQ1, each individual ncAA (NmH 2, CbzK 3, AllocK 4, PheI 5) or none, and RS4_1-2/tRNA4 (quad) (FIG. 3a-d). ESI-MS of ₛₜᵣₑₚGFP(40X)_{His6} (where X stands for NmH 2, CbzK 3, AllocK 4, Phel 5) produced by O-riboQ1 from O1-ₛₜᵣₑₚGFP(150XXXX)_{His6}, (where XXXX stands for TAGA, AGGA, AGTA or TAGA) in the presence of RS4_1-2/tRNA4(quad) and all four ncAAs (NmH 2, CbzK 3, PheI 5, AllocK 4) demonstrated that each aaRS, tRNA and codon are functionally orthogonal with respect to each other (FIG. 3e-h).

### Example 5—Genetically Encoding Four Distinct ncAAs Using Four Distinct Quadruplet Codons

We combined our advances in generating aaRS/tRNA operons for orthogonal pairs with our advances in creating optimized O-mRNAs, which are efficiently read by O-riboQ1, to incorporate four distinct ncAAs into a single protein in response to four distinct quadruplet codons (FIG. 4a). We produced strepGFP(40PheI, 50AllocK, 136NmH, 150CbzK)_{His6} by O-riboQ1 mediated translation of O1-ₛₜᵣₑₚGFP(40CTAG, 50TAGA, 136AGGA, 50AGTA)_{His6} in cells that contained RS4_1-2/tRNA4(quad) and were provided with all four ncAA substrates (NmH 2, CbzK 3, Phel 4, AllocK 5) (FIG. 4b). The production of ₛₜᵣₑₚGFP(40PheI, 50AllocK, 136NmH, 150CbzK)_{His6} was dependent upon the addition of all four ncAAs, and 0.41±0.03 mg/mL of the protein was produced (Supplementary Table 2). The observed yields suggest a mean ncAA incorporation efficiency per step of 38%. Mass spectrometry confirmed the incorporation of all four ncAAs in response to four distinct quadruplet codons (FIG. 4c, Supplementary FIG. 5). In additional experiments we also demonstrated the incorporation of four distinct ncAAs in response to three quadruplet codons and the amber codon (Supplementary FIG. 6-8, Supplementary Table 2).

### Example 6—Discussion of Examples 1 to 5

We have developed computational approaches to design O-mRNA sequences that are efficiently and selectively translated by O-ribosomes. The new O-mRNAs lead to up to 40-fold more protein, and are up to 50-fold more orthogonal, than O-mRNAs created by transplanting a previously used 5′ UTR containing the O-RBS in front of an ORF of interest. The O-mRNAs we created direct orthogonal protein production at levels comparable to—or greater than—those from the wt mRNAs translated by wt ribosomes. Our automated, rapid and scalable method for O-mRNA discovery will greatly accelerate the design and directed evolution of orthogonal translation systems that incorporate multiple ncAAs and polymerize new monomers^{3, 19-21} as well as the creation and application of orthogonal gene expression systems^{39, 40}.

Our O-mRNA optimization strategies include explicit selection for orthogonality and co-optimization of the 5′ UTR and ORF sequences. We found that co-optimizing the 5′ UTR and synonymous codon choices in the ORF led to O-mRNA sequences with predicted values for ΔGₜₒₜ(O-ribo) that are larger (more negative) than those obtained through simply varying the 5′ UTR; these sequences also have large (positive) predicted values for ΔGₜₒₜ (wt ribo). We discovered that co-optimization of the 5′ UTR sequence and synonymous codons within the ORF can improve protein yield, and testing four clones led to high levels of translation in each case tested. These observations are consistent with the view that mRNA folding is the major predictor—amongst known parameters—of protein yield²⁴. We note that other parameters, including codon adaptation, may influence protein yield, and it will be interesting to see whether including these considerations in future iterations of the algorithm will lead to even greater predictive power. Future work will also explore the co-optimisation of 5′ UTR sequences and coding sequence to improve production of difficult-to-express proteins from wt ribosomes.

By combining our automated O-mRNA design with our previously developed triply orthogonal PylRS/tRNA^{Pyl} pairs, we increased the yield of a protein containing three distinct ncAA 33-fold. We established a pipeline for the efficient and compact co-expression of many exogenous aaRS and tRNAs. We developed a computational program to produce polycistronic tRNA operons which mimic the endogenous transcription systems in E. coli. Our algorithm provides a general solution to produce multiple distinct tRNAs in E. coli under the same promoter on one plasmid and may be readily adapted for other organisms. We also devised polycistronic aaRS operons for the efficient expression of four mutually orthogonal synthetases alongside the tRNA operon. We combined our advances to produce a protein consisting of 24 amino acids—the canonical 20 amino acids and 4 ncAAs—in vivo for the first time. Each ncAAs is encoded using quadruplet codons, which are selectively translated on the O-mRNA and not used in natural translation, creating an organism with a 68-codon genetic code.

We anticipate that emerging developments in creating mutually orthogonal aaRS/tRNA pairs that recognize distinct ncAAs and decode distinct quadruplet codons may allow an expansion of the quadruplet code. The efficiency of quadruplet decoding may be further improved by selecting ribosomes that no longer read triplet codons or developing quadruplet decoding in organisms with compressed genetic codes, where competing triplet decoding tRNAs are removed^{6, 41}.

### REFERENCES FOR EXAMPLES 1 TO 6 AND FOR FIGURE LEGENDS 5 TO 12 (SUPPLEMENTARY FIGS. 1 TO 8)

1. Chin, J. W. Expanding and reprogramming the genetic code. Nature 550, 53-60 (2017).
2. de la Torre, D. & Chin, J. W. Reprogramming the genetic code. Nat. Rev. Genet., 1-16 (2020).
3. Neumann, H., Wang, K., Davis, L., Garcia-Alai, M. & Chin, J. W. Encoding multiple unnatural amino acids via evolution of a quadruplet-decoding ribosome. Nature 464, 441-444 (2010).
4. Wang, K. et al. Optimized orthogonal translation of unnatural amino acids enables spontaneous protein double-labelling and FRET. Nat. Chem. 6, 393-403 (2014).
5. Anderson, J. C. et al. An expanded genetic code with a functional quadruplet codon. Proc. Natl. Acad. Sci. U.S.A. 101, 7566-7571 (2004).
6. Fredens, J. et al. Total synthesis of Escherichia coli with a recoded genome. Nature 569, 514-518 (2019).
7. Wang, K. et al. Defining synonymous codon compression schemes by genome recoding. Nature 539, 59-64 (2016).
8. Malyshev, D. A. et al. A semi-synthetic organism with an expanded genetic alphabet. Nature 509, 385-388 (2014).
9. Zhang, Y. et al. A semi-synthetic organism that stores and retrieves increased genetic information. Nature 551, 644-647 (2017).
10. Zhang, Y. et al. A semisynthetic organism engineered for the stable expansion of the genetic alphabet. Proc. Natl. Acad. Sci. U.S.A. 114, 1317-1322 (2017).
11. Fischer, E. C. et al. New codons for efficient production of unnatural proteins in a semisynthetic organism. Nat. Chem. Biol. 16, 570-576 (2020).
12. Neumann, H., Slusarczyk, A. L. & Chin, J. W. De Novo Generation of Mutually Orthogonal Aminoacyl-tRNA Synthetase/tRNA Pairs. J. Am. Chem. Soc. 132, 2142-2144 (2010).
13. Chatterjee, A., Sun, S. B., Furman, J. L., Xiao, H. & Schultz, P. G. A Versatile Platform for Single- and Multiple-Unnatural Amino Acid Mutagenesis in Escherichia coli. Biochemistry 52, 1828-1837 (2013).
14. Willis, J. C. W. & Chin, J. W. Mutually orthogonal pyrrolysyl-tRNA synthetase/tRNA pairs. Nat. Chem. 10, 831-837 (2018).
15. Dunkelmann, D. L., Willis, J. C. W., Beattie, A. T. & Chin, J. W. Engineered triply orthogonal pyrrolysyl-tRNA synthetase/tRNA pairs enable the genetic encoding of three distinct non canonical amino acids. Nat. Chem. 12, 535-544 (2020).
16. Cervettini, D. et al. Rapid discovery and evolution of orthogonal aminoacyl-tRNA synthetase-tRNA pairs. Nat. Biotechnol. 38, 989-999 (2020).
17. Zhang, M. S. et al. Biosynthesis and genetic encoding of phosphothreonine through parallel selection and deep sequencing. Nat. Methods 14, 729-736 (2017).
18. Italia, J. et al. Mutually Orthogonal Nonsense-Suppression Systems and Conjugation Chemistries for Precise Protein Labeling at up to Three Distinct Sites. J. Am. Chem. Soc. 141, 6204-6212 (2019).
19. Rackham, O. & Chin, J. W. A network of orthogonal ribosome⋅mRNA pairs. Nat. Chen. Biol. 1, 159-166 (2005).
20. Wang, K., Neumann, H., Peak-Chew, S. Y. & Chin, J. W. Evolved orthogonal ribosomes enhance the efficiency of synthetic genetic code expansion. Nat. Biotechnol. 25, 770-777 (2007).
21. Schmied, W. H. et al. Controlling orthogonal ribosome subunit interactions enables evolution of new function. Nature 564, 444-448 (2018).
22. Venkat, S. et al. Genetically Incorporating Two Distinct Post-translational Modifications into One Protein Simultaneously. ACS Synth. Biol. 7, 689-695 (2018).
23. Chin, J. W. Expanding and Reprogramming the Genetic Code of Cells and Animals. Annu. Rev. Biochem. 83, 379-408 (2014).
24. Cambray, G., Guimaraes, J. C. & Arkin, A. P. Evaluation of 244,000 synthetic sequences reveals design principles to optimize translation in Escherichia coli. Nat. Biotechnol. 36, 1005-1015 (2018).
25. Plotkin, J. B. & Kudla, G. Synonymous but not the same: the causes and consequences of codon bias. Nat. Rev. Genet. 12, 32-42 (2011).
26. Tuller, T. & Zur, H. Multiple roles of the coding sequence 5′ end in gene expression regulation. Nucleic Acids Res. 43, 13-28 (2015).
27. Salis, H. M., Mirsky, E. A. & Voigt, C. A. Automated design of synthetic ribosome binding sites to control protein expression. Nat. Biotechnol. 27, 946-950 (2009).
28. Na, D., Lee, S. & Lee, D. Mathematical modeling of translation initiation for the estimation of its efficiency to computationally design mRNA sequences with desired expression levels in prokaryotes. BMC Syst. Biol. 4, 1-16 (2010).
29. Seo, S. W. et al. Predictive design of mRNA translation initiation region to control prokaryotic translation efficiency. Metab. Eng. 15, 67-74 (2013).
30. Salis, H. M. in Methods in Enzymology, Vol. 498 19-42 (Academic Press, Cambridge, MA, USA; 2011).
31. Espah Borujeni, A., Channarasappa, A. S. & Salis, H. M. Translation rate is controlled by coupled trade-offs between site accessibility, selective RNA unfolding and sliding at upstream standby sites. Nucleic Acids Res. 42, 2646-2659 (2014).
32. Espah Borujeni, A. & Salis, H. M. Translation Initiation is Controlled by RNA Folding Kinetics via a Ribosome Drafting Mechanism. J. Am. Chem. Soc. 138, 7016-7023 (2016).
33. Espah Borujeni, A. et al. Precise quantification of translation inhibition by mRNA structures that overlap with the ribosomal footprint in N-terminal coding sequences. Nucleic Acids Res. 45, 5437-5448 (2017).
34. Kudla, G., Murray, A. W., Tollervey, D. & Plotkin, J. B. Coding-Sequence Determinants of Gene Expression in Escherichia coli. Science 324, 255-258 (2009).
35. Allert, M., Cox, J. C. & Hellinga, H. W. Multifactorial Determinants of Protein Expression in Prokaryotic Open Reading Frames. J. Mol. Biol. 402, 905-918 (2010).
36. Goodman, D. B., Church, G. M. & Kosuri, S. Causes and Effects of N-Terminal Codon Bias in Bacterial Genes. Science 342, 475-479 (2013).
37. Phizicky, E. M. & Hopper, A. K. tRNA biology charges to the front. Genes Dev. 24, 1832-1860 (2010).
38. El Yacoubi, B., Bailly, M. & de Crécy-Lagard, V. Biosynthesis and Function of Posttranscriptional Modifications of Transfer RNAs. Annu. Rev. Genet. 46, 69-95 (2012).
39. An, W. & Chin, J. W. Synthesis of orthogonal transcription-translation networks. Proc. Natl. Acad. Sci. U.S.A. 106, 8477-8482 (2009).
40. Darlington, A. P. S., Kim, J., Jimenez, J. I. & Bates, D. G. Dynamic allocation of orthogonal ribosomes facilitates uncoupling of co-expressed genes. Nat. Commun. 9, 1-12 (2018).
41. Chatterjee, A., Lajoie, M. J., Xiao, H., Church, G. M. & Schultz, P. G. A Bacterial Strain with a Unique Quadruplet Codon Specifying Non-native Amino Acids. ChemBioChem 15, 1782-1786 (2014).

### Methods

### Thermodynamic Model of Translation Initiation

The thermodynamic model has been described previously¹. In brief, the model specifies the free energy difference, ΔGₜₒₜ, of the predicted energy of the free folded mRNA, ΔG_{unfolding}, and an initiation-competent ribosome-bound state, ΔG_{ribo_binding}.

$$Δ {G}_{tot} = Δ {G}_{ribo \_ binding} + Δ {G}_{unfolding}$$

Here, ΔG_{unfolding} is the energy required to unfold mRNA secondary structures. The free energy released on formation of the initiation-competent state, ΔG_{ribo_binding}, consists of four components.

$$Δ {G}_{ribo binding} = Δ {G}_{mRNA - rRNA} + Δ {G}_{start} + Δ {G}_{spacing} - Δ {G}_{standby}$$

ΔG_{mRNA-rRNA} is the free energy of the predicted co-folded secondary structure of the last 9 nt of the 16S rRNA and the mRNA, in which the main energetic contribution comes from the hybridization energy between the mRNA's Shine Dalgarno (SD) or orthogonal Shine Dalgarno O-SD sequence and the 16S rRNA. mRNA folding downstream of the hybridization site is not permitted, reflecting the ribosomal footprint. ΔGₛₜₐᵣₜ is the energy released from the binding of the initiator tRNA to the start codon. ΔG_{spacing} is an energy penalty for non-optimal spacing length between the SD site and the start codon. ΔG_{standby} is the energy required to unfold secondary structures that sequester the standby site, which is here defined as the four nucleotides upstream of the SD site.

### Simulated Annealing Optimization Algorithm for Automated O-mRNA Discovery

RNA secondary structure predictions are performed in the NuPACK suite using the ‘mfe’ algorithm. The calculations consider a window of at most 35 nt in the 5′ UTR and ORF; if longer sequences are used, only the 35 nt closest to the start codon are considered.

The vol 1 algorithm is derived from a previously described simulated annealing optimization algorithm 1, but using the final 9 nt of the orthogonal 16S rRNA (ATGGGATTA) instead of the canonical sequence (ACCTCCTTA) for the calculation of ΔG_{mRNA-rRNA}. In brief, the algorithm starts from a random 5′ UTR sequence containing a canonical SD sequence. The ΔGₜₒₜ(O-ribo) of the 5′ UTR and the ORF is evaluated using the thermodynamic model and compared to a target function ΔG_{target}. The ΔG_{target} may be set to an arbitrarily or infinitely negative value such that the target of the algorithm is as negative as possible. In an iterative procedure, a mutation (either a single nucleotide change, an insertion or a deletion) is introduced into the 5′ UTR and a new ΔGₜₒₜ^{new}(O-ribo) is calculated. If the mutated sequence violates sequence constraints, the mutation is rejected. If the mutated sequence leads to a ΔGₜₒₜ^{new}(O-ribo) closer to ΔG_{target}, the mutation is accepted. If the ΔGₜₒₜ^{new}(O-ribo) value is more different from to ΔG_{target} than the original ΔGₜₒₜ(O-ribo), the mutation is accepted with a probability of

$$exp ( \frac{\vert Δ {G}_{tot}^{new} ( O - ribo ) - Δ {G}_{tot} ( O - ribo ) \vert}{{T}_{SA}} )$$

Here, T_{SA} is the simulated annealing temperature, which is adjusted to maintain a 5-20% acceptance rate. The algorithm terminates after 10,000 iterations and outputs the 5′ UTR and predicted ΔGₜₒₜ(O-ribo).

The vol 2 algorithm builds on the vol 1 algorithm. The random starting 5′ UTR contains the 9 nucleotide O-SD site (TAATCCCAT) which is predicted to be perfectly complementary to the O-16S rRNA (ATGGGATTA) at an optimal spacing of 5 nucleotides from the ATG start codon. The ΔGₜₒₜ(wt ribo) and ΔGₜₒₜ(O-ribo) of the 5′ UTR and the ORF are evaluated using the thermodynamic model, and a hypothetical ΔGₜₒₜ(opt) is calculated according to ΔGₜₒₜ(opt)=ΔGₜₒₜ(O-ribo)−0.5*ΔGₜₒₜ(wt ribo). In contrast to the vol 1 algorithm, no ΔG_{target} value is specified. In an iterative procedure, a mutation (either a single nucleotide change, an insertion or a deletion) is introduced into the 5′ UTR and new ΔGₜₒₜ^{new} values are calculated. If the mutated sequence violates sequence constraints or removes the 5 nucleotide core of the O-SD sequence (TCCCA), the mutation is rejected. If the mutated sequence leads to an improved (more negative) ΔGₜₒₜ^{new}(opt) value, the mutation is accepted. If the ΔGₜₒₜ^{new}(opt) value is greater (more positive) than the original ΔGₜₒₜ(opt), the mutation is accepted with a probability of

$$exp ( \frac{\vert Δ {G}_{tot}^{new} ( opt ) - Δ {G}_{tot} ( opt ) \vert}{{T}_{SA}} )$$

If 500 consecutive iterations yield no improvements in ΔGₜₒₜ(opt), the algorithm terminates and outputs the 5′ UTR and ΔGₜₒₜ values. We typically run the algorithm multiple times and select sequences with the most favourable ΔGₜₒₜ values; we found this is computationally more efficient to identify highly translated 5′ UTRs than running the algorithm for more iterations per starting sequence. In this work, we chose 4 sequences out of 24 predicted 5′ UTRs.

The vol 3 algorithm builds on the vol 2 algorithm. In addition to the random starting 5′ UTR, the amino acids at positions 2 to 12 are encoded by a randomly selected choice of synonymous codons. Synonymous codon changes in positions 2 to 12 in the ORF, in addition to a single nucleotide change, insertion, or deletion in the 5′ UTR, are permitted as a mutation mechanism during the simulated annealing optimization.

tRNA Operon Designer

The program generates a list of all pairs of tRNAs in the host organism whose genes are adjacent to one another and on the same strand. It then extracts the gene sequences of these endogenous tRNA pairs as well as the corresponding intergenic sequences. Optionally, the user may specify minimum and maximum lengths of intergenic sequences to be considered by the program. For the tRNA operons used in this work, we used the E. coli strain K-12 substrain MG1655 genome (version U00096.3, last modified 24 Sep. 2018) as the host genome, with minimum and maximum intergenic sequence lengths of 10 and 100 base pairs, respectively.

Next, the program generates all ordered pairs of the exogenous tRNAs. For each ordered pair of exogenous tRNAs, the acceptor stem sequences of these tRNAs are compared with the acceptor stem sequences of the endogenous tRNA pairs. For consistency, we consider the first seven and last eight nucleotides of the tRNAs (excluding the CCA end), which comprise the canonical E. coli tRNA acceptor stem and discriminator base region. Each endogenous tRNA pair ranked by similarity to the exogenous tRNA pair, calculated as the sequence identity of the acceptor stems. The exogenous tRNA pair is then assigned a score, defined as the sequence identity of the acceptor stems of the most similar endogenous tRNA pair.

Finally, the program generates all orderings, or permutations, of the exogenous tRNAs. Synthetic tRNA operons corresponding to each permutation are created by inserting endogenous tRNA intergenic regions between each ordered pair of exogenous tRNA genes in the permutation. For each ordered exogenous pair, the intergenic region corresponding to the most similar endogenous tRNA pair is chosen. Each operon is assigned a score, calculated as the sum of the scores of all the ordered pairs in the permutation.

The sequences and scores of the operons, along with information about the order of the tRNAs and the intergenic regions chosen, are presented as a ranked list of entries in an Office Open XML spreadsheet.

aaRS Operon Assembly

Details for the operon assembly are given in Supplementary FIG. 3. All predicted 5′ UTRs with ΔGₜₒₜ (wt ribo) for the alignments are given in Supplementary Table 3.

### DNA Constructs

Reporter genes (ₛₜᵣₑₚGFP_{His6}, mCherry and E2Crimson) were cloned by Gibson assembly into a p15A plasmid containing a tetracycline resistance cassette and were expressed from a lac promoter. Optimised 5′ UTRs were inserted between the +1 transcription site and the ORF by quick-change PCR Gibson assembly. Optimised 5′ UTRs and ORFs were inserted between the +1 transcription site and codon 13 by quick change PCR Gibson assembly. O(trans)-ₛₜᵣₑₚGFP(40TAG, 136AGGA, 150AGTA)_{His6} was expressed from a previously described p15A plasmid2. O1-ₛₜᵣₑₚGFP(40TAG, 136AGGA, 150AGTA)_{His6}, O1-ₛₜᵣₑₚGFP(40TAG, 50CTAG, 136AGGA, 150AGTA)_{His6} and O1-ₛₜᵣₑₚGP(40CTAG, 50AGA, 136AGGA, 150AGTA)_{His6} were synthesized by IDT as gBlock double-stranded DNA fragments and cloned into the standard p15A reporter backbone by Gibson assembly. Ribosomes were encoded on previously described pRSF plasmids containing a kanamycin resistance cassette and were expressed from a trc promoter^{3,4}.

Synthetase operon RS3 and tRNA operon tRNA3 were encoded on a previously described pMB1 plasmid containing a spectinomycin resistance cassette. Synthetase operons RS4_1 and RS4_2 were synthesized by IDT as gBlocks and inserted after the +1 transcription site of a glnS′ promoter by Gibson assembly². RS4_1-2 was assembled by Gibson cloning of fragments from RS4_1 and RS4_2. tRNA operon tRNA4 was synthesized by IDT as a gBlock and assembled into the same pMB1 plasmid as the synthetase operons by Gibson cloning under control of a lpp promoter. tRNA4(quad) was assembled by quick change PCR Gibson assembly from tRNA4.

### Measuring the Activity and Orthogonality of Fluorescent Reporters

To measure the activity and orthogonality of each fluorescent reporter (ₛₜᵣₑₚGFP_{His6}, mCherry and E2Crimson) we transformed 0.5 μL of p15A plasmids encoding the fluorescent reporter into 8 μL chemically competent E. coli DH10B cells bearing a pRSF plasmid encoding a copy of the O-ribosome or wt ribosome. We recovered the transformed cells for 1 h at 37° C. and 750 rpm in 180 μL SOC medium in a 96-well microtiter plate format. 30 μL of the rescued cells were used to inoculate 500 μL selective 2xYT-kt (2xYT medium containing 50 μg/mL kanamycin, 12.5 μg/mL tetracycline) medium in a 1.2 mL 96-well plate format and the cultures were grown over night at 37° C. and 750 rpm. 30 μL of the overnight cultures were used to inoculate 500 μL 2xYT-kt medium in a 1.2 mL 96-well plate format. Cells were grown for 2 h at 37° C. and 750 rpm and production of fluorescent reporter as well as ribosome was induced by addition of 10 μL 0.1 M IPTG to give a final concentration of 2 mM IPTG. Cells were grown for 18 h at 37° C. and 750 rpm. 180 μL of each culture was transferred into 96-well flat bottom Costar plates and fluorescence and optical density were measured using a PHERAstar FS plate reader.

Comparative Analysis of Efficiency of Triple Incorporation from O1-ₛₜᵣₑₚGFP(40TAG, 136AGGA, 150AGTA)_{His6} or O(trans)-ₛₜᵣₑₚGFP(40TAG, 136AGGA, 150AGTA)_{His6}

To compare the efficiency of the incorporation of three distinct ncAAs into ₛₜᵣₑₚGFP(40TAG, 136AGGA, 150AGTA)_{His6} from reporters containing a transplanted or optimised orthogonal 5′UTR we transformed 0.4 μL pMB1 plasmid encoding operon RS3/tRNA3 together with 0.4 μL of p15A plasmid encoding O1-ₛₜᵣₑₚGFP_{His6}, O1-ₛₜᵣₑₚGFP(40TAG, 136AGGA, 150AGTA)_{His6} or O(trans)-ₛₜᵣₑₚGFP(40TAG, 136AGGA, 150AGTA)_{His6} into 8 μL chemically competent E. coli DH10B cells bearing a pRSF plasmid encoding a copy of O-riboQ1. We recovered the transformed cells for 1 h at 37° C. and 750 rpm in 180 μL SOC medium in a 96-well microtiter plate format. 30 μL of the rescued cells were used to inoculate 500 μL 2xYT-kts medium (2xYT containing 25 μg/mL kanamycin, 12.5 μg/mL tetracyclin and 37.5 μg/mL spectinomycin) in a 1.2 mL 96-well plate format and the cultures were grown over night at 37° C. and 750 rpm. 100 μL of the overnight cultures were used to inoculate 4 mL 2xYT-kts medium containing either 4 mM BocK 1, 4 mM NmH 2 and 2 mM CbzK 3 or no ncAA in a 10 mL 24-well plate format. Cells were grown for 2 h at 37° C. and 220 rpm and production of ₛₜᵣₑₚGFP_{His6} as well as O-riboQ1 was induced by addition of 8 μL 1 M IPTG to give a final concentration of 2 mM IPTG. Cells were grown for 18 h at 37° C. and 750 rpm. 180 μL of each culture was transferred into 96-well flat bottom Costar plates and fluorescence and optical density were measured using PHERAstar FS. The rest of the cultures were centrifuged for 10 min at 3200 rcf and taken up in OD600 adjusted amounts of BugBuster containing Roche cOmplete proteinase inhibitor. Cells were lysed for 1 h under head-over-tail rotation at room temperature. The lysate was transferred into 1.5 mL Eppendorf tubes and spun down at 15000 rcf for 20 min. 180 μL of clarified cell lysate was transferred into 96-well flat bottom Costar plates and fluorescence and was measured using PHERAstar FS.

Activity and Orthogonality Assessment of aaRS/tRNA Operons

To assess the activity and orthogonality of each aaRS/tRNA pair in our operons we transformed 0.4 μL pMB1 plasmids encoding operons (aaRS4_1/tRNA4, aaRS4_2/tRNA4, aaRS4_1-2/tRNA4 and aaRS4_1-2/tRNA(quad)) into 8 μL chemically competent E. coli DH10B cells harbouring a pRSF plasmid encoding a copy of O-riboQ1 as well as a p15A plasmid encoding O1-ₛₜᵣₑₚGFP(40XXXX)_{His6}, where XXXX stands for either TAG (with all operons but aaRS4_1-2/tRNA4(quad)), TAGA (only with aaRS4_1-2/tRNA4(quad)), AGGA, AGTA or CTAG. We recovered the transformed cells for 1 h at 37° C. and 750 rpm in 180 μL SOC medium in a 96-well microtiter plate format. 30 μL of the rescued cells were used to inoculate 500 μL selective 2xYT-kts medium in a 1.2 mL 96-well plate format and the cultures were grown over night at 37° C. and 750 rpm. 30 μL of the overnight cultures were used to inoculate 500 μL selective 2xYT-kts medium containing either 4 mM BocK 1, 4 mM NmH 2, 2 mM CbzK 3, 4 mM AllocK 4, 2 mM Phel 5 or no ncAA in a 1.2 mL 96-well plate format. Cells were grown for 2 h at 37° C. and 750 rpm and expression of ₛₜᵣₑₚGFP(40XXXX)_{His6} as well as O-riboQ1 was induced by addition of 10 μL 0.1 M IPTG to give a final concentration of 2 mM IPTG. Cells were grown for 18 h at 37° C. and 750 rpm. 180 μL of each culture was transferred into 96-well flat bottom Costar plates and fluorescence and optical density were measured using PHERAstar FS.

Production of ₛₜᵣₑₚGFP(40X)_{His6} for MS Analysis

To isolate proteins for MS analysis to assess the orthogonality of the aaRS/tRNA operons 0.4 μL pMB1 plasmid encoding operon RS4_1-2/tRNA4 or RS4_1-2/tRNA4(quad) together with 0.4 μL p15A plasmid encoding either O1-ₛₜᵣₑₚGFP(40XXXX)_{His6}, where XXXX stands for TAG (only with RS4_1-2/tRNA4), TAGA (only with RS4_1-2/tRNA4(quad)), AGGA, AGTA and CTAG respectively into 50 μL chemically competent E. coli DH10B cells harbouring a pRSF plasmid encoding a copy of O-riboQ1. We recovered the transformed cells for 1 h at 37° C. and 750 rpm in 400 μL SOC medium in a 1.5 mL Eppendorf tube. 100 μL of the rescued cells were used to inoculate 50 mL selective 2xYT-kts medium in a 250 mL Erlenmeyer flask and the cultures were grown over night at 37° C. and 220 rpm. 5 mL of the overnight cultures were used to inoculate 100 mL selective 2xYTkts medium containing a combination of ncAAs BocK 1, NmH 2, CbzK 3, AllocK 4 and PheI 5 according to the constructs used (RS4_1-2/tRNA4 with 1, 2, 3, 5-RS4_1-2/tRNA4(quad) 2, 3, 4, 5). Cultures were grown for 2-3 h at 37° C. and 220 rpm until OD₆₀₀ 0.5 and induced with 200 μL 1 M IPTG to a final concentration of 2 mM IPTG. Cells were grown at 37° C. and 220 rpm for 18 h. Cells were centrifuged at 3200 rcf for 12 min, resuspended in 10 mL BugBuster containing Roche cOmplete proteinase inhibitor, sonicated for 1.5 min (2 s on 2 s off at 40% amplitude) and the lysate was centrifuged for 20 min at 15000 rcf at 4° C. The lysate was bound to 40 μL nickel NTA beads overnight. Beads were washed six times with 240 μL 20 mM imidazole in PBS. Proteins were eluted 9 times in 20 μL 250 mM imidazole. The buffer was exchanged for water using a 3 kDa Amicon ultra column for MS and MS/MS analysis.

Orthogonality and Efficiency Assessment of the Incorporation of Four Distinct ncAAs in Response to Four Distinct Quadruplet Codons from O-ₛₜᵣₑₚGFP(40CTAG, 50TAGA, 136AGGA, 150AGTA)_{His6}

To assess the efficiency and orthogonality of the incorporation of four distinct ncAAs into four distinct quadruplet codons we transformed 0.4 μL pMB1 plasmid encoding operon RS4_1-2/tRNA4(quad) together with 0.4 μL p15A plasmid encoding either O1-ₛₜᵣₑₚGFP_{His6} or O1-ₛₜᵣₑₚGFP(40CTAG, 50TAGA, 136AGGA, 150AGTA)_{His6} into 8 μL chemically competent E. coli DH10B cells bearing a pRSF plasmid encoding a copy of O-riboQ1. We recovered the transformed cells for 1 h at 37° C. and 750 rpm in 180 μL SOC medium in a 96-well microtiter plate format. 30 μL of the rescued cells were used to inoculate 500 μL selective 2xYT-kts medium in a 1.2 mL 96-well plate format and the cultures were grown over night at 37° C. and 750 rpm. 100 μL of the overnight cultures were used to inoculate 4 mL selective 2xYT-kts medium containing either each combination of three out of the four ncAAs: 4 mM NmH 2, 2 mM CbzK 3, 4 mM AllocK 4 and 2 mM PheI 5, all ncAAs or none (O1-ₛₜᵣₑₚGFP_{His6} was only grown in presence of all ncAAs) in a 24-well plate format. Cells were grown for 2 h at 37° C. and 220 rpm and production of ₛₜᵣₑₚGFP_{His6} as well as O-riboQ1 was induced by addition of 8 μL 1 M IPTG to give a final concentration of 2 mM IPTG. Cells were grown for 18 h at 37° C. and 750 rpm. 180 μL of each culture was transferred into 96-well flat bottom costar plates and fluorescence and optical density were measured using PHERAstar FS.

The same procedure was used for the orthogonality and efficiency assessment of the incorporation of four distinct ncAAs in response to one amber codon and three distinct quadruplet codons into O1-ₛₜᵣₑₚGFP(40TAG, 50CTAG, 136AGGA, 150AGTA)_{His6}. However, RS4_1-2/tRNA4 was used as operon and O1-ₛₜᵣₑₚGFP(40TAG, 50CTAG, 136AGGA, 150AGTA)_{His6} as reporter for quadruplet incorporation. 4 mM BocK 1 was used instead of 4 mM AllocK 4.

Production of ₛₜᵣₑₚGFP(XXXX)_{His6} for MS Analysis and Determination of Isolated Yield of the Incorporation of Three and Four Distinct ncAAs

The same procedure for the protein production as for mass spectrometry analysis of ₛₜᵣₑₚGFP(XXXX)_{His6} was used with the following combinations of reporters, operons and ncAAs: O1-ₛₜᵣₑₚGFP(40TAG, 136AGGA, 150AGTA)_{His6} with RS3/tRNA3 and 4 mM BocK 1, 4 mM NmH 2, 2 mM CbzK 3 or O₁-ₛₜᵣₑₚpGFP(40TAG, 50CTAG, 136AGGA, 150AGTA)_{His6} with RS4_1-2/tRNA4 and 4 mM BocK 1, 4 mM NmH 2, 2 mM CbzK 3, 2 mM PheI 5 or O1-ₛₜᵣₑₚGFP(40CTAG, 50TAGA, 136AGGA, 150AGTA)_{His6} with RS4_1-2/tRNA4(quad) and 4 mM NmH 2, 2 mM CbzK 3, 4 mM AllocK 4, 2 mM PheI 5.

To determine the isolated yield fluorescence of 180 μL isolated protein was measured using PHERAstar FS and the protein concentration was calculated based on a standard curve generated with a ₛₜᵣₑₚGFP_{His6} standard. The buffer was exchanged for water using a 3 kDa Amicon ultra column for MS and MS/MS analysis.

### Electrospray Ionization Mass Spectrometry

Denatured protein samples (˜10 μM) were subjected to LC-MS analysis. Briefly, proteins were separated on a C4 BEH 1.7 μm, 1.0×100 mm UPLC column (Waters, UK) using a modified nanoAcquity (Waters, UK) to deliver a flow of approximately 50 l/min. The column was developed over 20 minutes with a gradient of acetonitrile (2% v/v to 80% v/v) in 0.1% v/v formic acid. The analytical column outlet was directly interfaced via an electrospray ionisation source, with a hybrid quadrupole time-of-flight mass spectrometer (Xevo G2, Waters, UK). Data was acquired over a m/z range of 300-2000, in positive ion mode with a cone voltage of 30V. Scans were summed together manually and deconvoluted using MaxEnt1 (Masslynx, Waters, UK). The theoretical molecular weights of proteins with ncAAs was calculated by first computing the theoretical molecular weight of wild-type protein using an online tool (http://web.expasy.org/protparam/) and then manually correcting for the theoretical molecular weight of ncAAs.

### Tandem MS/MS Analysis

Proteins were run on 4-12% NuPAGE Bis-Tris gel (Invitrogen) with MES buffer and briefly stained using InstantBlue (Expedeon). The bands were excised and stored in water.

Tryptic digestion and tandem MS/MS analyses were done by Mark Skehel (Biological Mass Spectrometry and Proteomics Laboratory, MRC Laboratory of Molecular Biology).

### REFERENCES FOR METHODS SECTION

1. Salis, H. M., Mirsky, E. A. & Voigt, C. A. Automated design of synthetic ribosome binding sites to control protein expression. Nat. Biotechnol. 27, 946-950, doi:10.1038/nbt.1568 (2009).
2. Dunkelmann, D. L., Willis, J. C. W., Beattie, A. T. & Chin, J. W. Engineered triply orthogonal pyrrolysyl-tRNA synthetase/tRNA pairs enable the genetic encoding of three distinct non-canonical amino acids. Nat. Chem. 12, 535-544, doi:10.1038/s41557-020-0472-x (2020).
3. Neumann, H., Wang, K., Davis, L., Garcia-Alai, M. & Chin, J. W. Encoding multiple unnatural amino acids via evolution of a quadruplet-decoding ribosome. Nature 464, 441-444, doi:10.1038/nature08817 (2010).
4. Wang, K. et al. Optimized orthogonal translation of unnatural amino acids enables spontaneous protein double-labelling and FRET. Nat. Chem. 6, 393-403, doi:10.1038/nchem.1919 (2014).

SUPPLEMENTARY TABLE 1

| Name | | | Predicted energy - orthogonal ribosome | | | | | Predicted energy - wt ribosome | |
|---|---|---|---|---|---|---|---|---|---|
| Protein | Mode | Sequence | o_dG_total | o_mRNA_rRNA | o_mRNA | o_spacing | o_standby | wt_dG_total | wt_mRNA_rRNA |
| StrepGFPHis6 | | wt | 20.3 | −5.3 | −14.6 | 12.2 | 0.0 | −0.5 | −14.6 |
| StrepGFPHis6 | | trans | 3.5 | −8.0 | −12.7 | 0.0 | 0.0 | 22.8 | −1.9 |
| StrepGFPHis [symbol] | Vol 1 | seq1 | −5.8 | −14.1 | −9.5 | 0.0 | 0.0 | 16.3 | −6.4 |
| StrepGFPHis [symbol] | Vol 1 | seq2 | −4.9 | −15.5 | −11.8 | 0.0 | 0.0 | 8.4 | −2.2 |
| StrepGFPHis [symbol] | Vol 1 | seq3 | −5.1 | −14.7 | −10.8 | 0.0 | 0.0 | 7.6 | −2.0 |
| StrepGFPHis [symbol] | Vol 1 | seg4 | −6.6 | −14.6 | −8.4 | 0.0 | −0.8 | 15.2 | −1.8 |
| StrepGFPHis [symbol] | Vol 2 | seq1 | −7.6 | −19.5 | −13.1 | 0.0 | 0.0 | 26.3 | −7.5 |
| StrepGFPHis [symbol] | Vol 2 | seq2 | −7.9 | −14.5 | −7.8 | 0.0 | 0.0 | 28.7 | −8.8 |
| StrepGFPHis [symbol] | Vol 2 | seq3 | −7.2 | −19.2 | −12.9 | 0.3 | 0.0 | 13.6 | −10.7 |
| StrepGFPHis [symbol] | Vol 2 | seg4 | −8.0 | −14.3 | −7.5 | 0.0 | 0.0 | 29.3 | −3.2 |
| StrepGFPHis [symbol] | Vol 3 | seq1 | −12.4 | −14.3 | −2.8 | 0.3 | 0.0 | 28.4 | −4.1 |
| StrepGFPHis [symbol] | Vol 3 | seq2 | −12.9 | −17.1 | −5.4 | 0.0 | 0.0 | 29.5 | −8.1 |
| StrepGFPHis [symbol] | Vol 3 | seq3 | −12.5 | −14.8 | −3.2 | 0.3 | 0.0 | 29.3 | −3.6 |
| StrepGFPHis [symbol] | Vol 3 | seq4 | −12.8 | −17.0 | −5.1 | 0.3 | 0.0 | 29.0 | −8.3 |
| mCherry | | wt | 15.6 | −5.3 | −9.9 | 12.2 | 0.0 | −5.2 | −14.6 |
| mCherry | | trans | 3.9 | −8.0 | −13.1 | 0.0 | 0.0 | 23.2 | −1.9 |
| mCherry | Vol 2 | seq1 | −9.9 | −14.4 | −5.7 | 0.0 | 0.0 | 29.9 | −5.5 |
| mCherry | Vol 2 | seq2 | −9.9 | −13.5 | −4.8 | 0.0 | 0.0 | 29.9 | −4.6 |
| mCherry | Vol 2 | seq3 | −8.8 | −17.3 | −9.0 | 0.7 | 0.0 | 29.9 | −6.4 |
| mCherry | Vol 2 | seq4 | −9.9 | −19.0 | −10.3 | 0.0 | 0.0 | 29.4 | −8.2 |
| mCherry | Vol 3 | seq1 | −13.7 | −17.1 | −4.6 | 0.0 | 0.0 | 9.4 | −7.7 |
| mCherry | Vol 3 | seq2 | −13.7 | −18.3 | −5.8 | 0.0 | 0.0 | 29.9 | −8.1 |
| mCherry | Vol 3 | seq3 | −13.0 | −20.4 | −8.6 | 0.0 | 0.0 | 20.9 | −8.4 |
| mCherry | Vol 3 | seq4 | −13.6 | −21.6 | −9.2 | 0.0 | 0.0 | 8.2 | −13.3 |
| E2Crimson | | wt | 20.2 | −5.3 | −14.5 | 12.2 | 0.0 | −0.6 | −14.6 |
| E2Crimson | | trans | 0.3 | −8.0 | −9.5 | 0.0 | 0.0 | 19.6 | −1.9 |
| E2Crimson | Vol 2 | seq1 | −8.4 | −19.7 | −12.5 | 0.0 | 0.0 | 28.4 | −9.1 |
| E2Crimson | Vol 2 | seq2 | −8.7 | −16.7 | −8.9 | 0.3 | 0.0 | 29.6 | −9.0 |
| E2Crimson | Vol 2 | seq3 | −9.1 | −17.2 | −8.6 | 0.7 | 0.0 | 20.3 | −3.3 |
| E2Crimson | Vol 2 | seq4 | −9.5 | −12.8 | −4.5 | 0.0 | 0.0 | 28.7 | −5.5 |
| E2Crimson | Vol 3 | seq1 | −13.2 | −15.0 | −3.0 | 0.0 | 0.0 | 29.7 | −5.5 |
| E2Crimson | Vol 3 | seq2 | −13.2 | −13.2 | −1.2 | 0.0 | 0.0 | 7.6 | −4.6 |
| E2Crimson | Vol 3 | seq3 | −13.2 | −14.7 | −2.7 | 0.0 | 0.0 | 29.4 | −5.5 |
| E2Crimson | Vol 3 | seq4 | −13.2 | −21.4 | −9.4 | 0.0 | 0.0 | 28.1 | −8.6 |

| Name | | | Predicted energy - wt ribosome | | | Experimental | |
|---|---|---|---|---|---|---|---|
| Protein | Mode | Sequence | wt_mRNA | wt_spacing | wt_standby | o_RFU_observed | wt_RFU_observed |
| StrepGFPHis6 | | wt | −14.6 | 0.7 | 0.0 | 6172 | 11302 |
| StrepGFPHis6 | | trans | −12.7 | 9.6 | −3.5 | 468 | 391 |
| StrepGFPHis [symbol] | Vol 1 | seq1 | −9.5 | 14.4 | 0.0 | 14378 | 748 |
| StrepGFPHis [symbol] | Vol 1 | seq2 | −11.8 | 0.0 | 0.0 | 5198 | 358 |
| StrepGFPHis [symbol] | Vol 1 | seq3 | −10.8 | 0.0 | 0.0 | 6915 | 365 |
| StrepGFPHis [symbol] | Vol 1 | seg4 | −8.4 | 9.8 | 0.0 | 10687 | 475 |
| StrepGFPHis [symbol] | Vol 2 | seq1 | −13.1 | 21.9 | 0.0 | 1556 | 316 |
| StrepGFPHis [symbol] | Vol 2 | seq2 | −7.8 | 30.9 | 0.0 | 8525 | 390 |
| StrepGFPHis [symbol] | Vol 2 | seq3 | −12.9 | 7.2 | −5.4 | 408 | 308 |
| StrepGFPHis [symbol] | Vol 2 | seg4 | −7.5 | 26.2 | 0.0 | 7507 | 391 |
| StrepGFPHis [symbol] | Vol 3 | seq1 | −2.8 | 30.9 | 0.0 | 2569 | 359 |
| StrepGFPHis [symbol] | Vol 3 | seq2 | −5.4 | 33.4 | 0.0 | 3494 | 326 |
| StrepGFPHis [symbol] | Vol 3 | seq3 | −3.2 | 30.9 | 0.0 | 5961 | 371 |
| StrepGFPHis [symbol] | Vol 3 | seq4 | −5.1 | 33.4 | 0.0 | 19134 | 511 |
| mCherry | | wt | −9.9 | 0.7 | 0.0 | 20184 | 33818 |
| mCherry | | trans | −13.1 | 9.6 | −3.5 | 2438 | 1225 |
| mCherry | Vol 2 | seq1 | −5.7 | 30.9 | 0.0 | 13387 | 2320 |
| mCherry | Vol 2 | seq2 | −4.8 | 30.9 | 0.0 | 25326 | 1632 |
| mCherry | Vol 2 | seq3 | −9.0 | 28.5 | 0.0 | 20777 | 2882 |
| mCherry | Vol 2 | seq4 | −10.3 | 28.5 | 0.0 | 3716 | 1594 |
| mCherry | Vol 3 | seq1 | −4.6 | 9.8 | −3.9 | 19994 | 423 |
| mCherry | Vol 3 | seq2 | −5.8 | 33.4 | 0.0 | 19296 | 276 |
| mCherry | Vol 3 | seq3 | −8.6 | 21.9 | 0.0 | 75482 | 768 |
| mCherry | Vol 3 | seq4 | −9.2 | 5.0 | −8.5 | 49971 | 658 |
| E2Crimson | | wt | −14.5 | 0.7 | 0.0 | 57385 | 68560 |
| E2Crimson | | trans | −9.5 | 9.6 | −3.5 | 4708 | 1131 |
| E2Crimson | Vol 2 | seq1 | −12.5 | 26.2 | 0.0 | 67890 | 1871 |
| E2Crimson | Vol 2 | seq2 | −8.9 | 30.9 | 0.0 | 7404 | 1116 |
| E2Crimson | Vol 2 | seq3 | −8.6 | 16.1 | 0.0 | 4649 | 1114 |
| E2Crimson | Vol 2 | seq4 | −4.5 | 30.9 | 0.0 | 36823 | 1361 |
| E2Crimson | Vol 3 | seq1 | −3.0 | 33.4 | 0.0 | 4560 | 1088 |
| E2Crimson | Vol 3 | seq2 | −1.2 | 12.2 | 0.0 | 7316 | 1118 |
| E2Crimson | Vol 3 | seq3 | −2.7 | 33.4 | 0.0 | 53309 | 2463 |
| E2Crimson | Vol 3 | seq4 | −9.4 | 28.5 | 0.0 | 41689 | 1618 |

[symbol]

indicates data missing or illegible when filed

SUPPLEMENTARY TABLE 2
primary data for linear regression of fluorescence v. concentration

| | | | | Replicate 3 | |
|---|---|---|---|---|---|
| Replicate 1 | | Replicate 2 | | | strepGFPHis6 |
| μg/mL | strepGFPHis [symbol] | μg/mL | strepGFPHis [symbol] | μg/mL | fluorescence (au) |
| 175 | 48555 | 147.058824 | 41362 | 163.235294 | 46491 |
| 87.5 | 25355 | 73.5294118 | 22506 | 81.6176471 | 23892 |
| 43.75 | 12876 | 36.7647059 | 11093 | 40.8088235 | 12401 |
| 21.875 | 6454 | 18.3823529 | 5578 | 20.4044118 | 6065 |
| 10.9375 | 3185 | 9.19117647 | 2729 | 10.2022059 | 3136 |
| 5.46875 | 1465 | 4.59558824 | 1375 | 5.10110294 | 1539 |
| 2.734375 | 701 | 2.29779412 | 675 | 2.55055147 | 789 |
| 0 | 78 | 0 | 77 | 0 | 77 |

linear regressions were calculated in Prism [symbol]
indicates data missing or illegible when filed

Supplementary Tables 3 and 4 are found in the publication: Daniel L. Dunkelmann1, Sebastian B. Oehm, Adam T. Beattie, Jason W. Chin; “A 68-codon genetic code to incorporate four distinct non-canonical amino acids enabled by automated orthogonal mRNA discovery”, and are incorporated by reference in their entirety.

1. A method of designing a messenger RNA (mRNA) which is an orthogonal messenger RNA (O-mRNA) suitable for translation by an orthogonal ribosome (O-ribosome), wherein the mRNA comprises a 5′ untranslated region (5′ UTR) and an open reading frame (ORF), the method comprising:
(a) predicting the free energy difference between the free-folded state of the mRNA and the O-ribosome-bound initiation-competent state of the mRNA (ΔGₜₒₜ(O-ribo));
(b) introducing a modification into the 5′ UTR;
(c) predicting the new ΔGₜₒₜ(O-ribo) (ΔGₜₒₜ^{new}(O-ribo)) after modification;
(d) accepting the modification if said ΔGₜₒₜ^{new}(O-ribo) is more negative than the preceding ΔGₜₒₜ(O-ribo), and
accepting or rejecting the modification according to a probability distribution if said ΔGₜₒₜ^{new}(O-ribo) is more positive than the preceding ΔGₜₒₜ(O-ribo); and
(e) generating an O-mRNA sequence comprising the 5′ UTR which comprises the accepted modification(s).

2. The method of claim 1, wherein;
(i) ΔGₜₒₜ(O-ribo) is the sum of the free energy required to unfold the mRNA (ΔG_{unfolding}) and the free energy released upon the mRNA binding to the O-ribosome to form an O-ribosome-bound initiation-competent state (ΔG_{o-ribo binding});
(ii) the ΔGₜₒₜ^{new}(O-ribo) is more positive than the preceding ΔGₜₒₜ(O-ribo), the magnitude of the difference between said ΔGₜₒₜ^{new}(O-ribo) and said ΔGₜₒₜ(O-ribo) determines the probability of acceptance, wherein a smaller magnitude is associated with a higher chance of acceptance compared to a larger magnitude;
(iii) the probability distribution according to which the modification is accepted or rejected is:
$$exp ( \frac{\vert Δ {G}_{tot}^{new} ( O - ribo ) - Δ {G}_{tot} ( O - ribo ) \vert}{{T}_{SA}} )$$
wherein T_{SA} is the simulated annealing temperature;
(iv) the modification is or comprises a single nucleotide change, insertion, or deletion;
(v) steps (b) to (d) are iterated at least 200, 300, 400, 500, 1000, 5000, or 10000 times; or
steps (b) to (d) are iterated until at least 10, 50, 100, 250, or 500 consecutive iterations do not lead to a more negative ΔGₜₒₜ^{new}(O-ribo);
(vi) the 5′ UTR of step (a) is 35 nucleotides in length: or the modification is at any of 35 nucleotides of the 5′ UTR that are closest to the start codon;
(vii) the 5′ UTR of step (a) is according to a randomly generated sequence of nucleic acids;
(viii) the 5′ UTR of step (a) comprises a wild type Shine Dalgarno sequence;
(ix) the 2^{nd} ribosome is a wild type ribosome; or the 2^{nd} ribosome is an O-ribosome which differs from the first O-ribosome; and/or
(x) the method is implemented on a computer.

3. The method of claim 2, wherein:
(i) the O-ribosome comprises an orthogonal 16S rRNA and the mRNA comprises a Shine Dalgarno sequence, and the ΔGₜₒₜ(O-ribo) is predicted according to the following:
$$Δ {G}_{tot} ( O - ribo ) = ( Δ {G}_{mRNA - O - rRNA} + Δ {G}_{start} + Δ {G}_{spacing} - Δ {G}_{standby} ) + Δ {G}_{unfolding} ;$$
wherein
ΔG_{mRNA-O-rRNA} is the free energy of the predicted co-folded secondary structure of the last 9 nucleotides of the orthogonal 16S rRNA and the mRNA;
ΔGₛₜₐᵣₜ is the energy released from binding of an initiator tRNA to the start codon of the ORF;
ΔG_{spacing} is an energy penalty for non-optimal spacing length between the Shine Dalgarno sequence and the start codon;
ΔG_{standby} is the energy required to unfold secondary structures that sequester the four nucleotides upstream of the Shine Dalgarno sequence; and
ΔG_{unfolding} is the energy required to unfold secondary structures in the mRNA;
(ii) the T_{SA} is adjusted to maintain a 5-20% acceptance rate; and/or
(iii) the Shine Dalgarno sequence is five nucleotides from the start codon of the ORE.

4-6. (canceled)

7. The method of claim 1, wherein the method is for designing an mRNA which is an O-mRNA suitable for translation by an O-ribosome in a cell also comprising a second ribosome (2^{nd}-ribosome), wherein
step (a) comprises predicting the free energy difference between the free-folded state of the mRNA and the 2^{nd}-ribosome-bound initiation-competent state of the mRNA (ΔGₜₒₜ(2^{nd}-ribo));
step (c) comprises predicting the new ΔGₜₒₜ(2^{nd}-ribo) (ΔGₜₒₜ^{new}(2^{nd}-ribo) after modification;
step (d) comprises: accepting the modification if said ΔGₜₒₜ^{new}(O-ribo) is more negative than the preceding ΔGₜₒₜ(O-ribo) and said ΔGₜₒₜ^{new}(2^{nd}-ribo) is more positive than the preceding ΔGₜₒₜ(2^{nd}-ribo), and
accepting or rejecting the modification according to a probability distribution if said ΔGₜₒₜ^{new}(O-ribo) is more positive than the preceding ΔGₜₒₜ(O-ribo) or if said ΔGₜₒₜ^{new}(2^{nd}-ribo) is more negative than the preceding ΔGₜₒₜ(2^{nd}-ribo).

8. A method of designing an mRNA which is an O-mRNA suitable for translation by an O-ribosome in a cell also comprising a second ribosome (2^{nd}-ribosome), wherein the mRNA comprises a 5′ UTR and an ORF, wherein the method comprises:
(a) predicting the free energy difference between the free-folded state of the mRNA and the O-ribosome-bound initiation-competent state of the O-mRNA (ΔGₜₒₜ(O-ribo)) and predicting the free energy difference between the free-folded state of the mRNA and the 2^{nd}-ribosome-bound initiation-competent state of the mRNA (ΔGₜₒₜ(2^{nd}-ribo));
(b) introducing a modification into the 5′ UTR;
(c) predicting the new ΔGₜₒₜ(O-ribo) (ΔGₜₒₜ^{new}(O-ribo)) and the new ΔGₜₒₜ(2^{nd}-ribo) (ΔGₜₒₜ^{new}(2^{nd}-ribo) after modification;
(d) accepting the modification if said ΔGₜₒₜ^{new}(O-ribo) is more negative than the preceding ΔGₜₒₜ(O-ribo) and said ΔGₜₒₜ^{new}(2^{nd}-ribo) is more positive than the preceding ΔGₜₒₜ(2^{nd}-ribo), and
accepting or rejecting the modification according to a probability distribution if said ΔGₜₒₜ^{new}(O-ribo) is more positive than the preceding ΔGₜₒₜ(O-ribo) or if said ΔGₜₒₜ^{new}(2^{nd}-ribo) is more negative than the preceding ΔGₜₒₜ(2^{nd}-ribo); and
(e) generating an O-mRNA sequence comprising the 5′ UTR which comprises the accepted modification(s).

9. The method of claim 7, wherein:
(i) ΔGₜₒₜ(2^{nd}-ribo) is the sum of the free energy required to unfold the mRNA (ΔG_{unfolding}) and the free energy released upon the mRNA binding to the 2^{nd}-ribosome to form a 2^{nd}-ribosome-bound initiation-competent state (ΔG_{2nd ribo binding});
(ii) the ΔGₜₒₜ^{new}(O-ribo) is more positive than the preceding ΔGₜₒₜ(O-ribo) or the ΔGₜₒₜ^{new}(2^{nd}-ribo) is more negative than the preceding ΔGₜₒₜ(2^{nd}-ribo), the magnitude of the difference between said ΔGₜₒₜ^{new}(O-ribo) and said ΔGₜₒₜ(O-ribo) or between said ΔGₜₒₜ^{new}(2^{nd}-ribo) and said ΔGₜₒₜ(2^{nd}-ribo) determines the probability of acceptance, wherein a smaller magnitude is associated with a higher chance of acceptance compared to a larger magnitude; and/or
(iii) steps (b) to (d) are iterated until at least 10, 50, 100, 250, or 500 consecutive iterations do not lead to a more negative ΔGₜₒₜ^{new}(O-ribo) or a more positive ΔGₜₒₜ^{new}(2^{nd}-ribo).

10. The method of claim 9, wherein the 2^{nd}-ribosome comprises a 16S rRNA and the mRNA comprises a Shine Dalgarno sequence, and the ΔGₜₒₜ(2^{nd}-ribo) is predicted according to the following:
$$Δ {G}_{tot} ( {2}^{nd} - ribo ) = ( Δ {G}_{mRNA - 2 nd - rRNA} + Δ {G}_{start} + Δ {G}_{spacing} - Δ {G}_{standby} ) + Δ {G}_{unfolding} ;$$
wherein
ΔG_{mRNA-2nd-rRNA} is the free energy of the predicted co-folded secondary structure of the last 9 nucleotides of the 16S rRNA and the mRNA;
ΔGₛₜₐᵣₜ is the energy released from binding of an initiator tRNA to the start codon of the ORF;
ΔG_{spacing} is an energy penalty for non-optimal spacing length between the Shine Dalgarno sequence and the start codon;
ΔG_{standby} is the energy required to unfold secondary structures that sequester the four nucleotides upstream of the Shine Dalgarno sequence; and
ΔG_{unfolding} is the energy required to unfold secondary structures in the mRNA.

11. (canceled)

12. The method of claim 7, wherein
step (a) comprises calculating ΔGₜₒₜ(opt) according to the formula:
$$Δ {G}_{tot} ( opt ) = Δ {G}_{tot} ( O - ribo ) - X * Δ {G}_{tot} ( {2}^{nd} - ribo ) ;$$
step (c) comprises calculating ΔGₜₒₜ^{new}(opt) according to the formula:
$$Δ {G}_{tot}^{new} ( opt ) = Δ {G}_{tot}^{new} ( O - ribo ) - X * Δ {G}_{tot}^{new} ( {2}^{nd} - ribo ) ;$$
and
step (d) comprises: accepting the modification if said ΔGₜₒₜ^{new}(opt) is more negative than the preceding ΔGₜₒₜ(opt), and
accepting or rejecting the modification according to a probability distribution if said ΔGₜₒₜ^{new}(opt) is more positive than the preceding ΔGₜₒₜ(opt);
wherein X is from 0.1 to 2, or X is 0.5.

13. The method of claim 12, wherein;
(i) when the ΔGₜₒₜ^{new}(opt) is more positive than the preceding ΔGₜₒₜ(opt), the magnitude of the difference between said ΔGₜₒₜ^{new}(opt) and said ΔGₜₒₜ(opt) determines the probability of acceptance, wherein a smaller magnitude is associated with a higher chance of acceptance compared to a larger magnitude;
(ii) the probability distribution according to which the modification is accepted or rejected is:
$$exp ( \frac{Δ {G}_{tot}^{new} ( opt ) - Δ {G}_{tot} ( opt )}{{T}_{SA}} )$$
wherein T_{SA} is the simulated annealing temperature; and/or
(iii) steps (b) to (d) are iterated until at least 10, 50, 100, 250, or 500 consecutive iterations do not lead to a more negative ΔGₜₒₜ^{new}(opt).

14. (canceled)
$$exp ( \frac{Δ {G}_{tot}^{new} ( opt ) - Δ {G}_{tot} ( opt )}{{T}_{SA}} )$$

15. The method of claim 13, wherein the T_{SA} is adjusted to maintain a 5-20% acceptance rate.

16. (canceled)

17. The method of claim 1, wherein
step (b) comprises introducing a modification into the 5′ UTR, or the exchange of any one of codons 2 to 20, 2 to 15, 2 to 12, 2 to 10, or 2 to 5 within the ORF with a synonymous codon; and
step (e) comprises generating an O-mRNA sequence comprising the 5′ UTR and the ORF which comprise the accepted modification(s).

18. The method of claim 17, wherein step (b) comprises introducing a modification comprising a single nucleotide change, insertion, or deletion into the 5′ UTR, or the exchange of any one of codons 2 to 12 within the ORF with a synonymous codon.

19-25. (canceled)

26. The method of claim 1, wherein the O-ribosome comprises an orthogonal anti-Shine Dalgarno sequence and the 5′ UTR of step (a) comprises an orthogonal Shine Dalgarno sequence (O-SD) that is predicted to be perfectly complementary to the orthogonal anti-Shine Dalgarno sequence.

27. The method of claim 26, wherein step (b) does not comprise introducing a modification into the five-nucleotide core of the O-SD.

28-31. (canceled)

32. A method for producing a nucleic acid sequence encoding an exogenous protein for translation by an O-ribosome, wherein the sequence of an O-mRNA is designed according to the method of claim 1, and then a nucleic acid molecule is produced encoding said sequence.

33. A system for designing an orthogonal messenger RNA (O-mRNA) for translation by an orthogonal ribosome (O-ribosome), the system comprising:
a processor; and
one or more computer-readable storage media having stored thereon instructions for execution on said processor to perform the method of claim 1.

34. A computer program product comprising a non-transitory machine readable medium storing program code that, when executed by one or more processors of a computer system, causes the computer system to implement the method of claim 1.

35. A method of designing an operon encoding at least two exogenous tRNAs for expression in a host cell comprising an endogenous genome encoding endogenous tRNAs, the method comprising:
(i) generating permutations of arrangements of the at least two exogenous tRNAs;
(ii) identifying, within the endogenous genome, adjacent pairs of endogenous tRNAs with the highest level of sequence identity to each adjacent pair of exogenous tRNAs within each permutation of the at least two exogenous tRNAs;
(iii) identifying the intergenic region in the endogenous genome between each of the identified adjacent pairs of endogenous tRNAs;
(iv) generating a plurality of sequences encoding each permutation of the at least two exogenous tRNAs and comprising the identified intergenic region(s) positioned between each associated adjacent pair of the exogenous tRNAs; and
(v) selecting a sequence from said plurality of sequences for inclusion in the operon encoding the at least two exogenous tRNAs.

36. The method of claim 35, wherein:
(a) the selection of step (v) is made from ranked list of the plurality of sequences, wherein the ranked list is created by ranking each of the plurality of sequences based on the sum of the sequence identity between the at least two exogenous tRNAs and the corresponding endogenous tRNAs used to define the intergenic regions,
(b) the sequence identity of step (ii) is calculated by comparing the acceptor stem sequences of the endogenous tRNAs to the acceptor stem sequences of the exogenous tRNAs;
(c) the minimum intergenic region to be considered is 5, 10, 15, 20, or 25 base pairs and the maximum is 50, 75, 100, 125, or 150 base pairs;
(d) the method is for designing an operon encoding at least three, at least four, at least five, or at least six exogenous tRNAs;
(e) the method is implemented on a computer.

37. (canceled)

38. The method of claim 36, wherein:
(a) the first seven and last eight nucleotides, not including the CCA end, of the tRNAs are compared; and/or
(b) the minimum intergenic region to be considered is 10 base pairs and the maximum is 100 base pairs.

39-42. (canceled)

43. A method for producing a nucleic acid sequence encoding an operon comprising at least two exogenous tRNAs, wherein the sequence of the nucleic acid is designed according to the method of claim 35, and then a nucleic acid is produced encoding said sequence.

44. A system for designing an operon comprising at least two exogenous tRNAs, the system comprising:
a processor; and
one or more computer-readable storage media having stored thereon instructions for execution on said processor to perform the method of claim 35.

45. A computer program product comprising a non-transitory machine readable medium storing program code that, when executed by one or more processors of a computer system, causes the computer system to implement the method of claim 35.

46. A nucleic acid, wherein the nucleic acid comprises an operon that is obtained or is obtainable by the method of claim 43.

47. A host cell comprising an endogenous genome, wherein the host cell comprises a nucleic acid encoding an operon comprising at least two exogenous tRNAs, and wherein the nucleic acid sequence between each pair of exogenous tRNAs is an intergenic sequence derived from the endogenous genome.

48. The host cell of claim 47, wherein the operon is obtained or is obtainable by the method of claim 43.

49. The method of claim 35, wherein the host cell is a prokaryotic cell.

50. The method of claim 49, wherein the prokaryotic cell is a bacterial cell.

51. The method of claim 50, wherein the bacterial cell is E. coli and the endogenous genome is an E. coli genome.

52. A method of designing an operon comprising at least two exogenous ORFs for expression in a host cell, wherein the method comprises:
(i) generating a plurality of 5′ UTR sequences for each of the at least two exogenous ORFs, wherein each 5′ UTR sequence is optimised for a negative predicted free energy difference between the free-folded state of an mRNA comprising said 5′ UTR sequence and the exogenous ORF and the ribosome-bound initiation-competent state of said mRNA (ΔGₜₒₜ(ribo));
(ii) predicting the ΔGₜₒₜ(ribo) for each of the 5′ UTR sequences when positioned 5′ to the exogenous ORF for which said 5′ UTR was optimised and positioned 3′ to each one of the remaining at least two exogenous ORFs; and
(iii) selecting an arrangement of the 5′ UTR sequences and the at least two exogenous ORFs.

53. The method of claim 52, wherein:
(a) step (iii) comprises selecting an arrangement of the 5′ UTR sequences and the at least two exogenous ORFs wherein:
the sum of the ΔGₜₒₜ(ribo) for all 5′ UTR/exogenous ORF pairs is the most negative; and/or
the mean of the ΔGₜₒₜ(ribo) for all 5′ UTR/exogenous ORF pairs is the most negative; and/or
each 5′ UTR/exogenous ORF pair has a ΔGₜₒₜ(ribo) which is more negative than a target ΔGₜₒₜ(ribo),
(b) step (i) comprises generating two, three, four, five, or more 5′ UTR sequences for each of the at least two exogenous ORFs;
(c) at least one or all of the at least two exogenous ORFs is an aminoacyl-tRNA synthetase;
(d) the method is for designing an operon encoding at least three, at least four, at least five, or at least six exogenous ORFs;
(e) ΔGₜₒₜ(ribo) is the sum of the free energy required to unfold the mRNA (ΔG_{unfolding}) and the free energy released upon the mRNA binding to a ribosome to form a ribosome-bound initiation-competent state (ΔG_{ribo binding}); and/or
(f) the method is implemented on a computer.

54-57. (canceled)

58. The method of claim 52, wherein ΔGₜₒₜ(ribo) is the sum of the free energy required to unfold the mRNA (ΔG_{unfolding}) and the free energy released upon the mRNA binding to a ribosome to form a ribosome-bound initiation-competent state (ΔG_{ribo binding}), wherein the 5′ UTR comprises a Shine Dalgarno sequence, and the ΔGₜₒₜ(ribo) is predicted according to the following:
$$Δ {G}_{tot} ( ribo ) = ( Δ {G}_{mRNA - rRNA} + Δ {G}_{start} + Δ {G}_{spacing} - Δ {G}_{standby} ) + Δ {G}_{unfolding} ;$$
wherein
ΔG_{mRNA-rRNA} is the free energy of a predicted co-folded secondary structure of the last 9 nucleotides of a 16S rRNA and the mRNA;
ΔGₛₜₐᵣₜ is the energy released from binding of an initiator tRNA to the start codon of the sequence encoding the exogenous ORF;
ΔG_{spacing} is an energy penalty for non-optimal spacing length between the Shine Dalgarno sequence and the start codon of the sequence encoding the exogenous ORF;
ΔG_{standby} is the energy required to unfold secondary structures that sequester the four nucleotides upstream of the Shine Dalgarno sequence; and
ΔG_{unfolding} is the energy required to unfold secondary structures in the mRNA.

59. The method of claim 52, wherein step (i) comprises:
(a) introducing a modification into the 5′ UTR;
(b) predicting the new ΔGₜₒₜ(ribo) (ΔGₜₒₜ^{new}(ribo)) after modification;
(c) accepting the modification if said ΔGₜₒₜ^{new}(ribo) is more negative than the preceding ΔGₜₒₜ(ribo), and
accepting or rejecting the modification according to a probability distribution if said ΔGₜₒₜ^{new}(ribo) is more positive than the preceding ΔGₜₒₜ(ribo); and
(d) generating a 5′ UTR sequence comprising the accepted modification(s).

60. The method of claim 59, wherein;
(A) when the ΔGₜₒₜ^{new}(ribo) is more positive than the preceding ΔGₜₒₜ(ribo), the magnitude of the difference between said ΔGₜₒₜ^{new}(ribo) and said ΔGₜₒₜ(ribo) determines the probability of acceptance, wherein a smaller magnitude is associated with a higher chance of acceptance compared to a larger magnitude;
(B) the probability distribution according to which the modification is accepted or rejected is:
$$exp ( \frac{\vert Δ {G}_{tot}^{new} ( ribo ) - Δ {G}_{tot} ( ribo ) \vert}{{T}_{SA}} )$$
wherein T_{SA} is the simulated annealing temperature:
(C) the modification is or comprises a single nucleotide change, insertion, or deletion;
(D) step (a) comprises introducing a modification into the 5′ UTR, or the exchange of any one of codons 2 to 20, 2 to 15, 2 to 12, 2 to 10, or 2 to 5 with a synonymous codon within the sequence encoding the exogenous ORF; and
step (d) comprises generating a sequence comprising the 5′ UTR and the ORF which comprise the accepted modification(s); and/or
(E) steps (a) to (c) are iterated at least 200, 300, 400, 500, 1000, 5000, or 10000 times: or steps (a) to (c) are iterated until at least 10, 50, 100, 250, or 500 consecutive iterations do not lead to a more negative ΔGₜₒₜ^{new}(ribo).

61. (canceled)

62. The method of claim 60, wherein:
(1) the T_{SA} is adjusted to maintain a 5-20% acceptance rate; and/or
(2) step (a) comprises introducing a modification comprising a single nucleotide change, insertion, or deletion into the 5′ UTR, or the exchange of any one of codons 2 to 12 within the ORF with a synonymous codon.

63-68. (canceled)

69. A method for producing a nucleic acid sequence encoding a polycistronic operon comprising at least two exogenous ORFs, wherein the sequence of the nucleic acid is designed according to the method of claim 52, and then a nucleic acid is produced according to said sequence.

70. A system for designing a polycistronic operon comprising at least two exogenous ORFs, the system comprising:
a processor; and
one or more computer-readable storage media having stored thereon instructions for execution on said processor to perform the method of claim 52.

71. A computer program product comprising a non-transitory machine readable medium storing program code that, when executed by one or more processors of a computer system, causes the computer system to implement the method of claim 52.

72. A nucleic acid, wherein nucleic acid comprises an operon that is obtained or is obtainable by the method of claim 69.

73. A host cell comprising a nucleic acid encoding an operon that is obtained or is obtainable by the method of claim 69.

74. The method of claim 52, wherein the host cell is a prokaryotic cell.

75. The method of claim 74, wherein the prokaryotic cell is a bacterial cell.

76. The method of claim 75, wherein the bacterial cell is E. coli and the endogenous genome is an E. coli genome.

77. A host cell comprising:
a nucleic acid sequence encoding an O-mRNA which encodes an exogenous protein, wherein the O-mRNA is obtained or is obtainable by the method of claim 32, and wherein the O-mRNA comprises at least two types of orthogonal codon;
a nucleic acid sequence comprising an O-tRNA operon encoding at least two orthogonal tRNAs, wherein the at least two orthogonal tRNAs are capable of decoding said at least two types of orthogonal codon, wherein the operon is obtained or is obtainable by a method for producing a nucleic acid sequence encoding an operon comprising at least two exogenous tRNAs, wherein the sequence of the nucleic acid is designed according to a method of designing an operon encoding at least two exogenous tRNAs for expression in a host cell comprising an endogenous genome encoding endogenous tRNAs, wherein the method of designing an operon comprises:
(i) generating permutations of arrangements of the at least two exogenous tRNAs;
(ii) identifying, within the endogenous genome, adjacent pairs of endogenous tRNAs with the highest level of sequence identity to each adjacent pair of exogenous tRNAs within each permutation of the at least two exogenous tRNAs;
(iii) identifying the intergenic region in the endogenous genome between each of the identified adjacent pairs of endogenous tRNAs;
(iv) generating a plurality of sequences encoding each permutation of the at least two exogenous tRNAs and comprising the identified intergenic region(s) positioned between each associated adjacent pair of the exogenous tRNAs; and
(v) selecting a sequence from said plurality of sequences for inclusion in the operon encoding the at least two exogenous tRNAs,
and then a nucleic acid is produced encoding said sequence;
a nucleic acid sequence comprising an orthogonal aminoacyl-tRNA synthetase (O-aaRS) operon encoding at least two O-aaRSs, wherein the at least two O-aaRSs form O-aaRS-O-tRNA pairs with the at least two orthogonal tRNAs, wherein the operon is obtained or is obtainable by a method for producing a nucleic acid sequence encoding a polycistronic operon comprising at least two exogenous ORFs, wherein the sequence of the nucleic acid is designed according to a method of designing an operon comprising at least two exogenous ORFs for expression in a host cell, wherein the method of designing an operon comprises:
(i) generating a plurality of 5′ UTR sequences for each of the at least two exogenous ORFs, wherein each 5′ UTR sequence is optimised for a negative predicted free energy difference between the free-folded state of an mRNA comprising said 5′ UTR sequence and the exogenous ORF and the ribosome-bound initiation-competent state of said mRNA (ΔGₜₒₜ(ribo));
(ii) predicting the ΔGₜₒₜ(ribo) for each of the 5′ UTR sequences when positioned 5′ to the exogenous ORF for which said 5′ UTR was optimised and positioned 3′ to each one of the remaining at least two exogenous ORFs; and
(iii) selecting an arrangement of the 5′ UTR sequences and the at least two exogenous ORFs,
and then a nucleic acid is produced according to said sequence; and
an orthogonal ribosome.

78. The host cell of claim 77, wherein:
(a) the O-mRNA comprises at least three or four types of orthogonal codon;
the O-tRNA operon encodes at least three or four orthogonal tRNAs which are capable of decoding said at least three or four orthogonal codons;
the O-aaRS operon encodes at least three or four O-aaRSs which form O-aaRS-O-tRNA pairs with the at least three or four orthogonal tRNAs; and/or
(b) the host cell is a prokaryotic cell.

79-80. (canceled)

81. The host cell of claim 78, wherein the prokaryotic cell is a bacterial cell.

82. The host cell of claim 81, wherein the bacterial cell is E. coli.

83. A method of producing a polypeptide, comprising:
providing a host cell of claim 78;
incubating the host cell in the presence of a first non-canonical amino acid, wherein the first non-canonical amino acid is a substrate for the one of the O-aaRSs; and
incubating the host cell to allow incorporation of the first non-canonical amino acid into the polypeptide via the O-aaRS-O-tRNA pair.

84. The method of claim 83, comprising:
incubating the host cell in the presence of a second non-canonical amino acid, wherein the second non-canonical amino acid is a substrate for the one of the O-aaRSs; and
incubating the host cell to allow incorporation of the second non-canonical amino acid into the polypeptide via the O-aaRS-O-tRNA pair.

85. The method of claim 84, comprising:
(A) incubating the host cell in the presence of a third non-canonical amino acid, wherein the third non-canonical amino acid is a substrate for the one of the O-aaRSs; and
incubating the host cell to allow incorporation of the third non-canonical amino acid into the polypeptide via the O-aaRS-O-tRNA pair; and/or
(B) incubating the host cell in the presence of a fourth non-canonical amino acid, wherein the fourth non-canonical amino acid is a substrate for the one of the O-aaRSs; and
incubating the host cell to allow incorporation of the fourth non-canonical amino acid into the polypeptide via the O-aaRS-O-tRNA pair.

86. (canceled)

87. A polypeptide obtained or obtainable by the method of claim 85.
